(19) United States
(12) Patent Application Publication
(10) Pub. No.: US 2019/0203227 A1
(43) Pub. Date: Jul. 4, 2019

(54) LIGHT-CONTROLLED GENE DELIVERY WITH VIRUS VECTORS THROUGH INCORPORATION OF OPTOGENETIC PROTEINS AND GENETIC INSERTION OF NON-CONFORMATIONALLY CONSTRAINED PEPTIDES

(71) Applicant: WILLIAM MARSH RICE UNIVERSITY, Houston, TX (US)

(72) Inventors: Michelle Ho, Los Altos, CA (US); Junghae Suh, Houston, TX (US); Eric Gomez, Houston, TX (US); Justin Judd, Berkeley, CA (US); Jeffrey Tabor, Houston, TX (US); Karl Gerhardt, Houston, TX (US)

(21) Appl. No.: 15/927,798

(22) Filed: Mar. 21, 2018

Related U.S. Application Data
(63) Continuation of application No. PCT/US2016/053200, filed on Sep. 22, 2016.
(60) Provisional application No. 62/222,047, filed on Sep. 22, 2015.
(60) Provisional application No. 62/221,754, filed on Sep. 22, 2015.

(51) Int. Cl. C12N 15/86; C12N 15/52; C07K 14/415; C07K 14/195

(52) U.S. Cl. CPC C12N 15/86; C12N 15/52; C12N 2310/20; C07K 14/195; C12N 2750/14141; C07K 14/415

(57) ABSTRACT

This invention describes light-controlled delivery to the nucleus of target cells via viral vectors modified using optogenetic tools. This invention also describes tools for the display of proteins on the surface of adeno-associated virus using enzymatic tools to display the proteins in a more favorable thermodynamic configuration to enhance activity of the proteins or their targets.

### CROSS-REFERENCE TO RELATED APPLICATIONS

The present application is a continuation application of International Application No. PCT/US 16/53200, filed Sep. 22, 2016, which claims benefit of U.S. Provisional Application No. 62/222,047, filed on Sep. 22, 2015 and to U.S. Provisional Application No. 62/221,754, filed on Sep. 22, 2015, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Sep. 22, 2016, is named 15-21018-WO_SL.txt and is 520,224 bytes in size.

### BACKGROUND

Viruses are genetically encoded nanoparticles with regular geometry, monodispersity, and self-assembly. These properties, coupled with an innate ability to infect and deliver nucleic acid cargo into host cells, have fueled efforts toward developing more potent and controllable viral nanoparticles (VNPs) for precision gene delivery application ranging from fundamental biological studies to clinical translation. However, controlling the specificity and efficiency of delivery remain as considerable challenges limiting the full potential of virus-enabled approaches. Many avenues have been pursued to improve the functionality of viruses, yielding a diverse suite of “bionic” viruses that are part natural and part synthetic; yet more advances are required to transform naturally occurring viruses into well-controlled and predictable nanodevices.

Adeno-associated virus (AAV) vectors can deliver genetic material to target cells including, but not limited to genes, RNA interference (RNAi), or CRISPR/Cas genome editing tools. A significant rate-limiting step and major determinant of effective gene delivery using AAV is inefficient nuclear entry; although AAV is considered an efficient gene delivery vector, most virions added to host cells appear to remain outside the nucleus. Additionally, off-target gene delivery by AAV poses a significant risk of undesired side effects in in vivo applications. The present disclosure provides a solution that addresses both of these problems.

A promising approach for engineering programmable nanodevices, such as AAV, is to encode stimulus-responsive properties. A number of synthetic nanoparticles have been designed such that detection of a particular stimulus leads to a physiochemical change in the nanoparticle, resulting in cargo delivery. For example, chemical ligands, pH, enzymatic reactions, redox reactions, temperature, and magnetic fields have served as input stimuli for various non-viral nanocarriers. Despite these promising advances, non-viral delivery systems still display lower delivery efficiencies compared to viral vectors. For this reason, stimulus-responsive virus-based platforms that respond to pH, chemicals and extracellular proteases have been developed.

Although the use of tissue-specific stimuli may be beneficial for certain applications, externally applied stimuli can provide a more quantitatively controllable delivery process in both space and time. Light represents an attractive stimulus over chemical or biological stimuli because its intensity, duration, spatial pattern, and wavelength can all be precisely modulated in real time with the proper equipment and light configuration. In in vitro tissue models, light has been used with a resolution of microns to pattern proteins that direct cell processes like migration and differentiation. Light can also non-invasively penetrate the skin and is generally considered safe for use in mammalian tissues.

Optogenetics offers a molecular toolbox of light-switchable proteins. Among the photo-switchable proteins, phytochrome-family proteins are powerful because they can be activated by one wavelength and deactivated by a second wavelength, allowing control over the degree of activation in live cells in space and time. For example, Phytochrome B (PhyB) has been used for light-switchable transcription, signal cascade activation, actin nucleation, autocatalytic protein splicing, and pseudopodia elongation. The apo form of PhyB from A. thaliana covalently binds to the tetrapyrrole chromophore phycocyanobilin (PCB) to form the holoprotein, after which PhyB rapidly associates with and dissociates from phytochrome interacting factor 6 (PIF6) upon absorption of red (R, λₘₐₓ=650 nm) photons or far-red (FR, λₘₐₓ=750 nm) photons, respectively. The PhyB/PIF6 system dimerizes in seconds, is amenable to fusion proteins, and is non-toxic to mammalian cells.

U.S. Patent Application Publication No. 2013/0330766 A1 describes another suite of tools for manipulation of the viral capsid to enhance and/or control gene delivery using viral vectors. U.S. 2013/0330766 A1 discloses “peptide locks” where enzymatically cleavable motifs are inserted flanking a peptide or protein that has been inserted into the capsid protein of an adeno-associated virus. These protease-susceptible motifs allow for release of a “peptide lock” upon exposure to the a protease or combination of proteases which can cleave the enzymatically cleavable motifs.

This disclosure describes compositions, methods of making said compositions and methods for using said compositions which incorporate the advantages of viral delivery systems with the spatial and temporal control offered by optogenetic tools to offer improved gene delivery systems. In the context of AAV-mediated gene delivery, these tools can provide improved nuclear delivery of genetic material and more specifically targeted delivery of genetic material to target cells.

The present disclosure also provides compositions, methods of making said composition and methods for using said compositions which incorporate the advantages of viral delivery systems with enzymatic cleavage sites incorporate in the viral capsid to enable surface display of peptides and proteins in a more favorable thermodynamic conformation, such as a linear conformation. In the context of viral-mediated gene delivery, these tools can provide for improved display of peptides and proteins inserted into the viral capsid which may facilitate improved interaction with a target and/or target cell.

### SUMMARY

The present disclosure is directed to light-controllable, viral-based gene delivery vectors incorporating optogenetic proteins or optogenetic binding partners and methods of use of such vectors. These vectors and methods can provide improved, tunable nuclear delivery of genetic material, endosomal escape as well as improved cell binding and both spatial and temporal control of gene delivery in a cell population. The present disclosure also provides nucleic acids and amino acids useful in making and using such vectors as well as kits for the use of vectors herein.

The present disclosure is also directed to viral-based gene delivery vectors incorporating an enzymatic cleavage motif for linearizing or conformationally unconstraining a peptide or protein inserted into a varial capsid to improve the efficiency of methods using the peptide or protein for binding and/or to improve cell binding, endosomal escape and nuclear localization.

In an embodiment, a virus is provided which includes a capsid protein and an optogenetic binding partner, wherein at least a portion of the optogenetic binding partner is displayed on the surface of the virus, and wherein the optogenetic binding partner is linked to the capsid protein by a direct amino acid linkage or a linker.

In some embodiments, the virus which includes a capsid protein and an optogenetic binding partner further includes an enzymatic cleavage motif adjacent to the optogenetic binding partner, wherein the enzymatic cleavage motif does not inactivate other biologically active motifs on the surface of the virus.

In another embodiment, a virus is provided which includes a capsid protein and an optogenetic protein, wherein at least a portion of the optogenetic protein is displayed on the surface of the virus and wherein the optogenetic protein is linked to the capsid protein by a direct amino acid linkage or a linker.

In some embodiments, the virus which includes a capsid protein and an optogenetic protein further includes an enzymatic cleavage motif adjacent to the optogenetic protein, wherein the enzymatic cleavage motif does not inactivate other biologically active motifs on the surface of the virus.

In another embodiment, a method for delivering a nucleic acid molecule to the nucleus of a target cell includes the steps of obtaining a virus with at least a portion of an optogenetic binding partner displayed on its surface, delivering the virus to a target cell containing an optogenetic protein capable of binding to the optogenetic binding partner and having a nuclear localization signal, and exposing the target cell to light of a sufficient wavelength to induce a conformational change in the optogenetic protein to allow binding of the optogenetic protein and the optogenetic binding partner, enhancing delivery of the virus.

In another embodiment, a method for delivering a nucleic acid molecule to the nucleus of a target cell includes the steps of obtaining a virus with at least a portion of an optogenetic protein displayed on its surface, delivering the virus to a target cell containing an optogenetic binding partner capable of binding to the optogenetic protein and having a nuclear localization signal, and exposing the target cell to light of a sufficient wavelength to induce a conformational change in the optogenetic protein to allow binding of the optogenetic protein and the optogenetic binding partner, enhancing delivery of the virus.

In still another embodiment, a method for delivering a nucleic acid molecule to the nucleus of a target cell includes the steps of obtaining a virus with at least a portion of an optogenetic protein having a nuclear localization signal displayed on its surface which is either exposed or occluded based on the conformation of the optogenetic protein, delivering the virus to a target cell, and exposing the target cell to light of a sufficient wavelength to induce a conformational change in the optogenetic protein to allow exposure of the nuclear localization signal, enhancing delivery of the virus.

In another embodiment, a method comprises providing a virus having one or more peptides genetically encoded into the capsid so as to be at least partially exposed to the surface of the capsid and an enzymatic cleavage motif cleavable by an enzyme genetically encoded into the capsid adjacent to the one or more peptides, and treating the virus with the enzyme to cleave the enzymatic cleavage motif, allowing at least a portion of the one or more peptides to be tethered to the capsid surface at either the C-terminal or N-terminal end.

The present disclosure also provides for nucleic acids encoding and amino acids comprising at least a portion of the viruses having an optogenetic binding partner, optogenetic protein and/or enzymatic cleavage motif.

This summary is provided to introduce disclosure, certain aspects, advantages and novel features of the invention in a simplified form that are further described below in the detailed description. This summary is not intended to identify key features or essential features of the claimed subject matter, nor is it intended to be used to limit the scope of the claimed subject matter.

### BRIEF DESCRIPTION OF THE DRAWINGS

The patent or application file contains at least one drawing executed in color. Copies of this patent or patent application publication with color drawings will be provided by the office upon request and payment of the necessary fee.

The summary above, as well as the following detailed description of illustrative embodiments, is better understood when read in conjunction with the appended drawings. For the purpose of illustrating the present disclosure, exemplary constructions of the disclosure are shown in the drawings. However, the disclosure is not limited to specific methods and instrumentalities disclosed herein.

All error bars shown in the figures are standard error of the mean (SEM) unless otherwise noted.

FIG. 1 depicts the adeno-associated virus (AAV) particle and a graphical representation of the 4.7 kB genome of AAV with the rep and cap genes and showing the alignment of the sequences of VP1, VP2 and VP3 from the cap open-reading frame (ORF).

FIG. 2A depicts certain embodiments of the invention where the nuclear uptake and expression in cells is tuned by altering the intensity of light (“tunable intensity”), controlling the timing of exposure to light (“temporal dynamics”) and controlling the area which is exposed to light (“patterning”).

FIG. 2B depicts the formation of the holoprotein of PhyB with its chromophore PCB and the association (binding) of PIF6 to the PhyB holoprotein under red light (650 nm) conditions and dissociation under far red light (750 nm) conditions.

FIG. 2C depicts a flow diagram showing the alternative splicing of the cap gene of AAV and leaky scanning to yield VP1, VP2 and VP3, translation of the corresponding capsid subunits which can be combined with a desired transgene of interest and allowed to self-assemble into the capsid with the transgene encapsulated.

FIG. 3A depicts a peptide lock embodiment where peptide “locks” are located on the viral surface and include two enzymatically cleavable motifs that are cleavable by an enzyme for unlocking the virus. Figure discloses SEQ ID NO: 29.

FIG. 3B depicts the expected activity, based on reported specificity constants for each matrix metalloprotease (MMP) against the indicated peptide substrate, and the observed activity, as % GFP⁺ cells for AAV with a peptide lock incorporating the cleavage motifs IPVSLRSG (SEQ ID NO: 1) or IPESLRAG (SEQ ID NO: 2).

FIG. 3C depicts an alternative peptide lock embodiment where the peptide “locks” located on the surface contain two cleavage sequences, one recognized by a protease and one recognized by a different protease, e.g. a MMP. Figure discloses SEQ ID NO: 30.

FIG. 3D depicts the alternative embodiment of FIG. 3C where, upon pre-treatment with protease, the peptide “lock” presents as a linearized peptide, allowing the different protease, e.g. a MMP, improved access to the second cleavage site, enabling the expected activity of the protease for the substrate.

FIG. 3E depicts the alternative embodiment of FIG. 3C, where each cleavage leaves at least some of the inserted amino acids on the surface of the virus.

FIG. 4A depicts the activity, as % GFP⁺ cells, for several variants constructed using the alternative embodiment of FIG. 3C and tested with or without pre-treatment with the protease and with or without treatment with the different protease, e.g. MMP-2 and MMP-7.

FIG. 4B depicts a silver stained gel for the ePAV4 variant from FIG. 4A treated with or without protease and with or without MMP-2, MMP-7 or MMP-9.

FIG. 5A depicts a graphical alignment of the capsid proteins of AAV2 as expressed within a construct expressing native VP1, VP2 and VP3 (wt); a construct expressing VP2 independently with an optogenetic binding partner, phytochrome interacting factor 6 (PIF6) inserted at the N-terminus of VP2 with a separate construct expressing VP1 and VP3 (VNP-2-PIF6), and a construct expressing VP1 and VP2 with PIF6 inserted at the N-terminus of VP2 and at M138 of VP1 with a separate construct expressing VP3 (VNP-1,2-PIF6). FIG. 5A also depicts a visual representation of the viral phenotypes produced from the wild-type construct and both VNP-2-PIF6 and VNP-1.2-PIF6. The “genotype” scale bar=300 base pairs, while the “phenotype” scale bar=10 nm (PIF6 not drawn to scale).

FIG. 5B depicts western blots of wild-type, VNP-2-PIF6 and VNP-1,2-PIF6 AAV2 viruses using a monoclonal anti-VP1, 2, 3 antibody after expression in HEK293T cells.

FIG. 5C depicts electron micrographs of wild-type, VNP-2-PIF6 and VNP-1,2-PIF6 viruses after expression in HEK293T cells. Black scale bar=100 nm, white scale bar=15 nm.

FIG. 5D depicts the results of a heparin binding assay using wild-type AAV2 and VNP-2-PIF6. The y-axis represents the fraction of total viral genomes quantified by qPCR. Error bars are SEM from 2 independent experiments conducted in duplicate.

FIG. 5E depicts the transduction index (TI) for wtAAV2, VNP-2-PIF6 and VNP-1,2,-PIF6 in HEK293T cells at multiplicity of infection (MOI) of 1,000, 5,000 and 10,000. “**” indicates a p-value <0.05.

FIG. 5F depicts the percentage of cells positive for GFP expression after exposure to wtAAV2, VNP-2-PIF6 or VNP-1,2-PIF6 at MOI of 1,000, 5,000 and 10,000.

FIG. 5G depicts the mean fluorescence intensity for cells after exposure to wtAAV2, VNP-2-PIF6 or VNP-1,2-PIF6 at MOI of 1,000, 5,000 and 10,000.

FIG. 6A depicts a Western blot of fractions of PhyB651-His₆ from nickel purification after expression in E. coli. F=flow through; W1=first wash; W2=second wash; W3=third wash, E1=first elution; E2=second elution.

FIG. 6B depicts a Western blot of fractions of PhyB917-His6 from nickel purification after expression in Dictyostelium discoideum. F=flow through; W1=first wash; W2=second wash; W3=third wash, E1=first elution; E2=second elution.

FIG. 6C depicts coomassie-stained gels corresponding to the fractions of PhyB651-His₆ in FIG. 6A.

FIG. 6D depicts Coomassie-stained gels corresponding to the fractions of PhyB917-His6 in FIG. 6B.

FIG. 7A depicts an in vitro binding assay strategy for assessing viral binding to PhyB proteins. VNP-PIF6 is equivalent to VNP-2-PIF6. Figure discloses “His6” as SEQ ID NO: 23.

FIG. 7B depicts the capture efficiency under far-red (FR) light conditions and red (R) light conditions for wtAAV2 and VNP-2-PIF6 on nickel columns loaded with PhyB651-His6 or PhyB917-His₆. “**” means the p-value <0.01.

FIG. 7C depicts the capture efficiency under red light conditions for various column loadings of PhyB917-His₆ using VNP-2-PIF6.

FIG. 8A depicts an experimental strategy for confirming binding of VNP-2-PIF6 to PhyB917 and dissociation upon exposure to far red light. Figure discloses “His₆” as SEQ ID NO: 23.

FIG. 8B depicts the capture efficiency for eluted VNP-2-PIF6 bound to PhyB917-His₆ that is exposed to far red light after elution (FR reversed) or kept under red light (R Only, control) based on the strategy depicted in FIG. 8A.

FIG. 8C depicts the capture efficiency for PhyB917-His₆ and PhyB917(Y276)H-His₆ at varying column loadings under red light conditions.

FIG. 9A depicts a mechanism for decreasing or increasing nuclear uptake of a virus displaying an optogenetic binding partner (PIF6) on its surface into a target cell where an optogenetic protein (PhyB) and its associated chromophore are present to form the holoprotein (Pr and Pfr) in the cytoplasm, the optogenetic protein having a nuclear localization signal (NLS) on its surface and exposing the system to far-red (inactivating) light or red (activating light) to decrease or enhance nuclear uptake of the virus, respectively.

FIG. 9B depicts HeLa cell nuclei stained with Hoescht nuclear stain (“Nucleus”) after exposure to VNP-2-PIF6 under red (650 nm) or far red (730 nm) light, immunofluorescence of VNP-2-PIF6 in the cells (“VNP-PIF6”) and the co-localized image of VNP-2-PIF6 in cell nuclei (“Colocalized”). Scale bar=20 μm.

FIG. 9C depicts HeLa cell nuclei of cells expressing PhyB908 stained with Hoescht nuclear stain (“Nucleus”) after exposure to VNP-2-PIF6 under red (650 nm) or far red (730 nm) light, immunofluorescence of VNP-2-PIF6 in the cells (“VNP-PIF6”) and the co-localized image of VNP-2-PIF6 in cell nuclei (“Colocalized”). Scale bar=20 μm.

FIG. 9D depicts HeLa cell nuclei of cells expressing PhyB908-NLS stained with Hoescht nuclear stain (“Nucleus”) after exposure to VNP-2-PIF6 under red (650 nm) or far red (730 nm) light, immunofluorescence of VNP-2-PIF6 in the cells (“VNP-PIF6”) and the co-localized image of VNP-2-PIF6 in cell nuclei (“Colocalized”). Scale bar=20 μm.

FIG. 9E depicts the Pearson Correlation Coefficient for the images analyzed for the negative control (Neg.), PhyB908 (PhyB) and PhyB908-NLS (PhyB-NLS) cells under red (R) and far red (FR) light conditions. ** indicates statistical significance of the value (p-value <0.001).

FIG. 9F depicts HeLa cell nuclei of cells expressing PhyB650-NLS stained with Hoescht nuclear stain (“Nucleus”) after exposure to VNP-2-PIF6 under red (650 nm) or far red (730 nm) light or wtAAV2, immunofluorescence of VNP-2-PIF6 in the cells (“VNP-PIF6”) and the co-localized image of VNP-2-PIF6 in cell nuclei (“Colocalized”).

FIG. 10A depicts an orthoptic nuclear slice along x-, y- and z-axes, focused on the location indicated by the crosshairs in cells that have been transduced with VNP-2-PIF6 at a MOI of 5,000 without expression of PhyB908 (left image), with expression of PhyB908-NLS under far red light conditions (middle image) or with expression of PhyB908-NLS under red light conditions (right image). Scale bar=10 μm.

FIG. 10B depicts the y-axis cross-section showing cells that have been transduced with VNP-2-PIF6 at a MOI of 5,000 without expression of PhyB908 or with expression of PhyB908-NLS under far red light or red light conditions showing Hoechst and A20 signal (left images) or only A20 signal (right images). Scale bar=4 μm.

FIG. 11A depicts an apparatus for applying R and FR light via LEDs to a tissue culture well with a glass bottom for control the R:FR light ratio.

FIG. 11B depicts the % of cells expressing GFP in HeLa cells expressing PhyB908 or PhyB908-NLS transduced by VNP-2-PIF6 at 24 hours post-transduction. Cells were exposed to different intensities (μmol/m²s) of red and far red light as shown on the x-axis.

FIG. 11C depicts the transduction index cells expressing GFP in HeLa cells expressing PhyB908 or PhyB908-NLS transduced by VNP-2-PIF6 at 24 hours post-transduction. Cells were exposed to different intensities of red and far red light as shown on the x-axis.

FIG. 11D depicts the % of cells expressing GFP in HeLa cells expressing PhyB908 or PhyB908-NLS transduced by VNP-2-PIF6 or wtAAV2 at 48 hours post-transduction. Cells were exposed to different intensities of red and far red light as shown on the x-axis.

FIG. 11E depicts the transduction index cells expressing GFP in HeLa cells expressing PhyB908 or PhyB908-NLS transduced by VNP-2-PIF6 or wtAAV2 at 48 hours post-transduction. Cells were exposed to different intensities of red and far red light as shown on the x-axis.

FIG. 11F depicts fluorescent micrographs of GFP expression in HeLa cells constitutively expressing PhyB-NLS and treated with or without VNP-2-PIF6, PCB, and red light.

FIG. 11G depicts the discrete transfer functions for transduction by VNP-2-PIF6 in HeLa cells under increasing red light flux between 0 and 10 μM/m²s.

FIG. 11H depicts the full-range logarithmic transfer function of transduction index by VNP-2-PIF6 facilitated by PhyB908-NLS under varying R:FR ratios. Each data point is the average of 4-5 replicates from 2 independent experiments.

FIG. 12 depicts the fold change in transduction index for hMSC, HUVEC and 3T3 cells constitutively expressing PhyB908-NLS and exposed to VNP-2-PIF6 for 48 hours under red (R) light or far red (FR) light.

FIG. 13A depicts the transduction index as a function of red light intensity for a fixed intensity of FR light.

FIG. 13B depicts the transduction index at maximum far red light intensity only (15 μM/M²s) and maximum red light intensity only (43 μM/m²s).

FIG. 14 depicts spatial patterning of GFP expression in HeLa cells using photomasks and either red light only or co-delivery of red and far red light. The photomask patterns are shown below each corresponding image. Scale bar=2 mm.

FIG. 15A depicts the transduction index for an AAV virus comprising VP1 and VP3 in the viral capsid, having on its capsid surface, embedded in VP1, the LOV domain from Avena sativa phototropin 1 protein with a N-terminal Pkit nuclear export signal and a C-terminal nuclear localization signal as wells an enzymatic cleavage motif (DDDDK) susceptible to cleavage by enterokinase, with or without pre-treatment with enterokinase prior to the transduction and in the presence of varying intensities of blue light.

FIG. 15B depicts a Western blot of wild-type AAV and the virus used in FIG. 15A with or without enterokinase (SEQ ID NO: 76) treatment.

### DESCRIPTION

The present invention will be described with respect to particular embodiments and with reference to certain drawings, but the invention is not limited thereto. The drawings described are only schematic and are non-limiting. In the drawings, the size of some of the elements may be exaggerated or distorted and not drawn on scale for illustrative purposes. Where the elements of the invention are designated as “a” or “an” in first appearance and designated as “the” or “said” for second or subsequent appearances unless something else is specifically stated.

The present invention now will be described more fully here with reference to the accompanying drawings, in which some, but not all embodiments of the invention are shown. Indeed, the invention may be embodied in many different forms and should not be construed as limited to the embodiments set forth herein, rather, these embodiments are provided so that this disclosure satisfies all the legal requirements.

The present disclosure provides compositions and methods using optogenetic tools to provide tunable spatial and temporal control of gene delivery using viral vectors.

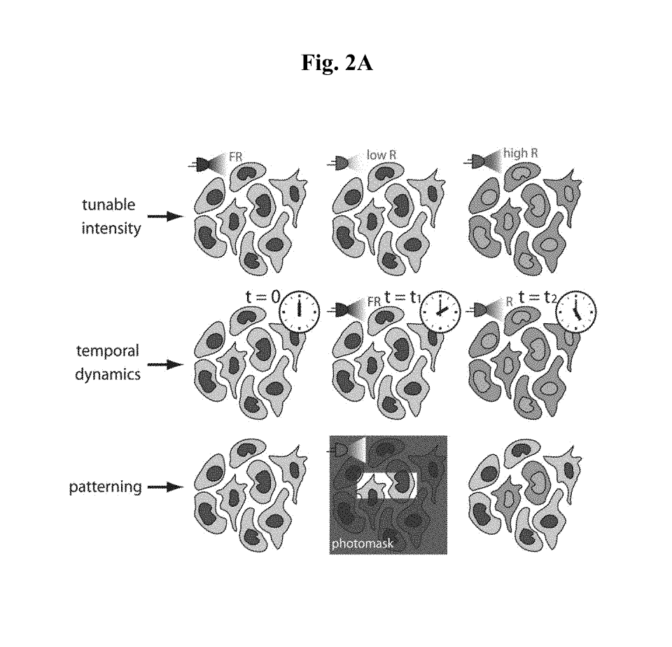

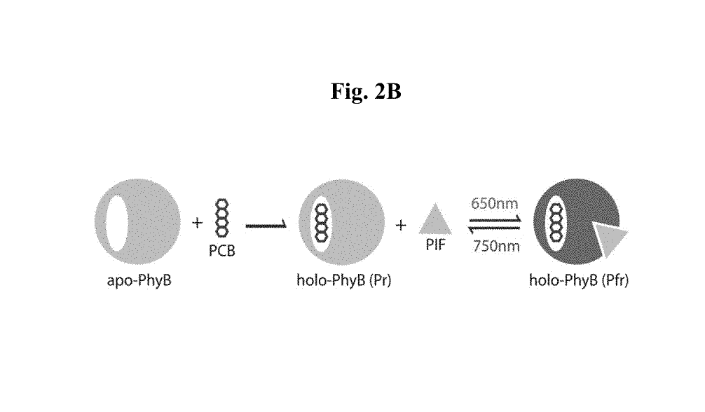

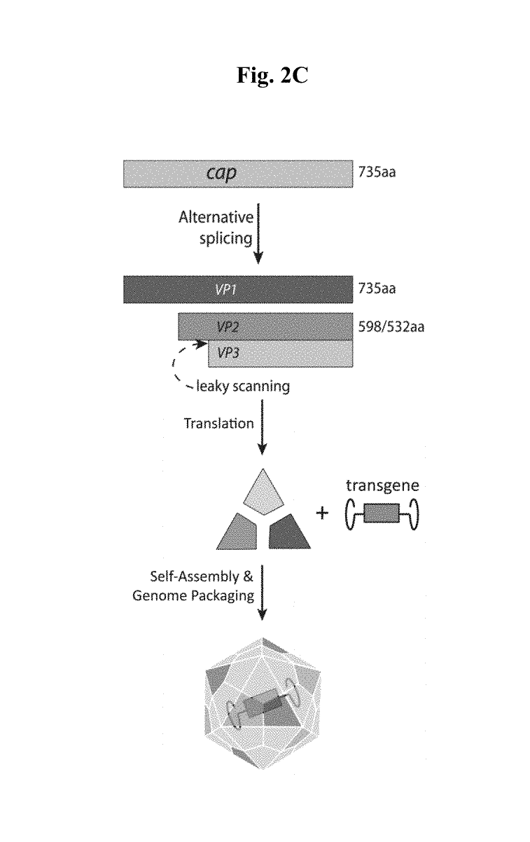

In some embodiments, as shown in FIG. 2A, gene delivery in a cell population can be controlled to deliver analog levels of expression using activating, e.g. “low R” or “high R”, light versus deactivating, e.g. “FR”, light while the cells are exposed to a light-activable viral vector. Using activating light, expression can be tuned by altering the intensity of the light, e.g. “low R” versus “high R”, as shown in the top row of FIG. 2A (“tunable intensity”). The medium shading in the cells in the middle panel reflect a lower level of expression while the darker shading in the cells in the right panel reflect a higher level of expression. Light-activable gene delivery can also be controlled by the timing of introduction of activating, e.g. “R”, light as shown in the middle row of FIG. 2A (“temporal dynamics”) where the cell population, in the presence of the viral vector, is exposed to deactivating “FR” light until such time as activation is desired and the cells are exposed to activating light. Because light can also be controlled spatially, through the use of photomasks, the expression in a cell population can be spatially patterned by placing a photomask over the cell population, which is exposed to the viral vector, while exposed to activating light as shown in the bottom row of FIG. 2A (“patterning”).

Unless otherwise defined, all technical and scientific terms used herein have the same meaning as commonly understood by one of ordinary skill in the art.

As used herein, the term “optogenetic protein” means an amino acid sequence that changes its conformation (e.g. tertiary structure) in response to light of certain wavelengths or ranges of wavelengths. For example, Phytochrome B (PhyB) (SEQ ID NO: 126) adopts a first conformation when exposed to red light (λₘₐₓ=650 nm) and adopts a second conformation when exposed to far red light (λₘₐₓ=750 nm).

The term “optogenetic binding partner” as used herein means an amino acid sequence capable of binding to an optogenetic protein in at least some conformations of the optogenetic protein. The optogenetic binding partner is capable of binding to an optogenetic protein when the optogenetic protein is in a first conformation but is not capable of binding to the optogenetic protein when the optogenetic protein is in a second conformation. For example, PIF6 (SEQ ID NO: 140) can reversibly bind to PhyB; when PhyB is exposed to red light, PIF6 binds to PhyB, however, when PhyB is exposed to far red light, PIF6 cannot bind PhyB and dissociates from PhyB due to the conformational change of PhyB in response to the wavelengths of light. FIG. 2B shows the covalent association of apo-PhyB with its chromophore (PCB) to yield the photoresponsive holoprotein (“holo-PhyB (Pr)”) which can then associate (to form “holo-PhyB (Pfr)”) or dissociate from its binding partner, PIF6 (“PIF”), upon exposure to activating red (650 nm) or deactivating far red (750 nm) light, respectively.

As it relates to amino acid sequence location, a first amino acid sequence is considered adjacent to a second amino acid sequence if it is located outside of the second amino acid sequence and is located at the N- or C-terminus of the second amino acid sequence. Two amino acid sequences are adjacent even when intervening sequences, such as linkers, are present between the amino acid sequences. Similarly, as it relates to nucleic acid sequence location, a first nucleic acid sequence is considered adjacent to a second nucleic acid sequence if it is located outside of the second nucleic acid sequence and is located at the 5′ end or the 3′ end of the second nucleic acid sequence. Two nucleic acid sequence are adjacent even when intervening sequences, such as linker sequences, are present between the nucleic acid sequences.

As it relates to amino acid sequence location, a first amino acid sequence is considered embedded within a second amino acid sequence if it is located such that a first portion of the second amino acid sequence is located adjacent to one end (N-terminal or C-terminal) of the first amino acid sequence and a second portion of the second amino acid sequence is adjacent to the opposite end of the first amino acid sequence.

Throughout this disclosure, the terms peptide and protein and peptides and proteins are used interchangeably unless otherwise noted. Portions and variants of proteins recited herein are to be understood to retain the type of activity of the reference protein, although the activity may be lesser or greater than that of the reference protein.

It should be understood, that throughout this disclosure the reference to nucleic acids includes any nucleic acid, such as, by way of example but not limitation, DNA, RNA, cDNA. In some embodiments, a nucleic acid molecule is a cDNA, DNA or RNA molecule.

The present disclosure also provides for genetic insertion of small peptides or proteins into any AAV capsid such that the peptide or protein is attached at only one end to the virus capsid. The peptides are presented on the capsid surface in an unconstrained conformation, in some cases linear, via enzymatic digestion, which relieves any conformational tension the peptide would otherwise experience being anchored at two ends. Thus, a prototype virus with peptide “locks” that are protease-susceptible and are displayed as linear substrates on the AAV capsid is provided. The peptide locks can initially prevent the virus' interactions with cells to prevent uptake and transduction or limit the activity of the inserted protein or other viral processes. Proteases upregulated in diseased sites can remove these locks to allow subsequent virus transduction and gene delivery. Alternatively, the AAV can be subjected to proteases prior to exposure to a target cell or prior to administration to a subject for gene therapy. In some instances, pre-treatment with a protease to cleave an enzymatic cleavage motif can be combined with administration of the virus to diseased tissue where it can be cleaved by another protease, e.g. a MMP.

Such viruses are useful for cell targeting and/or stimulus-responsive drug/gene delivery application where peptides or proteins need to be displayed on the AAV capsid in a non-conformationally constrained fashion. Typically, genetic insertion of peptides in the middle of AAV capsid proteins requires both ends of the peptide/protein to remain attached to the capsid protein. In order for the inserted peptides to interact with target partners/enzymes, it is important for the inserted peptide to adopt its natural conformation upon insertion into the AAV capsid, which is provided by the present disclosure.

In an embodiment, a virus is provided which includes a capsid protein and an optogenetic binding partner, wherein at least a portion of the optogenetic binding partner is displayed on the surface of the virus, and wherein the optogenetic binding partner is linked to the capsid protein by a direct amino acid linkage or a linker.

In an embodiment, a virus is provided which includes a capsid protein and an optogenetic protein, wherein at least a portion of the optogenetic protein is displayed on the surface of the virus and wherein the optogenetic protein is linked to the capsid protein by a direct amino acid linkage or a linker.

In some embodiments, the virus which includes a capsid protein and an optogenetic binding partner can further include an enzymatic cleavage motif adjacent to the optogenetic binding partner, wherein the enzymatic cleavage motif does not inactivate other biologically active motifs on the surface of the virus.

In some embodiments, the virus which includes a capsid protein and an optogenetic protein can further include an enzymatic cleavage motif adjacent to the optogenetic protein, wherein the enzymatic cleavage motif does not inactivate other biologically active motifs on the surface of the virus.

In an embodiment, an amino acid molecule is provided which includes a capsid protein and an optogenetic binding partner, wherein at least a portion of the optogenetic binding partner is displayed on the surface of the capsid protein, and wherein the optogenetic binding partner is linked to the capsid protein by a direct amino acid linkage or a linker.

In an embodiment, an amino acid molecule is provided which includes a capsid protein and an optogenetic protein, wherein at least a portion of the optogenetic protein is displayed on the surface of the capsid protein, and wherein the optogenetic protein is linked to the capsid protein by a direct amino acid linkage or a linker.

In some embodiments, the amino acid molecule which includes a capsid protein and an optogenetic binding partner can further include an enzymatic cleavage motif adjacent to the optogenetic binding partner.

In some embodiments, the amino acid molecule which includes a capsid protein and an optogenetic protein can further include an enzymatic cleavage motif adjacent to the optogenetic protein.

In an embodiment, a nucleic acid molecule is provided which encodes a capsid protein of a virus and an optogenetic binding partner that is linked to the capsid protein by at least one amino acid linkage or linker, and wherein at least a portion of the optogenetic binding partner is displayed on the surface of the capsid protein.

In an embodiment, a nucleic acid molecule is provided which encodes a capsid protein of a virus and an optogenetic protein that is linked to the capsid protein by at least one amino acid linkage or linker, and wherein the optogenetic protein is displayed on the surface of the capsid protein.

In some embodiments, the nucleic acid molecule which encodes a capsid protein and an optogenetic binding partner can further encode an enzymatic cleavage sequence which encodes an enzymatic cleavage motif adjacent to the optogenetic binding partner.

In some embodiments, the amino acid molecule which encodes a capsid protein and an optogenetic protein can further encode an enzymatic cleavage sequence which encodes an an enzymatic cleavage motif adjacent to the optogenetic protein.

In some embodiments, a method for delivering a nucleic acid molecule to the nucleus of a target cell includes the steps of obtaining a virus as described in the present disclosure having an optogenetic protein on the capsid surface with a nuclear localization signal; delivering the virus to the target cell; and exposing the target cell to a light of a sufficient wavelength to induce a conformational change in the optogenetic protein that exposes the nuclear localization signal, resulting in enhancement of the delivery of the nucleic acid molecule to the nucleus of the target cell as compared to without exposure to the light of a sufficient wavelength to induce a conformational change in the optogenetic protein.

In some embodiments, a method for delivering a nucleic acid molecule to the nucleus of a target cell includes the steps of obtaining a virus as described in the present disclosure having an optogenetic binding partner on the capsid surface; delivering the virus to a target cell containing an optogenetic protein which further comprises a nuclear localization signal and which is capable of binding the optogenetic binding partner, portion thereof or variant thereof present on the surface of the virus; and exposing the target cell to light of a sufficient wavelength to induce a conformational change in the optogenetic protein that allows the optogenetic protein to bind to the optogenetic binding partner, portion thereof or variant thereof present on the surface of the virus, thereby enhancing nuclear delivery of the virus.

In some embodiments, a method for delivering a nucleic acid molecule to the nucleus of a target cell includes the steps of obtaining a virus with an optogenetic protein displayed on its surface, delivering the virus to a target cell containing an optogenetic binding partner capable of binding to the optogenetic protein and having a nuclear localization signal, and exposing the target cell to light of a sufficient wavelength to induce a conformational change in the optogenetic protein to allow binding of the optogenetic protein and the optogenetic binding partner, enhancing delivery of the virus.

The foregoing methods may be modified to enhance or decrease the nuclear delivery of a nucleic acid molecule to the nucleus of a target cell by incorporating a nuclear localization signal or nuclear export signal as described further herein and/or by using activating and de-activing wavelengths of light for the respective optogenetic protein as described further herein. In some instances, the virus and/or capsid protein can further include an enzymatic cleavage motif, cleavable by an enzyme, and the virus can be pre-treated with the enzyme to further expose and/or allow the inserted protein—e.g. optogenetic protein or optogenetic binding partner—to adopt a more thermodynamically favorable conformation and enhance transduction efficiency.

In some embodiments, a kit is provide which includes a virus or nucleic acid molecule as described in the present disclosure for preparing at least a portion of the virus, where the virus has an enzymatic cleavage motif inserted into the capsid protein, and a protease for pre-treating the virus prior to use to expose a protein inserted into the capsid protein.

Viruses and Capsid Proteins

Viral capsid proteins encapsidate the genetic material of viruses. For example, the capsid of AAV comprises three distinct capsid subunit types, designated VP1, VP2 and VP3.

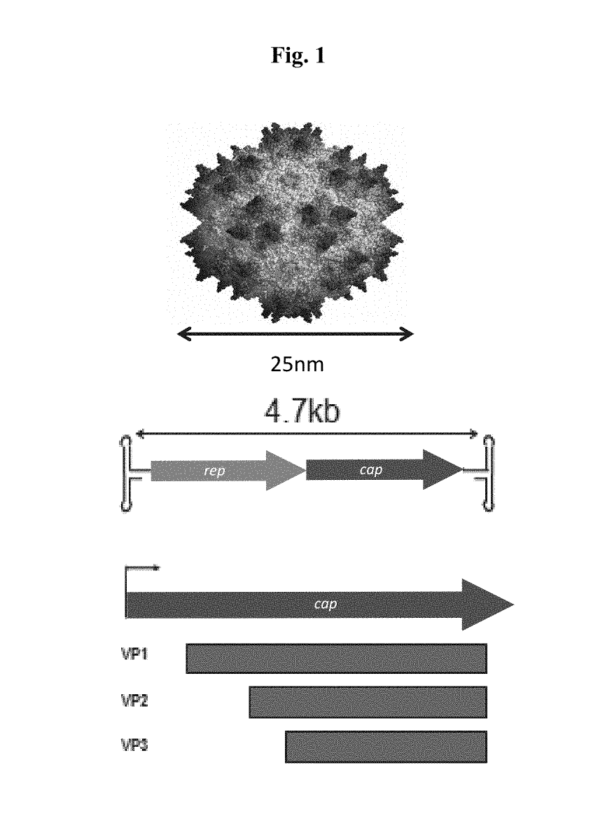

AAV is a 25 nm, non-enveloped virus. As shown in FIG. 1, the intact AAV virus capsid, which contains the 4.7 kB genome of AAV which includes the rep and cap genes is comprised of VP1, VP2 and VP3 which are variants produced from the same cap ORF. These three viral proteins—VP1, VP2 and V3—assemble together in a 1:1:10 ratio to form a 60-mer shell of AAV. The single-stranded DNA genome of AAV is carried within the capsid lumen. As shown in FIG. 2C, in wild-type AAV, the capsid subunits (VP1, VP2 and VP3) are produced from the same cap ORF by alternate mRNA splicing and alternative translation start codon usage. For AAV2, VP1 (SEQ ID NO: 50, nucleotide sequence at SEQ ID NO: 49) is a 735aa protein, and VP2 and VP3 (SEQ ID NO: 52 and SEQ ID NO: 54, respectively, nucleotide sequences at SEQ ID NOs: 51 and 53, respectively) are truncated alternative splice variants of VP1 missing the N-terminal 137 or 203aa, respectively. Because the VP1, VP2 and VP3 subunits of AAV can self-assemble, in a ratio of 1:1:10 respectively, to form the viral capsid, the addition of a transgene of interest or other genetic material permits the inclusion of the transgene or other genetic material into the capsid structure upon self-assembly of the capsid subunits. AAV naturally infects human cells with a relatively high efficiency with an absence of pathological effects associated with its infection, which has led to its widespread testing for gene delivery applications. AAV can infect both dividing and non-dividing cells and persist in an extrachromosomal state without integrating into the genome of the host cell. The AAV capsid is amenable to insertion of proteins and peptides, although the size and location of insertion may be limited due to effects on viral capsid formation and other considerations.

In embodiments of the present invention, any virus capable of delivering genetic material to a target cell may be used. In some embodiments, the virus is AAV. In certain embodiments, the virus is AAV of serotype 2 (AAV2). Different AAV serotypes, such as AAV of any of serotypes 1-12 (nucleotide sequences SEQ ID NO: 79, 82, 85, 88, 91, 94, 97, 100, 103, 104, 106 and 108 corresponding to serotypes 1, 2, 3, 4, 5, 6, 7, 8, 9, 10, 11, and 12, respectively), can be used and have varying tissue tropism. This varying tissue tropism, coupled with the light-activation of the present invention can permit for defined gene expression profiles in living animals in terms of spatial distribution and overall efficiency.

The rep genes of AAV viruses of serotypes 1, 2, 3, 4, 5, 6, 7, 8 and 12 can be found at SEQ ID NOs: 80, 83, 86, 89, 92, 95, 98, 101 and 109, respectively. The cap genes of AAV viruses of seroptypes 1, 2, 3, 4, 5, 6, 7, 8, 10, 11 and 12 can be found at SEQ ID NOs: 81, 84, 87, 90, 93, 96, 99, 102, 105, 107 and 110, respectively.

In addition, the capsid proteins useful in the present disclosure may vary according to the type of virus and the tolerance of the individual capsid proteins for insertion of peptide sequences. In some embodiments, the virus is an AAV of any of serotypes 1-12 (nucleotide sequences SEQ ID NO: 79, 82, 85, 88, 91, 94, 97, 100, 103, 104, 106 and 108, respectively). In some embodiments, where the virus is AAV2, the capsid protein may be VP1 (SEQ ID NO: 50, nucleotide sequence at SEQ ID NO: 49), VP2 (SEQ ID NO: 52, nucleotide sequence at SEQ ID NO: 51), VP3 (SEQ ID NO: 54, nucleotide sequence at SEQ ID NO: 53), portions thereof, variants thereof and combinations thereof. In certain embodiments, the capsid protein is VP1. In some embodiments, the capsid protein is VP2. In certain embodiments, the capsid protein is VP3.

The nucleotide sequence of a nucleic acid encoding the capsid protein can encode the nucleotide sequence of VP1, VP2, VP3, portions thereof, variants thereof and combinations thereof.

Optogenetic Binding Partners and Optogenetic Proteins

Optogenetic binding partners and optogenetic proteins include a broad class of proteins which can interact under varying light conditions. In embodiments of the present invention, the optogenetic binding partner can be any amino acid sequence capable of binding to an optogenetic protein in at least some conformations of the optogenetic protein. For example, PIF6 can bind to PhyB under red light but cannot bind to PhyB and dissociates from PhyB, if bound, under far red light. Specific optogenetic binding partners that can be used in embodiments of the present invention include, by way of example but not limitation, PIF1 (SEQ ID NO: 136 (nucleotide)), PIF2, PIF3, PIF4 (SEQ ID NO: 137 (nucleotide)), PIF5 (SEQ ID NO: 139 (nucleotide)) and PIF6 (SEQ ID NO: 140). In some embodiments, the optogenetic binding partner is PIF6, a portion thereof or a variant thereof. In some embodiments, the optogenetic binding partner comprises the first 100 amino acids of PIF6 (SEQ ID NO: 121, nucleotide sequence at SEQ ID NO: 120). In some embodiments, the portion of PIF6 can also be SEQ ID NO: 48 (nucleotide SEQ ID NO: 47). In some embodiments, the optogenetic binding partner, portion thereof or variant thereof is embedded within the amino acid sequence of the capsid protein. In other embodiments, the optogenetic binding partner, portion thereof or variant thereof is adjacent to the amino acid sequence of the capsid protein.

In embodiments of the present invention that include an optogenetic protein, the optogenetic protein can be any amino acid sequence that changes its conformation in response to light of certain wavelengths or ranges of wavelengths. For example, PhyB adopts a first conformation when exposed to red light and adopts a second conformation when exposed to far red light. Types of optogenetic proteins that can be used in embodiments of the present invention include, by way of example but not limitation, phytochromes, light-oxygen-voltage (LOV) proteins, portions thereof and variants thereof. In some embodiments, the optogenetic protein is PhyB or a variant thereof. In certain embodiments, the optogenetic protein is the LOV domain from Avena sativa phototropin 1 protein or a variant thereof. In some embodiments, the optogenetic protein can be at least a portion or variant of PhyB (SEQ ID NO: 126), the LOV domain from Avena sativa phototropin 1 protein (SEQ ID NO: 68, nucleotide SEQ ID NO: 67), Dronpa (SEQ ID NO: 112, nucleotide SEQ ID NO: 111) or Cry2 (encoded by nucleotide SEQ ID NO: 113). The properties of these optogenetic proteins are shown in Table 1 below.

TABLE I
Exemplary Optogenetic Proteins and Their Properties

| Protein | ON λ | Size (aa) | Chromophore | Parts | Photo-response |
|---|---|---|---|---|---|
| PhyB | 650 | 450 | PCB | 3 | Heterodimerization, |
| | | | | | divalent |
| LOV | 450 | 144 | FMN | 1 | Reveals blocked |
| | | | | | domain |
| Dronpa | 500 | 210 | none | 1 | Homodimerization, |
| | | | | | multivalent; |
| | | | | | fluorescent |
| Cry2 | 400 | 350 | FAD | 2 | Heterodimerization, |
| | | | | | divalent |

In some embodiments, the optogenetic protein is embedded within the amino acid sequence of the capsid protein. In other embodiments, the optogenetic protein is adjacent to the amino acid sequence of the capsid protein.

In some embodiments, where the virus is AAV2 and the capsid protein comprises VP2, the optogenetic binding partner or optogenetic protein can be adjacent to the N-terminus of the amino acid sequence of VP2 or inserted at G316 in the amino acid sequence of SEQ ID NO: 52 (VP2). In an embodiment, the virus and/or amino acid molecule comprises or the nucleic acid molecule encodes the amino acid sequence of SEQ ID NO: 46 (VNP-2-PIF6) (nucleotide sequence at SEQ ID NO: 45). In certain embodiments, where the virus is AAV and the capsid protein comprises VP1, the optogenetic binding partner or optogenetic protein can be inserted at M138 or G453 of SEQ ID NO: 50 (VP1). In some embodiments, the virus and/or amino acid molecule comprises, or the nucleic acid encodes, the amino acid sequence of SEQ ID NO: 44 (VNP-1-PIF6) (nucleotide sequence at SEQ ID NO: 43). In certain embodiments, the virus and/or amino acid molecule comprises, or the nucleic acid encodes, the amino acid sequence encoded by SEQ ID NO: 114 (VNP-1,2-PIF6). In some embodiments, the virus and/or amino acid molecule comprises, or the nucleic acid molecule encodes, the amino acid sequence of SEQ ID NO: 54 (VP3). In certain embodiments, the optogenetic binding partner or optogenetic protein is inserted at G250 in amino acid sequence of SEQ ID NO: 54 (VP3). The site of insertion can vary based on the size of the insert and the tolerance of the virus and/or capsid of such insertion.

In any of the embodiments described herein, the number of optogenetic proteins or optogenetic binding partners displayed per virus capsid can be varied. Optogenetic proteins and optogenetic binding partners can be displayed on all subunits or just a subset of subunits. Mutants of the optogenetic proteins and optogenetic binding partners can also be used to modulate the functional properties of the system.

Linkers

In some embodiments, a virus or amino acid molecule can further comprise at least one linker between the amino acid sequence of the optogenetic binding partner or optogenetic protein and the capsid protein. A linker is any amino acid sequence that lies between a first amino acid sequence a second amino acid sequence, thus linking the two sequences. A preferred linker is GGS and can also be incorporated as (GGS)ₙ or GₙS where n is an integer number and denotes the number of GGS sequences or G residues in the linker, respectively. Linker sequences can also include, by way of example but not limitation, AG, GA, G or GGGS (SEQ ID NO: 4). n can be any integer value and can, by way of example but not limitation, be 1, 2, 3, 4, 5, 6, 7, 8, 9 or 10.

In certain embodiments, the virus or amino acid molecule further comprises at least one linker between the N-terminus of the amino acid sequence of the optogenetic binding partner and the amino acid sequence of the capsid protein or between the C-terminus of the amino acid sequence of the optogenetic binding partner and the amino acid sequence of the capsid protein. In some embodiments, the virus or amino acid molecule further comprises a first linker between the N-terminus of the amino acid sequence of the optogenetic binding partner and the amino acid sequence of the capsid protein and a second linker between the C-terminus of the amino acid sequence of the optogenetic binding partner and the amino acid sequence of the capsid protein.

In certain embodiments, the virus or amino acid molecule further comprises at least one linker between the N-terminus of the amino acid sequence of the optogenetic protein, portion thereof or variant thereof and the amino acid sequence of the capsid protein or between the C-terminus of the amino acid sequence of the optogenetic protein and the amino acid sequence of the capsid protein. In some embodiments, the virus or amino acid molecule further comprises a first linker between the N-terminus of the amino acid sequence of the optogenetic protein and the amino acid sequence of the capsid protein and a second linker between the C-terminus of the amino acid sequence of the optogenetic protein and the amino acid sequence of the capsid protein.

In some embodiments, the nucleic acid molecule encodes at least one linker between the N-terminus of the amino acid sequence of the optogenetic binding partner and the amino acid sequence of the capsid protein or between the C-terminus of the amino acid sequence of the optogenetic binding partner and the amino acid sequence of the capsid protein. In some embodiments, the nucleic acid molecule further encodes a first linker between the N-terminus of the amino acid sequence of the optogenetic binding partner and the amino acid sequence of the capsid protein and a second linker between the C-terminus of the amino acid sequence of the optogenetic binding partner and the amino acid sequence of the capsid protein.

In certain embodiments, the nucleic acid molecule encodes at least one linker between the N-terminus of the amino acid sequence of the optogenetic protein and the amino acid sequence of the capsid protein or between the C-terminus of the amino acid sequence of the optogenetic protein and the amino acid sequence of the capsid protein. In some embodiments, the nucleic acid molecule further encodes first linker between the N-terminus of the amino acid sequence of the optogenetic protein and the amino acid sequence of the capsid protein and a second linker between the C-terminus of the amino acid sequence of the optogenetic protein and the amino acid sequence of the capsid protein.

Nucleic Acid Molecules for Delivery

In any of viral embodiments of the present invention, the virus can further include a nucleic acid molecule. In certain embodiments, the nucleic acid molecule can be a therapeutic nucleic acid molecule. In some embodiments, the therapeutic nucleic acid molecule is selected from the group consisting a gene, a portion of a gene, RNA interference and a CRISPR/Cas genome editing tool. It may be understood that any nucleic acid desired to be delivered to a target cell can be used in the virus.

Nuclear Localizations Signals and Nuclear Export Signals

In some embodiments, a nuclear localization signal (NLS) can be incorporated on the surface of the capsid protein or on the optogenetic protein, portion thereof or variant thereof. In some embodiments, the NLS is not exposed when the optogenetic protein is in a first configuration and is exposed when the optogenetic protein is in a second configuration. In this way, using the light-responsive properties of the optogenetic protein, the exposure—and activity—of the NLS can be regulated to increase or decrease nuclear uptake. In certain embodiments, a nuclear export signal (NES) can be incorporated on the surface of the capsid protein or the optogenetic protein.

Suitable NLS can include, by way of example not limitation, PKKKRKV (SEQ ID NO: 5) or TRPQRDCPTPTWQPQPRRKSW (SEQ ID NO: 6). Other suitable NLS include, by way of example but not limitation, SEQ ID: 143 to SEQ ID: 172. Suitable NES can include, by way of example, but not limitation, LQLPPLERLTL (SEQ ID NO: 7), LPPLERLTL (SEQ ID NO: 8), PSTRIQQQLGQLTLENLQ (SEQ ID NO: 9), or MLALKLAGLDI (SEQ ID NO: 10). Additional nuclear export signals can include, by way of example but not limitation, NLVDLQKKLEELELDEQQ (SEQ ID NO: 174) and LALKLAGLDIGGSGGSLALKLAGLDI (SEQ ID NO: 175). In some embodiments, a nucleic acid molecule encoding a NLS can include, by way of example but not limitation, the nucleotide sequence(s) CCCAAGAAAAAGCGGAAGGTG (SEQ ID NO: 11) or ACGAGGCCGCAAAGAGACTGCCCGACGCCAACCTGGCAGCCGCAGCCAAGAAGAA AAAGCTGGAC (SEQ ID NO: 12). In some embodiments, a nucleic acid molecule encoding a NES can include, by way of example but not limitation, the nucleotide sequence(s) CTTCAACTTCCTCCTCTTGAGAGACTTACTCTT (SEQ ID NO: 13), CTTCCTCCTCTTGAGAGACTTACTCTT (SEQ ID NO: 14), CCCAGCACCCGGATCCAGCAGCAGCTGGGCCAGCTGACCCTGGAGAACCTGCAG (SEQ ID NO: 15), or ATGTTAGCCTTGAAATTAGCAGGTCTTGATATC (SEQ ID NO: 16).

In some embodiments, the NES is present on the surface of the capsid protein or on the optogenetic protein. In some embodiments, the NES is not exposed when the optogenetic protein is in a first configuration and is exposed when the optogenetic protein is in a second configuration. In this way, using the light-responsive properties of the optogenetic protein, the exposure—and activity—of the NES can be regulated to increase or decrease nuclear uptake. In some embodiments, both a NLS and a NES are present on the capsid protein or optogenetic protein. This can help to limit background/basal levels of transduction.

Enzymatic Cleavage Motifs

As used herein, an “enzymatic cleavage motif” is an amino acid sequence that is susceptible to cleavage by a protease. In certain embodiments, the protease is a matrix metalloprotease (MMP) or endopeptidase. In some embodiments, the protease is an endopeptidase. The protease can be any protease which cleaves a known amino acid sequence, such as proteases used to cleave known purification tags. The protease can, by way of example but not limitation, be a matrix matalloproeinase (MMP), an endopeptidase, a kinase, TEV protease, Cathepsin K (CTSK), a phosphatase and combinations thereof.

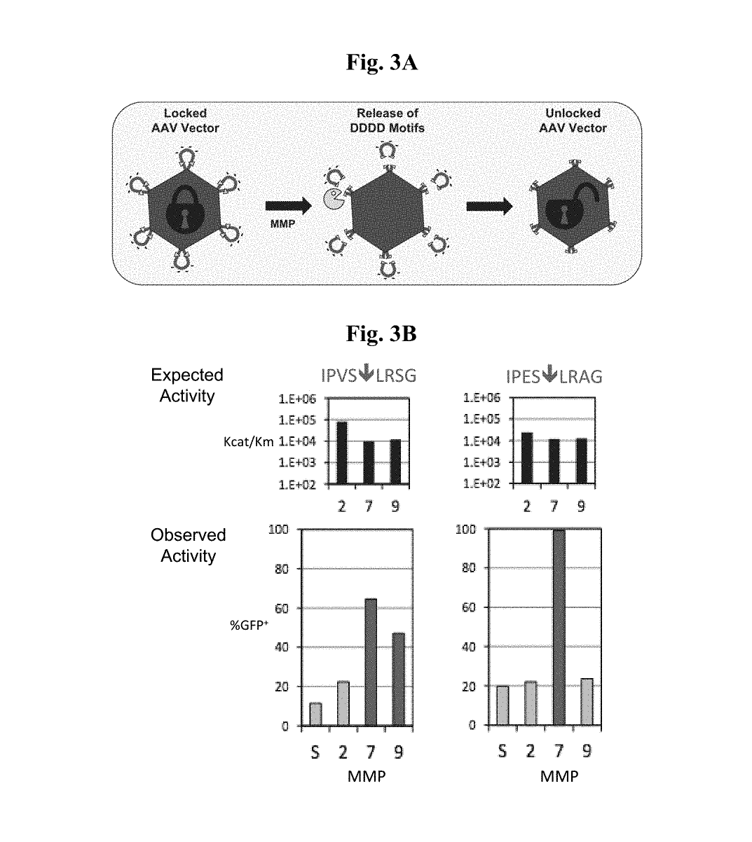

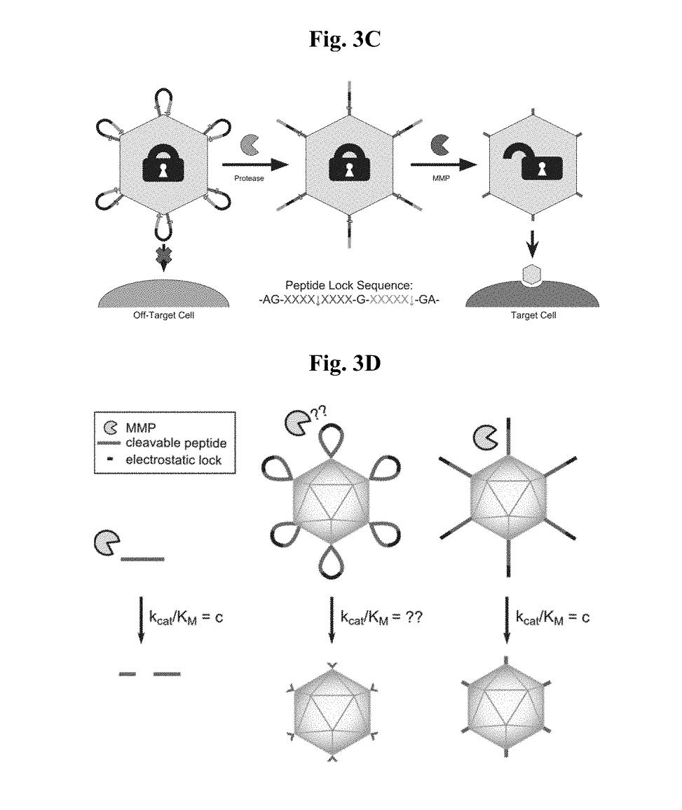

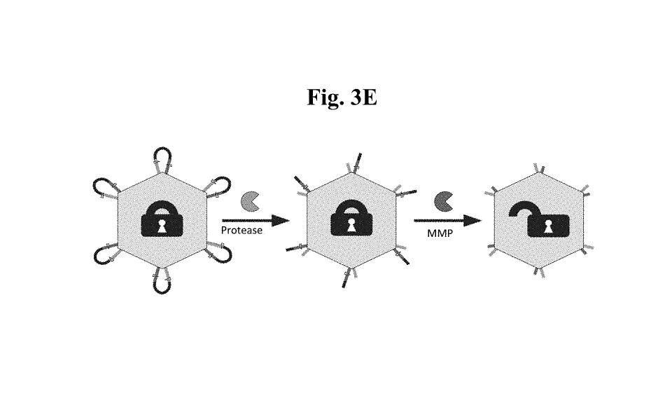

As shown in FIG. 3A, conventional peptide locks can be used to lock an adeno-associated virus-based vector by blocking binding with the cell surface receptor, thereby preventing infection. The lock is flanked by two MMP-cleavable sequences, so that in the presence of MMPs, the lock is cleaved off, unlocking the vector and allowing it to resume transduction. FIG. 3B shows the expected activity, expressed as k_{cat}/k_{M}, for MMP-cleavable peptide locks with two cleavage sites for the same MMP with MMP-2, MMP-7 and MMP-9, versus the observed activity as % GFP⁺ cells after infection. As shown, the observed activity does not correlate with the expected activity, potentially due to steric effects due to the presence of two “locked” cleavage sites.

FIG. 3C shows an embodiment of the present invention where the peptide lock functions similarly to block cell binding but, instead of two of the same cleavage site, contains two cleavage sequences, one recognized by protease and one by MMPs. Prior to protease exposure, the virus is blocked from interacting with cell surface receptors. The virus can be pretreated with protease, to release the lock from the capsid on the side with the first cleavage site, allowing the protein to adopt a more thermodynamically favorable conformation, such as a linear conformation, which may improve the ability of the second protease to cleave the second cleavage site, thereby unlocking the virus. Similarly, a single enzymatic cleavage site can be included, such that the virus can be pretreated with the corresponding protease which will release an inserted protein from the capsid on that end of the protein while the protein remains tethered to the capsid at the other end. This can allow the inserted protein to adopt a more thermodynamically favorable conformation, such as a linear conformation, which can enhance binding affinity and/or activity of the inserted protein or its target. FIGS. 3D and 3E similarly depict the peptide lock with two enzymatic cleavage sites, one for protease and one for MMPs. FIG. 3D shows an embodiment where the protease cleaves a first enzymatic cleavage motif, linearizing the inserted peptide, allowing the second enzyme, e.g. a MMP to cleave the remaining enzymatic cleavage motif to unlock the virus. FIG. 3E shows a similar embodiment, where the cleavages leave behind certain amino acids that were inserted on the surface of the capsid.

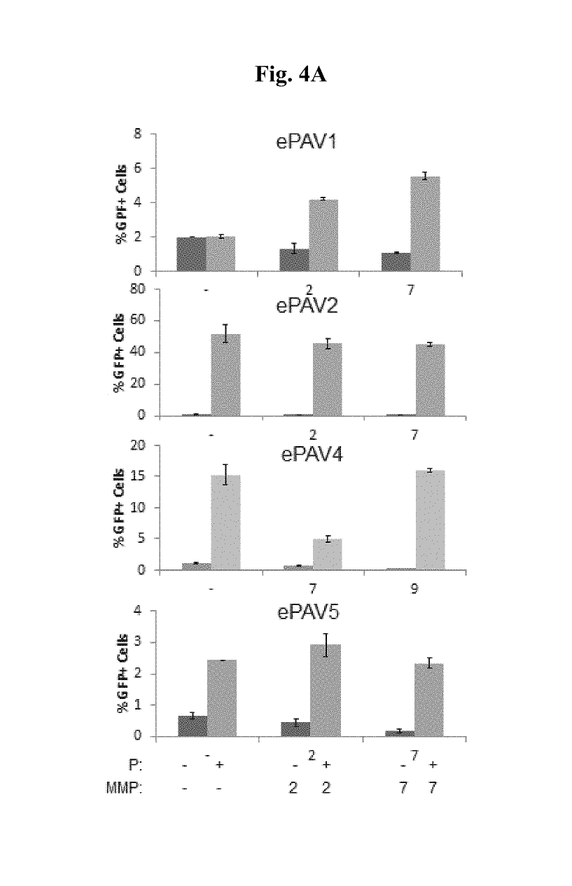

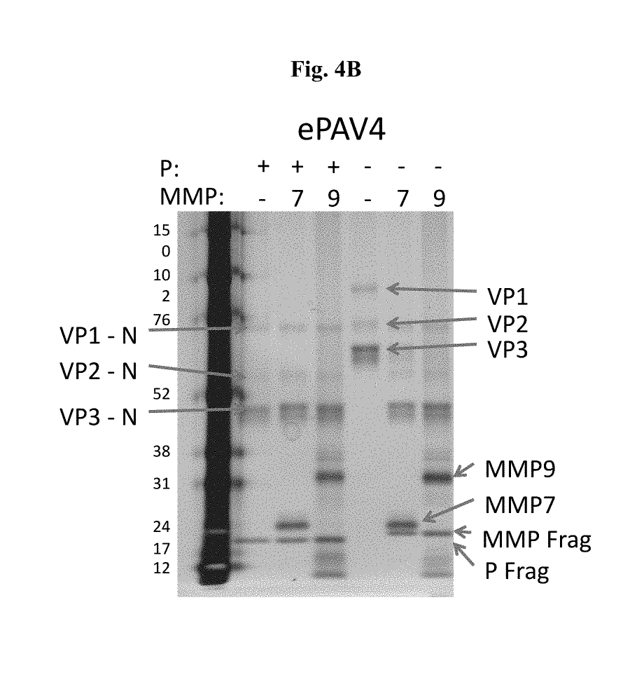

FIG. 4A shows the % GFP⁺ cells (indicative of transduction activity) after infection with AAV viruses having a peptide lock with two different enzymatic cleavage sites, one cleavable by a protease and one cleavable by a MMP, with or without pre-treatment with protease and with or without MMP-2 or MMP-7. The results show improved activity with pre-treatment using the protease indicating that the MMP is more efficiently able to cleave the second enzymatic cleavage site. FIG. 4B shows a silver stain of a gel containing virus ePAV4, which has two enzymatic cleavage sites, one for protease and one for MMPs, with or without pre-treatment with protease and with or without treatment with MMP-7 or MMP-9. The gel shows that intact virus is observed when the virus was treated with no proteases. N-terminal fragments were observed following treatment with any protease (indicated by “N”). Two different-size C-terminal fragments are observed that correspond to whether the MMP cleavage motif (“MMP Frag”) or the protease cleavage motif (“P Frag”) was cleaved.

Certain nucleotide sequences of MMP-2 can be found at SEQ ID NOs: 127-132, for MMP-7 at SEQ ID NO 133, and for MMP-9 at SEQ ID NOs: 134-135.

In certain embodiments, the virus and/or amino acid molecule can include or the nucleic acid molecule can encode an enzymatic cleavage motif adjacent to the optogenetic binding partner wherein the enzymatic cleavage motif does not inactivate other biologically active motifs on the surface of the virus. In some embodiments, the virus and/or amino acid can include or the nucleic acid molecule can encode an enzymatic cleavage motif adjacent to the optogenetic protein, portion thereof or variant thereof, wherein the enzymatic cleavage motif does not inactivate other biologically active motifs on the surface of the virus. By locating the enzymatic cleavage motif adjacent to the optogenetic binding partner or optogenetic protein, this allows for cleavage of the enzymatic cleavage motif by a protease, such as an endopeptidase or matrix metalloprotease (MMP). In some embodiments, the endopeptidase is enterokinase of SEQ ID NO: 76 (nucleotide sequence at SEQ ID NO: 75). Suitable proteases can include, by way of example but not limitation, trypsin, chymotrypsin, elastase, themolysin, pepsin, glutamyl endopeptidase, TEV protease, MMP-2, MMP-7 or MMP-9. In certain embodiments, the enzymatic cleavage motif can comprise the amino acid sequence of SEQ ID NO: 17 (PLGLAR), SEQ ID NO: 2 (IPESLRAG), SEQ ID NO: 1 (IPVSLRSG) SEQ ID NO: 18 (VPMSMRGG), or SEQ ID NO: 19 (Glu-Asn-Leu-Tyr-Phe-Gln/Gly). In some embodiments, the enzymatic cleavage motif is DDDDK (SEQ ID NO: 3) which is cleavable by enterokinase of SEQ ID NO: 76 (nucleotide sequence at SEQ ID NO: 75). In some embodiments, the enzymatic cleavage motif is Glu-Asn-Leu-Tyr-Phe-Gln-Gly (SEQ ID NO: 176) which is cleavable by TEV protease.

By permitting cleavage of at least one site adjacent to the optogenetic binding partner or optogenetic protein, the optogenetic binding partner or optogenetic protein can become detached from the capsid protein on that end of the optogenetic binding partner or optogenetic protein, improving the interaction the optogenetic binding partner with an optogenetic protein and vice versa. In some instances, the enzymatic cleavage can permit the linearization of the optogenetic binding partner or optogenetic protein and can enhance the interaction of the optogenetic protein or optogenetic binding partner, respectively. In certain instances, the enzymatic cleavage motif can act as a lock which limits the activity of the optogenetic binding partner or optogenetic protein until treatment with the corresponding protease which can cleave the enzymatic cleavage motif. The protease can be present in vivo, such as a MMP which is tissue specific or disease-specific and “activates” the optogenetic binding partner or optogenetic protein upon delivery to the tissue or diseased tissue. The protease can also be applied as a pre-treatment to “activate” the optogenetic binding partner or optogenetic protein for subsequent delivery to a target cell. The target cell can be in a human subject.

More broadly, the present disclosure also provides for a method for linearizing or conformationally unconstraining a surface peptide that is attached to a capsid protein of virus, such as AAV, more preferably AAV2. In such embodiments, a virus includes a capsid protein and one or more peptides genetically encoded into the capsid so as to be at least partially exposed to the surface of the capsid and the one or more peptides are adjacent to at least one enzymatically cleavable motif which can be cleaved by an enzyme, such as a protease. In some embodiments, the one or more peptides can block biologically active domains on the virus capsid surface. In some embodiments, the one or more peptides are adjacent to a first portion of the capsid protein to the N-terminal end of each peptide and a second portion of the capsid protein adjacent to the C-terminal end of each peptide. In other embodiments, the one or more peptides can be inserted adjacent to the N-terminus or C-terminus of the capsid protein. In some instances, the one or peptides and enzymatic cleavage motif can be inserted in the sequence of a capsid protein of the virus, for example, VP1 (SEQ ID NO: 50), VP2 (SEQ ID NO: 52) and/or VP3 (SEQ ID NO: 54) of AAV2. The site of insertion can vary based on the desired surface accessibility of the enzymatic cleavage motif. Various lengths of linkers flanking the one or more peptides may be employed to meet the desired surface accessibility as well as to provide more of less flexibility for the one or more peptides.

Because the one or more peptides are attached to the capsid at both the N-terminal end and C-terminal end of the peptides, in certain embodiments, they are constrained from adopting certain conformations, even though they are exposed on the capsid surface. Through cleavage of the enzymatic cleavage motif, the one or more peptides are freed and can adopt more thermodynamically favorable conformations, such as a linear conformation. For example, treatment with enterokinase of a virus with the one or more peptides exposed on the capsid surface with a DDDDK (SEQ ID NO: 3) enzymatic cleavage motif will liberate the end of the one or more peptides nearest to the enzymatic cleavage motif from the capsid, allowing for increased freedom for the one or more peptides to adopt favorable conformations while still tethered to the capsid surface on the other end. If removal of the enterokinase is desired, this can be achieved using various methods, such as treatment with trypsin-inhibitor agarose beads.

In some embodiments, the virus and/or amino acid molecule can include or the nucleic acid molecule can further encode a second enzymatic cleavage motif which is cleavable by a second enzyme that is different from the first enzyme which can cleave the first enzymatic cleavage motif. This second enzymatic cleavage motif can be located adjacent to the one or more peptides at the opposite end of the one more peptides from the first enzymatic cleavage motif. Once the first enzymatic cleavage motif is cleaved and the one or more peptides can adopt a more natural, tertiary structure, the second enzymatic cleavage motif can become more accessible to the second enzyme, such as a MMP. Thus, a virus with one or more peptides on the capsid surface can be pre-treated to cleave the first enzymatic cleavage motif, e.g. DDDDK (SEQ ID NO: 3), using the first enzyme, e.g. enterokinase, which can then be optionally removed, e.g. using trypsin-inhibitor agarose beads, to yield a virus with the one or more peptides tethered to the capsid surface with a second enzymatic cleavage motif, e.g. cleavable by a MMP, present which can be subsequently cleaved, e.g. in vivo.

In some embodiments, the peptide is a “biologically active domain” or “biologically active motif” which can alter the function of the virus, for example, by inhibiting cell binding. A “biologically active domain” (also known as a “biologically active motif”) is understood to be a peptide, protein or portion thereof that is capable of interacting with a biological molecule, generating a biological effect, or providing a detectable signal. Examples of such peptides or proteins include, but are not limited to a protease-cleavable peptide, a cell targeting peptide, a stealth-immune invading peptide, a protease, a post-translational modification enzyme, a light-activable protein, a fluorescent protein and a therapeutic protein. In some embodiments, the peptide can block a “biologically active domain” on the surface of the virus, such as HSPG to inhibit cell binding. In some instances, it is desirable that the peptide does not inactivate other biologically active motifs on the surface of the virus.

In some embodiments, a method is provided which includes the steps of providing an adeno-associated virus as described in the present disclosure which has an enzymatic cleavage motif incorporated and a protein exposed on the surface of the capsid protein adjacent to the enzymatic cleavage site and treating the virus with an enzyme to cleave the enzymatic cleavage motif. The virus, protein, enzymatic cleavage motif and enzyme can be as described in the present disclosure.

Viral Synthesis Methods

In some embodiments, a method is provided for synthesizing a virus. The method can comprise the steps of: (a) obtaining a nucleic acid molecule encoding a virus or portion thereof as described above; (b) transfecting the nucleic acid molecule into a cell to permit expression of the amino acid sequence(s) encoded by the nucleic acid molecule and assembly of the virus, wherein the virus comprises a capsid protein and an inserted protein; (c) isolating the virus from the cell. In certain embodiments, the virus can also include an enzymatic cleavage motif adjacent to the inserted protein and the method further comprises a step of treating the virus with an enzyme that recognizes and cleaves the enzymatic cleavage domain. In some embodiments, the method can further comprise removing the enzyme. In some embodiments, the method can further include administering the virus to a target cell. The capsid protein, inserted protein, enzymatic cleavage motif, enzyme and methods for removing the enzyme as well as administration of the virus to a target cell are further described throughout the present disclosure.

### EXAMPLES

### Example 1: Generation of a Modified-AAV2 with the Optogenetic Binding Partner PIF6

Recombinant adeno-associated virus serotype 2 (AAV2) was prepared as described by Xiao et al. (J. Virology, 2002). HEK293T cells were transfected using polyethylenimine with pXX2 (SEQ ID NO: 70, rep gene at SEQ ID NO: 71, cap gene at SEQ ID NO: 72) which carries the AAV2 rep and cap genes, the adenovirus helper plasmid pXX6-80 (SEQ ID NO: 69), and pAV-GFP (SEQ ID NO: 78) encoding green fluorescent protein (GFP) driven by a cytomegalovirus (CMV) promoter.

To generate AAV2 viruses with the 100 amino acid (aa) N-terminus of PIF6, which is capable of binding to activated PhyB holoprotein and which does not affect the cellular binding ability of the AAV2 virus through the heparin sulfate proteoglycan (HSPG) receptor, fused to the N-terminus of the VP2 capsid subunit (VNP-2-PIF6 (SEQ ID NO: 45, amino acid sequence at SEQ ID NO: 46), also referred to as VNP-PIF6), pXX2 (SEQ ID NO: 70) in the transfection mixture was substituted with plasmids pVP2A-PIF6 (SEQ ID NO: 73) and pRC_RR_VP1/3 (SEQ ID NO: 77) in a 4:1 ratio following the trans-complementing AAV capsid production scheme of Warrington, et al. (J. Virology, 2004) which allows for separate expression of VP1, VP2 and/or VP3. pVP2A-PIF6 contains the N-terminal 100 amino acids of PIF6 inserted at the N-terminus of VP2, flanked by MluI and FagI restriction sites and was generated using pVP2A as a starting construct. pVP2A has mutated VP1 and VP3 start codons to prevent their expression, and the weak VP2 start codon (CTG) is altered to a strong start (ATG).

A similar approach was followed for VNP-1,2-PIF6 except that pVP2A was replaced with pVP1,2A (SEQ ID NO: 74) to achieve fusion of the N-terminal 100 amino acids of PIF6 to both VP1 and VP2 capsid subunits—at the N-terminus of VP2 and at M138 of VP1 which does not affect the cellular binding ability of AAV2 through the HSPG receptor (SEQ ID NO: 114 for pVP-1,2A-PIF6)—and pRC_RR_VP1/3 was replaced with pRC_RR_VP3 to supplement wild-type VP3 (a VP3 construct supplying VP3 is pVP3 which can be found below under Additional Sequence Information), which is generally intolerant to insertions without compromising virus assembly and function.

HEK293T cells were harvested 48 hours after transfection and virus was separated from cell debris by iodixanol gradient ultracentrifugation. Virus was purified by heparin affinity chromatography with HiTrap Heparin HP columns (GE), and for electron microscopy and cellular studies virus was then dialyzed into Dulbecco's phosphate buffered solution (DPBS) with Ca²⁺ and Mg²⁺. Virus titers were measured via quantitative polymerase chain reaction (qPCR) with SYBR green (Life Technologies) reporter dye and using primers against the CMV promoter in the GFP transgene cassette,

| (SEQ ID NO: 21) | |
|---|---|
| FWD: | TCACGGGGATTTCCAAGTCTC |
| (SEQ ID NO: 22) | |
| REV: | AATGGGGCGGAGTTGTTACGAC |

The resulting titers from 3 independent virus batches for each virus with corresponding standard error measurements (SEM) are shown in Table 2 below:

TABLE 2
Viral Titers of wtAAV2, VNP-2-PIF6 and VNP-1,2-PIF6 Viruses

| Virus | Titer (genomes/mL) |
|---|---|
| wtAAV2 | 5.9 × 10¹¹ +/− 9.1 × 10¹⁰ |
| VNP-2-PIF6 | 4.7 × 10¹¹ +/− 1.4 × 10¹¹ |
| VNP-1,2-PIF6 | 4.1 × 10¹⁰ +/− 1.5 × 10¹⁰ |

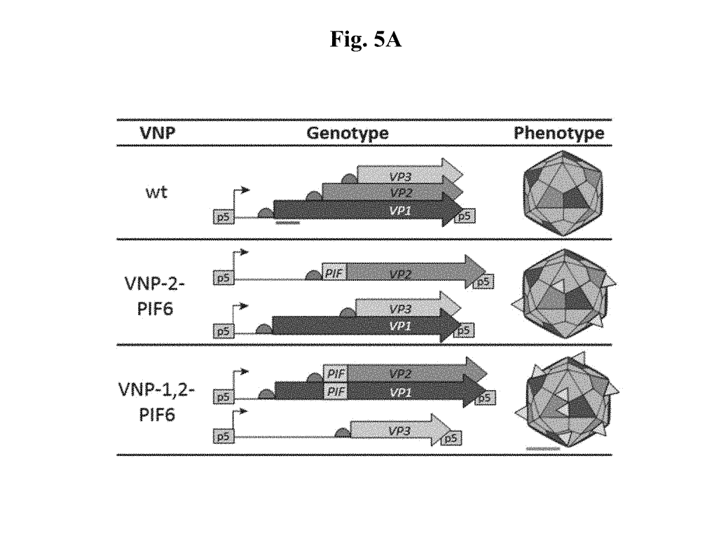

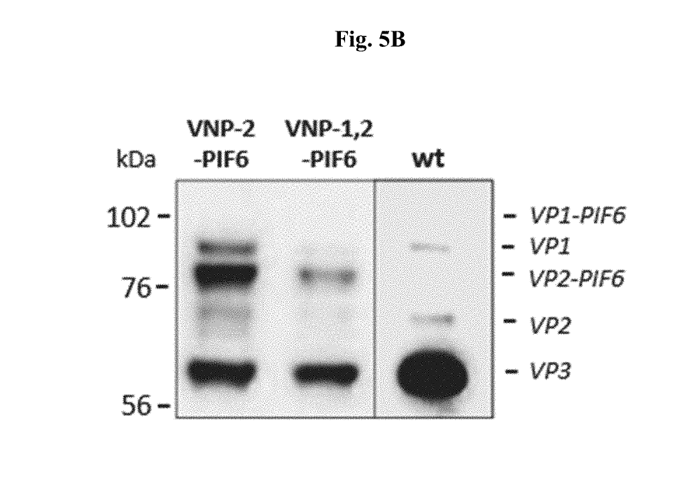

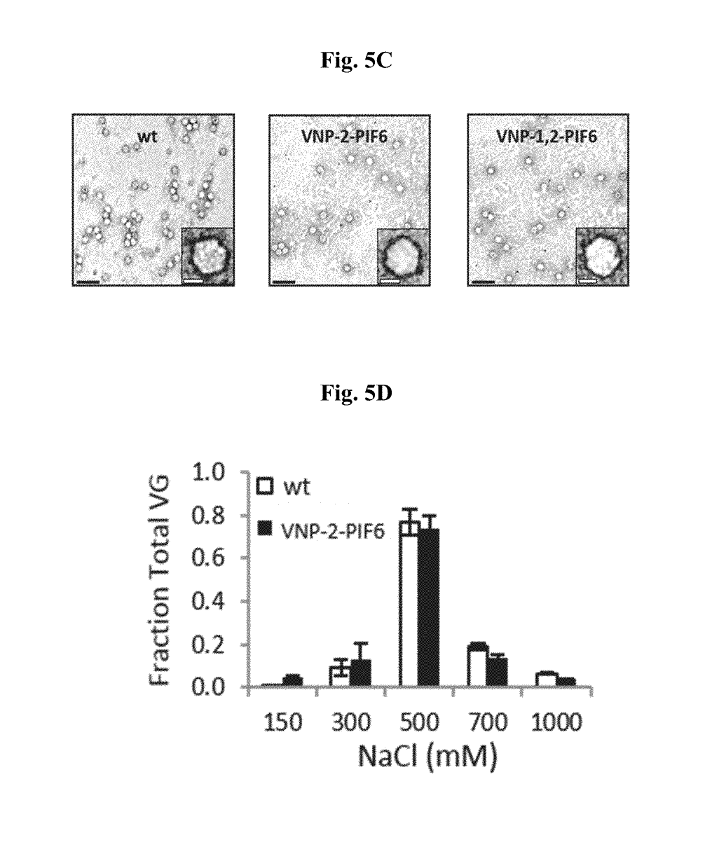

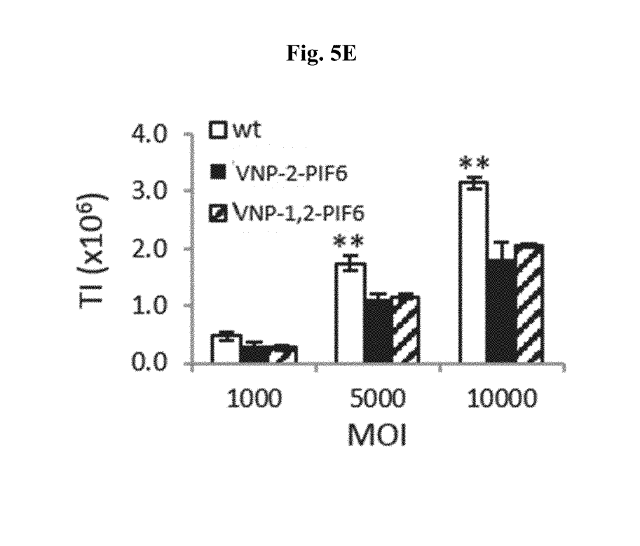

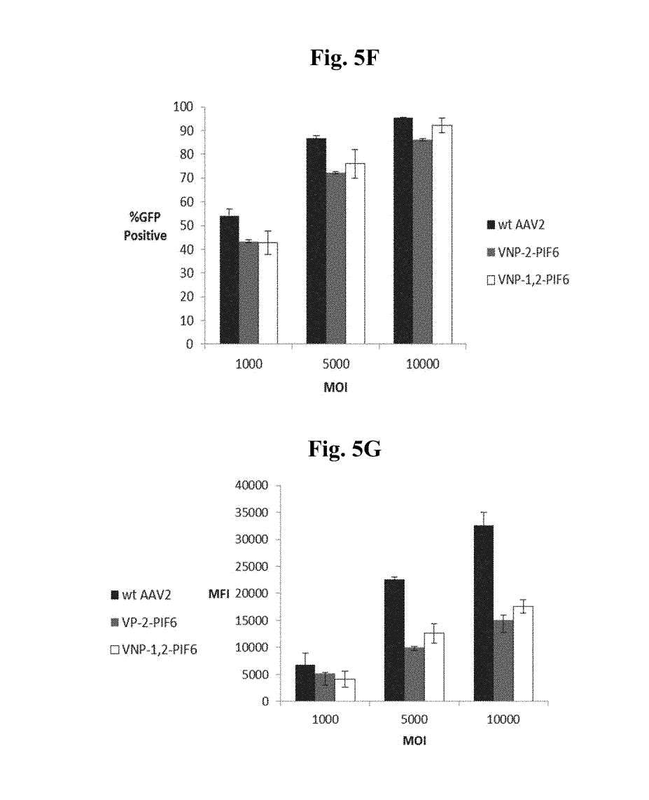

FIG. 5A shows the construct designs for producing wild-type (wt), VNP-2-PIF6, and VNP-1,2-PIF6 AAV2 viruses. Semi-circles indicate ribosomal binding site and all constructs were flanked by p5 promoter/enhancer elements. VP1, VP2 and VP3 are color-coded by shading as shown and PIF6 is shown in as triangles on the surface of the viral phenotype for VNP-2-PIF6 and VNP-1,2-PIF6.

The viruses, designated wt for wild-type, VNP-2-PIF6 (or VNP-PI6) for AAV2 with PIF6 fused to the N-terminus of VP2, and VNP-1,2-PIF6 for AAV2 with PIF6 fused to the N-terminus of VP2 and at inserted M138 of VP1, were resolved on 4-12% Bis-Tris NuPAGE gels (Life Technologies) and transferred to nitrocellulose (GE Healthcare) at 40V for 90 minutes. Blocking was performed in 5% skim milk in phosphate buffered saline (PBS) with 0.1% Tween-20 (PBS-T) for 1 hour while rocking. Blots were rinsed 3 times and rocked for 20 minutes in PBS-T. Primary antibodies were applied to blots overnight at 4° C. in PBS with 3% bovine serum albumin (BSA) (3% BSA-PBS) at the following dilutions: BI (monoclonal mouse anti-VP1, 2, 3 antibody from American Research Products) diluted 1:50. After washing, goat anti-mouse (Jackson ImmunoResearch) peroxidase-conjugated secondary antibody was applied at a 1:2,000 dilution in 5% skim milk in PBS-T for 1 hour. Blots were then washed 3 times for 15 minutes with PBS-T while rocking. Imaging was performed on a Fujifilm LAS 4000 with Lumi-Light western blotting substrate (Roche).

The resulting blots are shown in FIG. 5B. The results demonstrate the presence of VP2-PIF6 (the 100 N-terminal amino acids of PIF6 fused to the N-terminus of VP2) in both VNP-2-PIF6 and VNP-1,2-PIF6. VP1-PIF6 was not detected. Western blot densitometry indicated that VNP-2-PIF6 exhibits a VP stoichiometry of 1:7:22 for VP1:VP2:VP3 suggesting around 14 copies of VP2-PIF6 per capsid.

Virus samples purified into DPBS were applied to charged 300 mesh carbon grids (Ted Pella, Redding, Calif.) for 5 minutes. Samples were washed and negative stained with 0.75% uranyl formate to stain viral capsids and imaged on a JEOL 2010 transmission electron microscope operating at 120 kV (JEOL, Tokyo, Japan). The electron micrographs are shown in FIG. 5C. As demonstrated, the viruses show no distinct morphological differences with both VNP-2-PIF6 and VNP-1,2-PIF6 resembling wild-type morphology.

Viruses were also tested for heparin binding. Virus in iodixanol were incubated for 15 minutes with heparin-agarose beads (Sigma) resuspended in Tris-HCl with 150 mM NaCl. Sample were centrifuged at 6,000×g for 5 minutes to pellet beads and the supernatant was collected. Beads with bound virus were then resuspended sequentially in Tris-HCl containing NaCl at 300, 500, 700 and 1000 mM, with the supernatant collected at each step. Viral genomes were collected in each fraction and were quantified by qPCR for 2 independent experiments in duplicate, the results shown in FIG. 5D. As demonstrated, VNP-2-PIF6 has a similar heparin binding profile to wild-type AAV2 which indicates no change in native receptor binding due to PIF6 insertion.

Transduction efficiencies for each virus were also tested. HEK293T cells were seeded at 1×10⁵ cells/well on poly-L-lysine-coated 48-well plates approximately 30 hours before virus was added to cells (at 1,000, 5,000 or 10,000 MOI) in serum-free media. Fresh media containing serum was added 4 hours post-transduction and cells were harvested at 48 hours for flow cytometry analysis of mean fluorescence intensities and percentage of GFP-expressing cells on a BD FACSCanto II. Viral transduction ability was assessed by quantifying the transduction index (TI=% GFP+cells×geometric mean fluorescence intensity), a linear indicator of virus activity. The transduction index for each virus is shown in FIG. 5E from 2 independent experiments conducted in triplicate for wtAAV2 and VNP-2-PIF6 and 2 independent experiments conducted in duplicate for VNP-1,2-PIF6. As demonstrated, wtAAV2 shows a higher basal level of transduction than the two mutants with PIF6 insertions. The percentage of cells expressing GFP and mean fluorescence intensities from 4 independent experiments conducted in duplicate are shown in FIGS. 5F and 5G. The reduction in TI of VNP-2-PIF6 can be advantageous because it provides a wider dynamic range for tuning transduction.

### Example 2: Binding of Mutant AAV2 with PIF6 to PhvB

For in vitro binding studies, PhyB917 from Arabidopsis Thaliana was codon optimized for expression in Dictyostelium discoideum (Dd). A C-terminal hexahistidine tag (SEQ ID NO: 23) was added via iterative golden gate ligation with BsaI sticky ends using the following primers:

FWD:
(SEQ ID NO: 24)
GCATTAGGTCTCTAATGGTATCTGGTGTTGGTGGTTC
REV-1:
(SEQ ID NO: 25)
ATGATGATGATGATGATGACCACCACCACCTACTGCAAGAGCTTGTTGTA
ATTCTGG
REV-2:
(SEQ ID NO: 26)
GCTAATGGTCTCTTTTAATGATGATGAATGATGATGACCACC

PhyB917-His₆ was cloned by golden gate litigation into expression vector pDM323 downstream of the constitutive promoter P_{act15}. PhyB917-His₆ (SEQ ID NO: 42, nucleotide sequence at SEQ ID NO: 41) was mutated via site-directed mutagenesis (QuikChange, Agilent Genomics) to obtain PhyB917(Y276H)-His₆ (SEQ ID NO: 123, nucleotide sequence at SEQ ID NO: 122; non-His tagged sequence at SEQ ID NO: 40 with corresponding nucleotide sequence at SEQ ID NO: 39). PhyB651-His₆ which lacks a portion of the PHY domain, a motif conserved in all phytochromes that plays a role in the spectroscopic and photochemical properties of the protein, was cloned into a pET28a/Tev/His6 vector (SEQ ID NO: 177) was obtained from Dr. M. Rosen (UT Southwestern, TX). For studies in cells, pKM216 (SEQ ID NO: 117), pKM017 (SEQ ID NO: 118), and pKM018 (SEQ ID NO: 119) encoding PhyB908 (SEQ ID NO: 36, nucleotide sequence at SEQ ID NO: 35), PhyB908-NLS (SEQ ID NO: 38, nucleotide sequence at SEQ ID NO: 37), and PhyB650-NLS (SEQ ID NO: 34, nucleotide sequence at SEQ ID NO: 33, non-NLS sequence at SEQ ID NO: 32 with corresponding nucleotide sequence at SEQ ID NO: 31), respectively, were obtained from Dr. W. Weber (University of Freiburg, Germany).

Dd strain AX4 was transformed with plasmids pEG03 (SEQ ID NO: 124) and pEG04 (SEQ ID NO: 125) encoding PhyB917-His₆ (SEQ ID NO: 42) and PhyB917(Y276H)-His₆ (SEQ ID NO: 123), respectively, by standard electroporation protocol. Single transformants were harvested from Klebsiella aerogenes-SM agar plates after 3 days and transferred to liquid HL5 media. Axenic cultures (50 mL, 22° C., 180 rpm) were grown to a density of 1×10⁷ cells/mL and harvested by centrifugation (500×g, 5 minutes).

PhyB651-His₆ was transformed into E. coli strain BL21(DE3) by electroporation and plated onto LB agar containing kanamycin (30 μg/mL) and chloramphenicol (34 μg/mL). Bacteria were then cultured in liquid LB containing kanamycin and chloramphenicol at 18° C. Cells were induced with 0.5 mM IPTG at OD₆₀₀=0.04-0.06 for at least 24 hours before being harvested by centrifugation (4,000×g, 10 minutes).

Following harvesting by centrifugation, all PhyB variants were separated from cell lysate by repeated freeze/thaw cycles to lyse cells, and centrifugation at 3,000×g for 10 minutes in the presence of Protease Inhibitor Cocktail (Sigma). Purification from supernatant was performed by nickel affinity chromatography (His Spintrap, GE Healthcare) according to manufacturer's protocol.

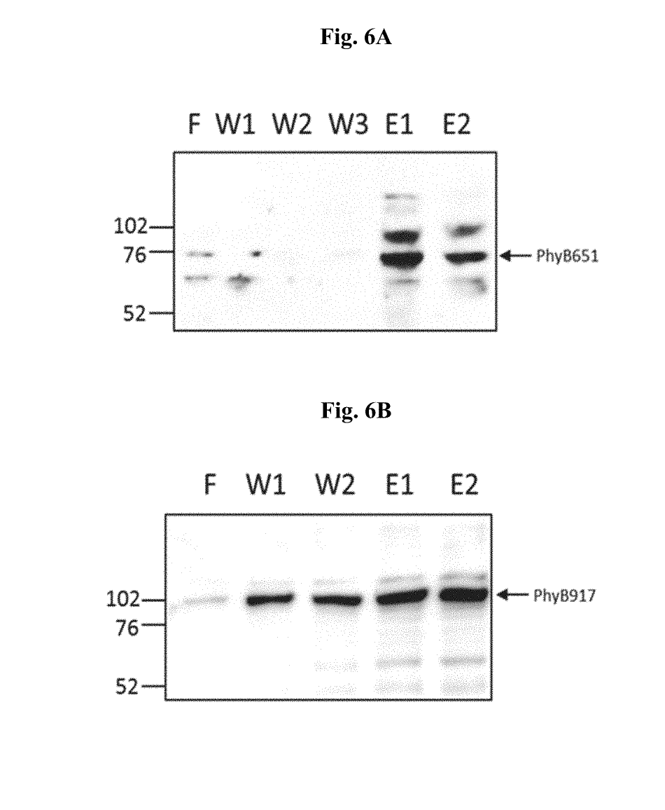

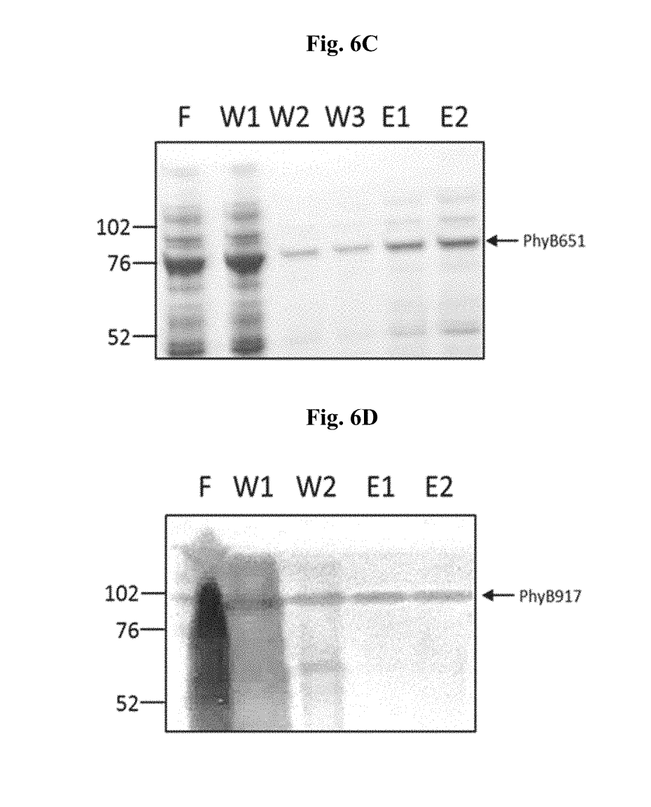

PhyB651-His₆ and PhyB917-His₆ (SEQ ID NO: 42) after nickel purification were analyzed via Western blot as described in Example 1, using anti-His₆ (“His₆” disclosed as SEQ ID NO: 23) (monoclonal mouse antibody from American Research Products) diluted 1:50 instead of B 1. The resulting Western blots are shown in FIGS. 6A-6B. Corresponding coomassie stained gels showing purified Ni²⁺ fractions are shown in FIG. 6C-6D. As demonstrated, highly purified PhyB651-His₆ (76 kDa) and PhyB917-His₆ (102 kDa) (SEQ ID NO: 42) were obtained.

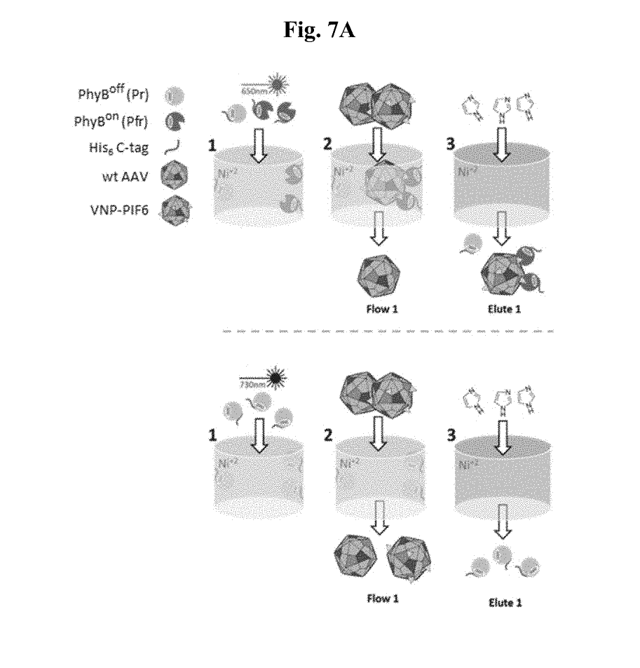

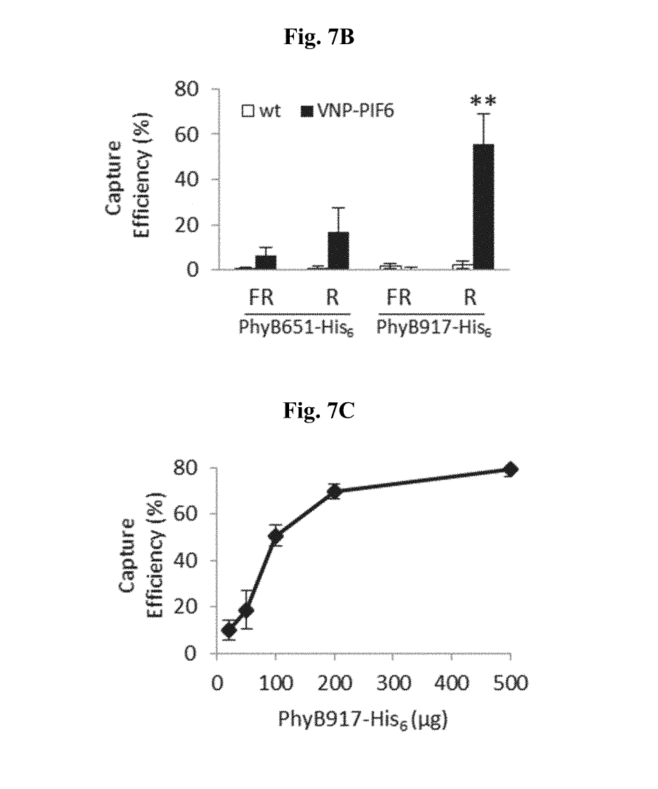

Binding of wtAAV2 and VNP-2-PIF6 to the expressed PhyB-His₆ was assessed using in vitro binding assays as depicted in FIG. 7A. As shown in FIG. 7A His₆-tagged PhyB proteins (“His₆” disclosed as SEQ ID NO: 23) can be immobilized on nickel columns 1, virus can then be flowed through the column with the wtAAV flowing through and VNP-2-PIF6 binding to the PhyB proteins 2 followed by elution of the bound VNP-2-PIF6 and PhyB protein using imidazole 3.

PhyB651-His₆ and PhyB-917-His₆ (SEQ ID NO: 42) were diluted in binding buffer (20 mM NaPO₄, 500 mM NaCl, 20 mM imidazole, pH 7.4) and incubated for 30 minutes with phycocyanobilin (PCB) at a final concentration of 5 μM under green light (500 nm) to prevent chromophore bleaching, and then exposed to either 650 nm (red) or 730 nm (far-red) light. PhyB651-His₆ and PhyB-917-His₆ (SEQ ID NO: 42) were each bound to separate Ni²⁺ columns (His Spintrap, GE Healthcare) via centrifugation at 100×g for 30 seconds, and wtAAV or VNP-2-PIF6 diluted in binding buffer were added to the columns in the presence of 650 nm or 730 nm light. After a 2 minute incubation, columns were washed twice and bound viruses eluted with elution buffer (20 mM NaPO₄, 500 mM NaC, 500 mM imidazole, pH 7.4) as per the manufacturer's protocol. Viral genomes present in each fraction were quantified by qPCR. Capture efficiency was determined as viral titer in the eluted fractions divided by the total amount of virus added to the column. The capture efficiencies for PhyB651-His₆ and PhyB-917-His₆ (SEQ ID NO: 42) from 3 independent experiments in duplicate are shown in FIG. 7B. As demonstrated in FIG. 7B, neither wtAAV2 nor VNP-2-PIF6 binds to PhyB917-His₆ (SEQ ID NO: 42) or PhyB651-His₆ in any appreciable amount under far-red (FR) light while VNP-2-PIF6 binds PhyB917-His₆ (SEQ ID NO: 42) 24-fold better than wtAAV2 under red (R) light, a statistically significant difference. VNP-2-PIF6 also binds PhyB651-His₆ 17-fold more compared to wild-type virus under red light. In addition, PhyB917 has a broader dynamic range, capturing 3-fold more VNP-2-PIF6 than PhyB651-His₆ under red light and almost 10-fold less under far red light. Experiments were also performed using different amounts of PhyB protein, specifically PhyB917-His₆ (SEQ ID NO: 42) for column loading. The results of 2 independent experiments in duplicate are shown in FIG. 7C and demonstrate that the amount of VNP-2-PIF6 captured is a function of the presence of PhyB and not nonspecific binding to the column, with 80% capture efficiency achieved at 500 μg of PhyB917-His₆ (SEQ ID NO: 42) under red light (activating) conditions (approximately 4×10⁹ genome-packaging viruses captured out of 5×10⁹).

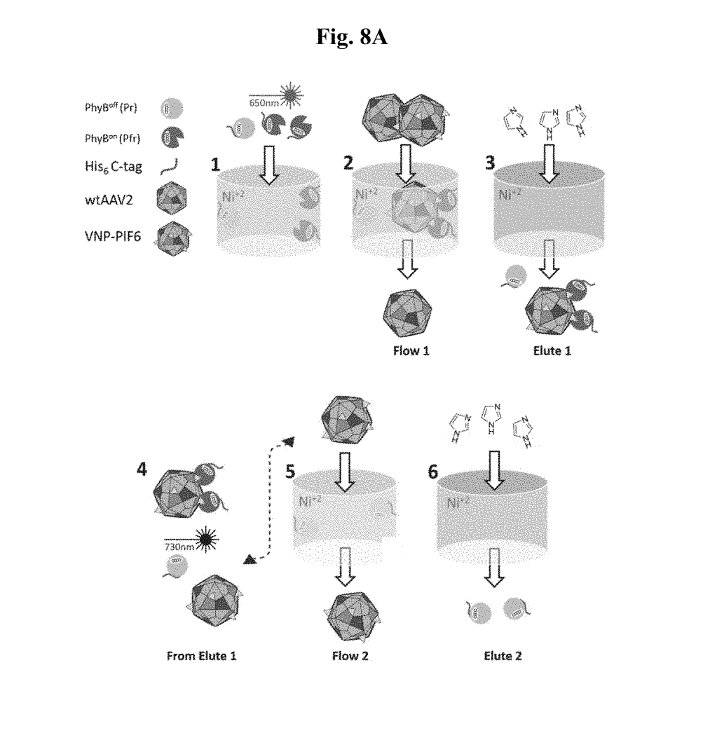

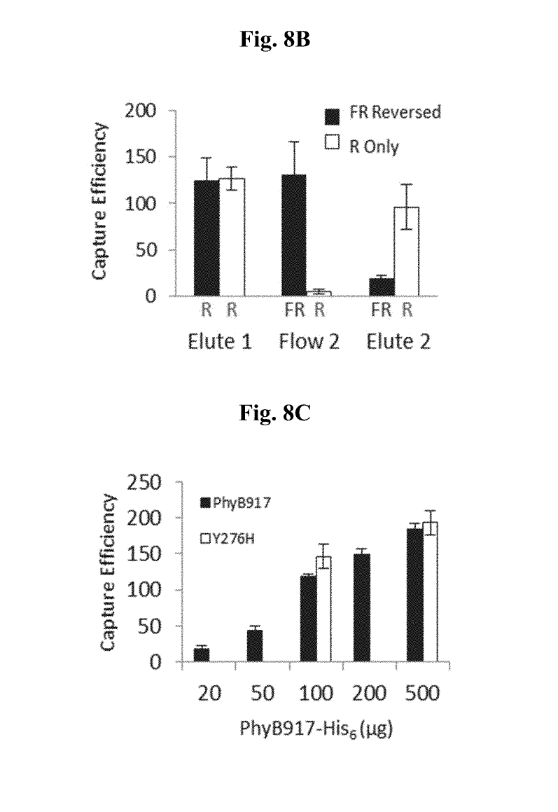

The reversibility of the binding of VNP-2-PIF6 to PhyB917-His₆ (SEQ ID NO: 42) under far red (FR) light conditions was also assessed as shown in FIG. 8A. Ni²⁺ column elution fractions containing activated PhyB917-His₆ bound to VNP-2-PIF6 were diluted to 20 mM imidazole and exposed to FR light for 20 minutes 4. The FR-treated samples were then applied to a new Ni²⁺ column, and new flow 5 and elution 6 fractions were collected. Samples were analyzed by qPCR as above and the results of 3 independent experiments in duplicate at 100 μg PhyB917-His₆ (SEQ ID NO: 42) are shown in FIG. 8B. After inactivation with FR light, the majority of VNP-2-PIF6 was detected in flow through (Flow 2), and not in the following elution fraction (Elute 2) which indicates that VNP-2-PIF6 binding to PhyB917-His₆ (SEQ ID NO: 42) is reversible with FR light exposure. Control samples which were not FR-treated resulted in a majority of viruses still bound to the column and eluting in Elute 2.

To confirm that the light-induced dissociation and binding is the result of photoactivation of the phytochrome, the PhyB917(Y276H)-His₆ (SEQ ID NO: 123) mutant, which is constitutively active was tested alongside PhyB917-His₆ (SEQ ID NO: 42) as described above using varying amounts of each phytochrome. The capture efficiencies for each were measured in 2 independent experiments in duplicate and the results are shown in FIG. 8C. As demonstrated, at the two amounts tested for the Y276H mutant-100 μg and 500 μg—the capture efficiency was comparable to PhyB917-His₆ (SEQ ID NO: 42), indicating that the binding is the result of the phytochrome and not a nonspecific effect.

These results demonstrate reversible binding of AAV2 expressing the first 100 amino acids of PIF6 on the capsid surface to soluble PhyB in vitro that is light-inducible, being activated under red light conditions and deactivated under far red light conditions.

### Example 3: In Vivo Nuclear Localization Studies

To test whether VNP-2-PIF6 can be used to facilitate increased nuclear localization over wtAAV using its light-inducible binding to PhyB, a confocal microscopy study was performed.

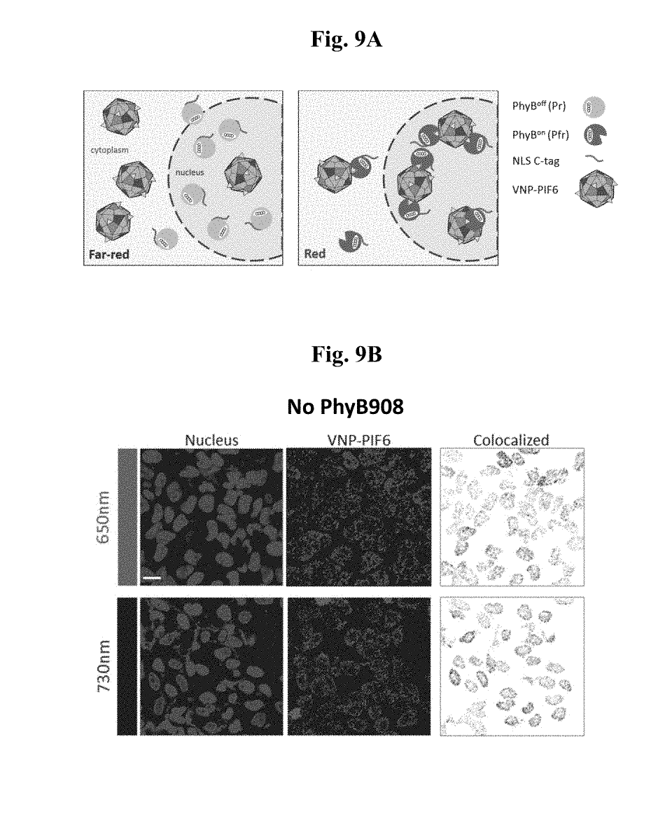

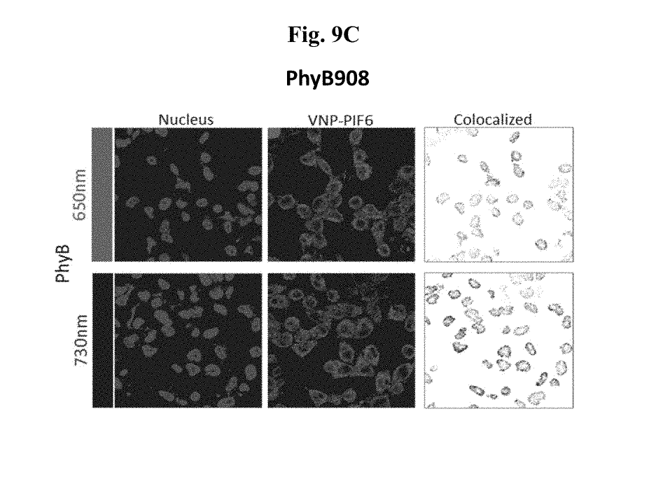

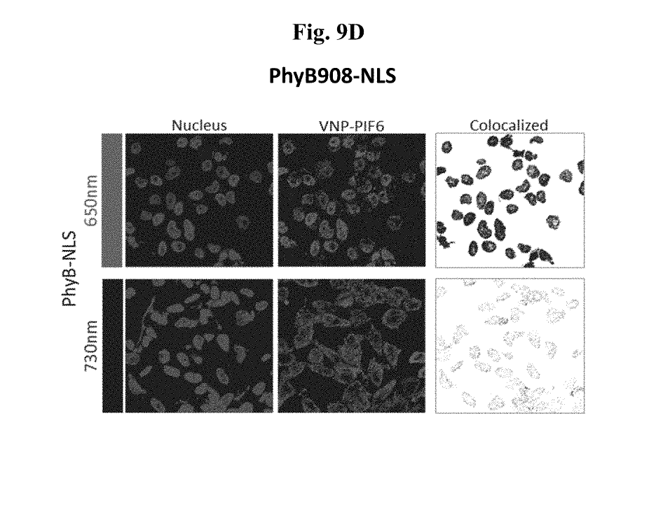

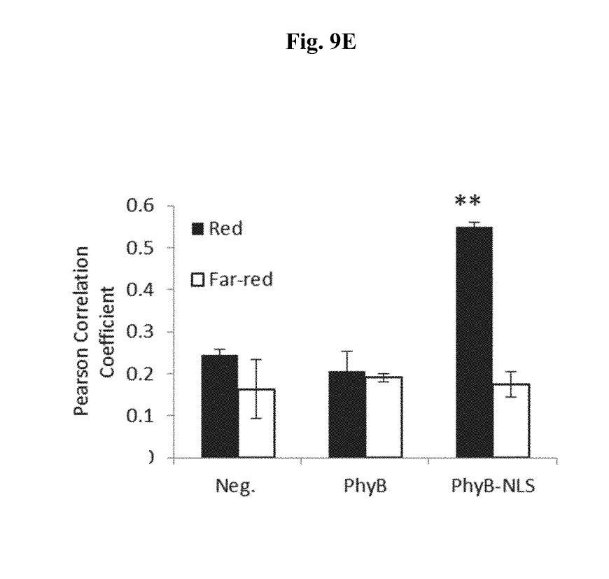

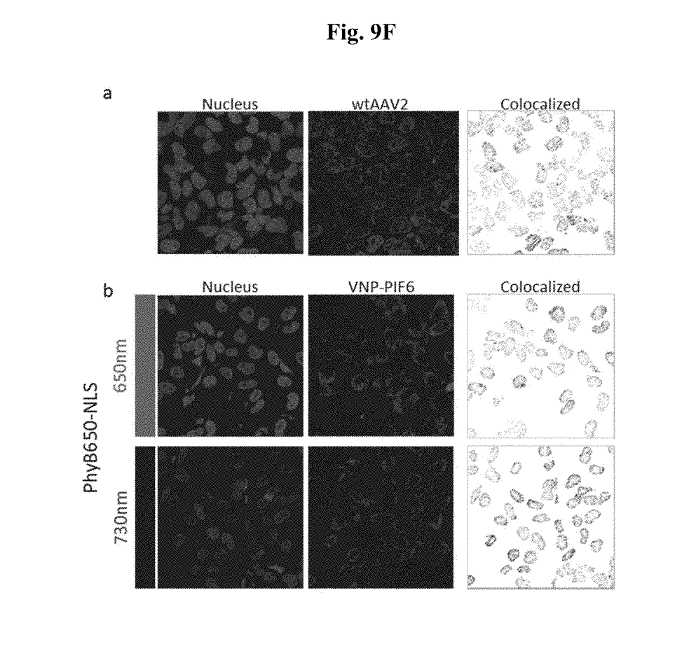

FIG. 9A shows the expected mechanism for light-activable gene delivery using VNP-PIF6 in the presence of PhyB with a NLS fusion under deactivating (Far Red, left panel) or ambient light and activating (Red, right panel) light. Under activating conditions, the PhyB-NLS adopts a conformation capable of binding PIF6 and binds the VNP-PIF6 which enhances nuclear uptake of the virus through the NLS, while under deactivating conditions and/or ambient conditions, the PIF6 dissociates from and does not bind the PhyB-NLS, resulting in basal levels of nuclear uptake.

HeLa cells were seeded onto poly-L-lysine-coated glass coverslips in a 24-well tissue culture plate at a density of 8×10⁴ cells per well in Dulbecco's modified Eagle's medium (DMEM) supplemented with 10% fetal bovine serum and 1% penicillin-streptomycin. After 4 hours, cells were transfected with polyethylenimine (PEI)-DNA complexes (N/P=20) encoding PhyB908 (a variant which is analogous the PhyB917) with or without a C-terminal NLS fusion (SEQ ID NOs: 38 and 36, respectively, corresponding nucleotide sequences at SEQ ID NOs: 37 and 35, respectively). A negative control group of wells were not transfected. 24 hours later, under green light (500 nm), PCB at a final concentration of 15 μM, and virus (VNP-2-PIF6 or wtAAV2, purified into DPBS with Mg²⁺ and Ca²⁺) at an MOI of 5,000 were applied to cells in serum-free media. Cells were then incubated for 4 hours at 37° C., 5% CO₂ under R or FR light.

Immunofluorescence analysis was performed. Cells were washed twice with PBS and fixed with 4% paraformaldehyde for 30 minutes. Next, cells were permeabilized with warm 0.1% Triton for 10 minutes, washed twice with PBS, and blocked in 3% BSA-PBS for 30 minutes with rocking. Primary antibody A20 (monoclonal mouse anti-AAV2 intact capsid from American Research Products) diluted 1:125, was added and cells were incubated overnight at 4° C. with gentle agitation. After washing three times with PBS and 5 minute incubations, secondary fluorescent probe donkey anti-mouse IgG-CFL (Santa Cruz Biotechnology) was added at 1:250 dilution and cells were rocked in the dark for 2 hours. Cells were washed 3 times and stained with Hoescht nuclear stain (0.1 μg/mL) for 15 minutes with rocking in the dark. After washing twice more in PBS, cells were incubated with 4% paraformaldehyde for 15 minutes and mounted onto glass slides in 3 μL of Fluoromount-G (SouthernBiotech). Samples were imaged on a Zeiss LSM 710 confocal microscope and the resulting images, processed using ImageJ. are shown in FIG. 9B through FIG. 9D. The results demonstrate that under red light, nuclear accumulation of VNP-2-PIF6 is dramatically increased in cells expressing PhyB908-NLS (SEQ ID NO: 38) compared to control cells, cells expressing PhyB908 without a NLS (SEQ ID NO: 36) and those exposed to far red light. In the control cells, cells expressing PhyB908 without a NLS and those exposed to far red light, the viruses are mostly in the cytoplasm or aggregated in the perinuclear space.

Image of the colocalization of the VNP-2-PIF6 signal and the nucleus signal was performed. Images were processed using Zen 2010 software (Carl Zeiss MicroImaging) and ImageJ. Measurements were determined over two fields of view for each sample, with an average of 40 cells per field of view. tM (Nuc) is the proportion of all nuclear signal overlapped by virus signal. tM (Virus) is the proportion of all virus signal overlapped by nuclear signal. Nuclear and AAV signals were uniformly thresholded using the ImageJ JACoP plugin. Qualitative colocalization images were processed using ImageJ. The Pearson correlation coefficients, from 2 independent experiments, and thresholded Manders' coefficients reveal a statistically significant higher co-localization between VNP-PIF6 and the nucleus only in cells expressing PhyB908-NLS and exposed to activating R light as shown in FIG. 9E and Table 3.

TABLE 3
Virus-nucleus colocalizaton statistics

| PhyB type | Virus | Light | tM (nuc) | tM (virus) |
|---|---|---|---|---|
| — | wtAAV2 | — | 0.13 | 0.52 |
| — | VNP-2-PIF6 | FR | 0.08 | 0.45 |
| — | VNP-2-PIF6 | R | 0.07 | 0.39 |
| PhyB650-NLS | VNP-2-PIF6 | FR | 0.12 | 0.47 |
| PhyB650-NLS | VNP-2-PIF6 | R | 0.10 | 0.25 |
| PhyB908 | VNP-2-PIF6 | FR | 0.13 | 0.41 |
| PhyB908 | VNP-2-PIF6 | R | 0.08 | 0.33 |
| PhyB908-NLS | VNP-2-PIF6 | FR | 0.06 | 0.40 |
| PhyB908-NLS | VNP-2-PIF6 | R | 0.45** | 0.64** |

**= Differences between co-localization of VNP-2-PIF6 with PhyB908-NLS and R light, and all other conditions are statistically significant (p < 0.05) by unpaired Student's t-test.

A similar experiment was performed using PhyB650 (a variant which is analogous to PhyB651) (SEQ ID NO: 32) with or without a C-terminal NLS fusion, however, PhyB650-NLS did not affect the intracellular distribution of VNP-PIF6, potentially due to its lower binding affinity for PIF6 and partial ablation of the PhyB PAS domain which has been shown to result in weak or a complete lack of PhyB binding to PIF6. In addition, it is possible that the C-terminal NLS tag was not recognized by cellular importins due to obstruction or other steric effects. FIG. 9F shows the colocalization of wtAAV2 and of VNP-PIF6 in cells constitutively expressing PhyB650-NLS under red light and far red light conditions.

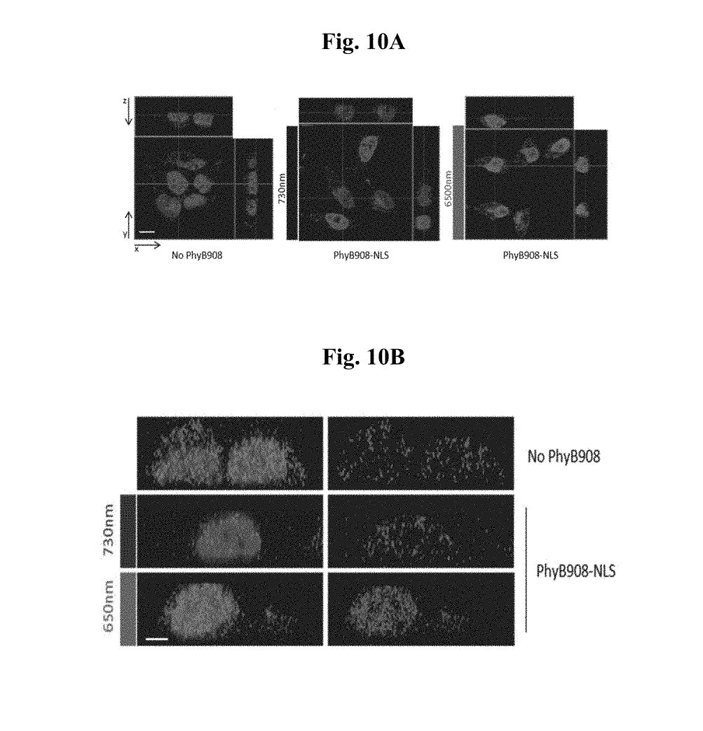

To confirm that the nuclear localization of VNP-2-PIF6 is not a 2-dimensional artifact, three-dimensional Z-stacks were obtained with confocal microscopy. Visualizing cell nuclei slice through the x-, y- and z-axis as shown in FIG. 10A, and closer inspection of y-axis individual channel slices as shown in FIG. 10B confirmed higher VNP-2-PIF6 signal inside the nucleus.

In combination, these data suggest that VNP-2-PIF6 selectively binds to activated (under red light) PhyB908-NLS under physiological conditions, leading to more effective nuclear translocation of the virus as compared to the wtAAV2.

### Example 4: Tuning of Gene Delivery by Ratiometric Control of Red Far Red Light

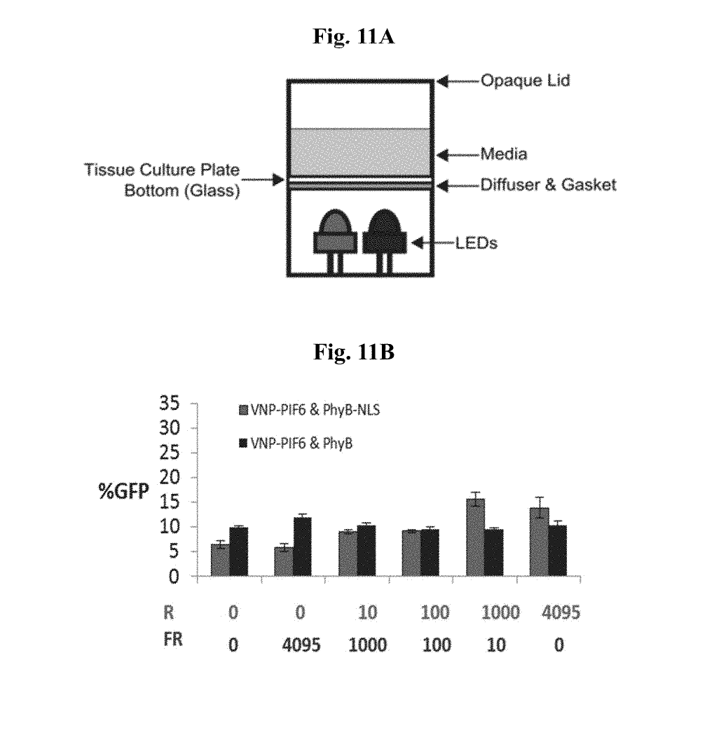

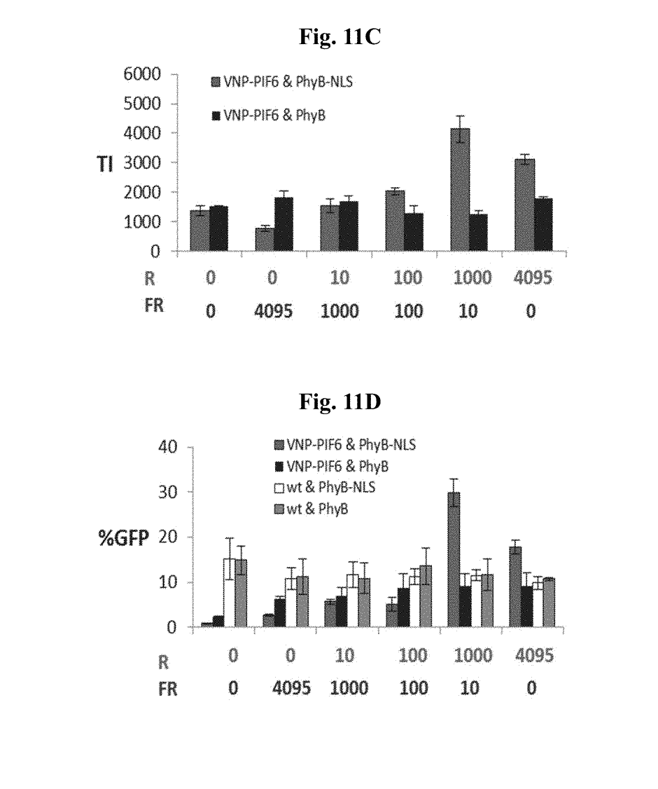

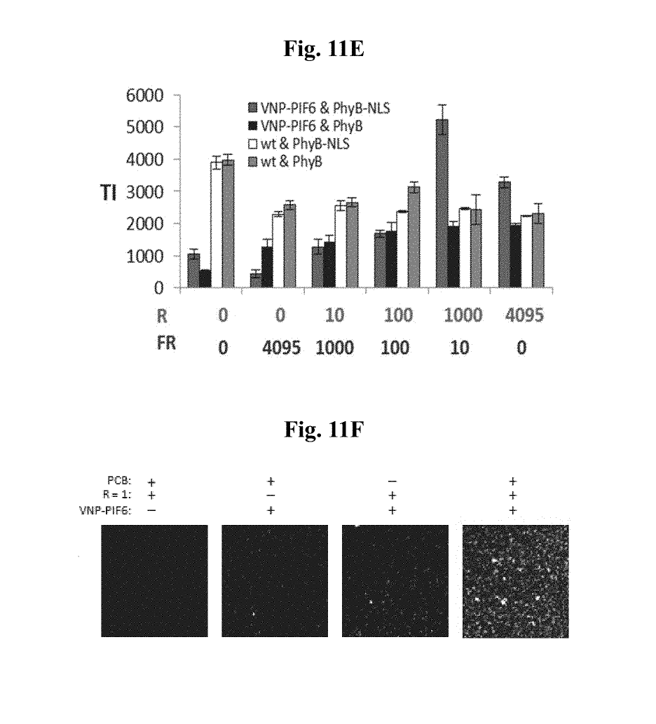

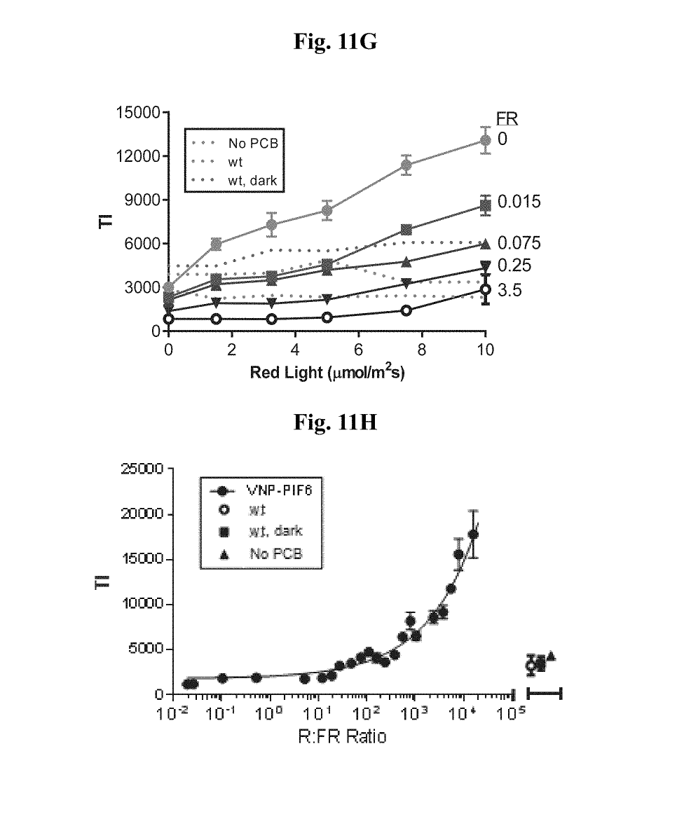

Modulating the R:FR light ratio can tune the efficiency of gene delivery. A custom LED-tissue culture plate apparatus as shown in FIG. 11A that shields each individual well from outside light was used. An Arduino Uno microcontroller was used to program a 6×4 array of optically isolated LEDs (LEDtronics, #L200CWRGB2K-4A-IL; Marubeni: L735-5AU) which can expose cells to 630 nm and 735 nm light simultaneously through the bottom of a 24-well black, glass-bottom tissue culture plate (Greiner bio-one, #662892). LED intensity was quantified and converted from raw Arduino units by placing a fiber optic photodetector probe (StellarNet Inc., photodetector #EPP2000 UVN-SR-25 LT-16, probe #F600-UV-VIS-SR) directly into tissue culture wells and measuring light flux, in units of μmol/m²s, for a range of intensities for R/FR light. The glass bottom of each well of the tissue culture plate was coated with poly-L-lysine and HeLa cells were seeded at a density of 1×10⁵ cells per well in DMEM supplemented with 10% fetal bovine serum and 1% penicillin-streptomycin. After 24 hours, cells were transfected with PEI-DNA (pKM017 (SEQ ID NO: 118) and pKM216 (SEQ ID NO: 117)) complexes encoding PhyB908 with or without a C-terminal NLS fusion. 24 hours later, under green light, PCB at a final concentration of 15 μM and virus (VNP-2-PIF6 or wtAAV2) at an MOI of 2,000 were applied in DMEM supplemented with 10% serum and incubated at 37° C., 5% CO₂. The LEDs were programmed to shine FR light for 5 minutes before switching to experiment-dependent intensities of R and/or FR light. Cells were harvested and prepared for flow cytometry on a BD FACSCanto II after 24 or 48 hours. The % of cells positive for GFP and the transduction index (TI), from 2 independent experiments, for the cells 24 hours post-transduction for varying ratios of R:FR light are shown in FIGS. 11B and 11C. The % of cells positive for GFP and the transduction for the cells 48 hours post-transduction for varying ratios of R:FR light, from 2 independent experiments, are shown in FIGS. 11D and 11E. As demonstrated, PhyB908 without a NLS has no effect on gene delivery as compared to wtAAV2 (FIGS. 11D and 11E). However, PhyB908-NLS increased gene delivery as compared to wtAAV2 as the ratio of R:FR light increased and decreased gene delivery as the ratio of R:FR light decreased (FIGS. 11B-11E). Similar results are seen in FIGS. 11F-8G. FIG. 11F depicts fluorescence micrographs of GFP expression in the HeLa cells constitutively expressing PhyB908-NLS and treated with or without VNP-2-PIF6, PCB, and red light. As demonstrated, PCB and red light in combination with VNP-2-PIF6 result in a significant increase in GFP expression, indicating an increase in transduction. FIG. 11G shows the discrete transfer functions for transduction of VNP-2-PIF6 at red light flux between 0 and 10 μM/m²s with co-delivery of far red light as well as samples with no PCB, wtAAV2 instead of VNP-2-PIF6 with light delivery and wtAAV2 in the dark. The results show increasing transduction with VNP-2-PIF6 as the ratio of red light increases. FIG. 11H shows a dose-response curve for VNP-2-PIF6 based on the ratio of R:FR light with the response being measured as transduction index. This curve clearly demonstrates that the gene delivery efficiency of VNP-2-PIF6 increases dramatically as the R:FR light ratio increases, exponentially when plotted on a logarithmic scale. The dose-response curve can be fit as TI=Ax^{B}+C, where A=285, B=0.41, C=1800 and x is the R:FR light ratio with a r² value of 0.95.

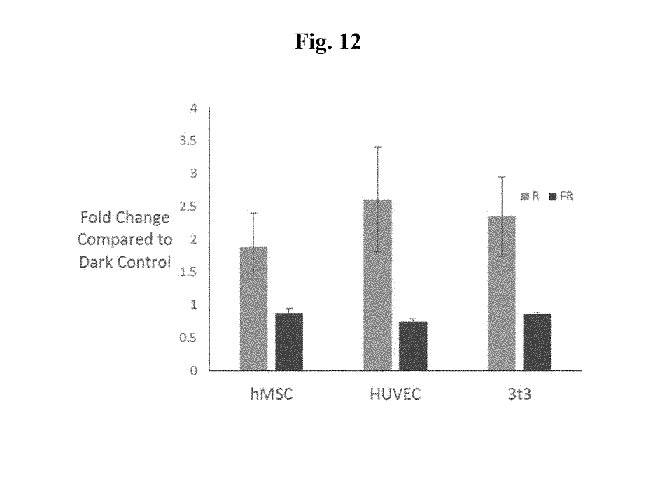

Thus, ratiometric control of the R:FR ratio of light can provide a method to tune transduction to increase or decrease gene delivery by increasing red light or far red light, respectively. The maximum level of 17,796 for transduction index was achieved at a R:FR ratio of 15,950 and R:FR ratios above about 250 allow VNP-2-PIF6 to more effectively transduce cells than wtAAV2. Further, the greater nuclear entry demonstrated correlates with increased transduction efficiency. In addition, the light-activable viral gene delivery platform can work in other cell types, including those for use in tissue engineering application such as human mesenchymal stem cells (hMSC), human umbilical vein endothelial cells (HUVEC), and 3T3 fibroblasts as show in FIG. 12 which shows about a 2-fold increase in transduction as compared to a dark control where the cells were treated as described in the foregoing example with either red light at 10.67 μM/m²s or far red light at 3.61 μM/m²s for 48 hours. The TI values achieved were 167,399 for hMSC, 106,866 for HUVEC and 10,524 for 3T3. Thus, even in a difficult to transduce cell line, 3T3, the light-activable system improved transduction.

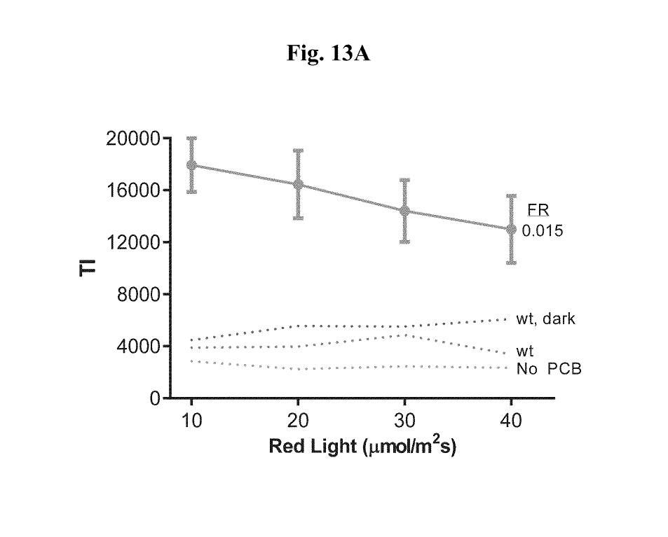

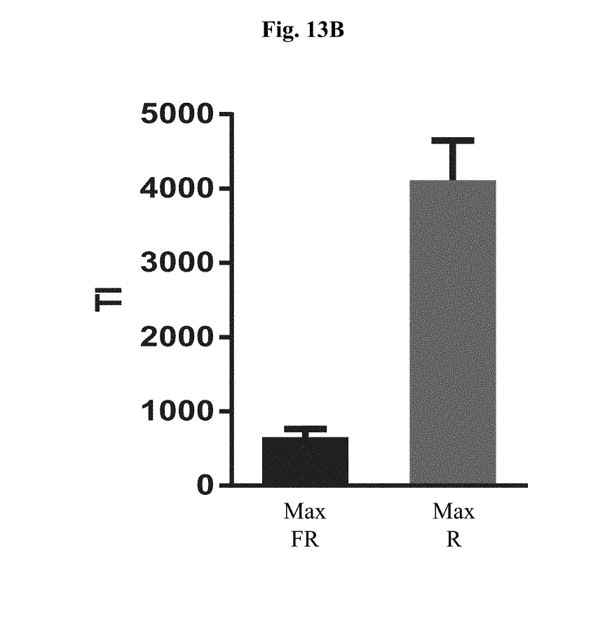

As shown in FIG. 13A, above a R:FR ratio of 16,000 the transduction index decreased monotonically. FIG. 13B shows the maximum transduction index for maximum far red and maximum red lights only. Thus, there may be a useful range of R:FR ratios that may be useful to increase the transduction index as compared to that for wtAAV2 depending on the optogenetic binding partner and protein used, the cell type, the growth conditions and other properties.

### Example 5: Spatial Control of Viral Gene Delivery Using R/FR Light

VNP-2-PIF6 can also provide for spatial control of gene delivery which may be an important parameter for achieving therapeutic outcomes. Photomask experiments were conducted following a published protocol for space-resolved gene expression. HeLa cells were cultured in a glass-bottom, poly-L-lysine-coated 24-well plate (Greiner bio-one, #662892) with opaque walls and ceilings. Photomasks were laser-etched into black nitrile sheets using a Universal X-660 laser cutter platform and placed under the wells. The photomask sheet also functioned as a gasket sealing the 24-well plate directly above the R/FR LEDs. HeLa cells were seeded at a density of 1×10⁵ cells per well in DMEM supplemented with 10% fetal bovine serum and 1% penicillin-streptomycin. After 24 hours, cells were transfected with PEI-DNA (pKM017 (SEQ ID NO: 118)) complexes encoding PhyB908 with a C-terminal NLS fusion. 24 hours later, under green light, PCB at a final concentration of 15 μM and virus (VNP-2-PIF6) at an MOI of 1,000 were applied in DMEM supplemented with 10% serum and incubated at 37° C., 5% CO₂. The LEDs were programmed to shine FR light (2 μmol/m²s) for 30 minutes before switching to experiment-dependent intensities of R or R/FR light for 60 minutes. Cells remained in the dark for the remainder of 48 hours before being fixed with 4% paraformaldehyde in PBS and imaged on a Nikon A1 microscope. Images were taken at 20× magnification and a 12×12 square array of images were stitched together. Image signal and brightness were processed in ImageJ using the Threshold function.

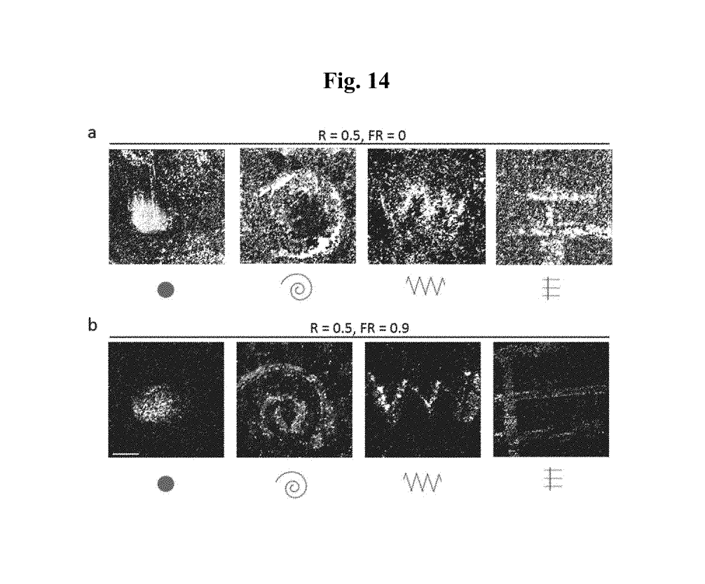

The resulting images are shown in FIG. 14 and demonstrate spatial control of improved transduction using the VNP-2-PIF6/PhyB908-NLS system. Using only red light (R=0.5 μmol/m²s; FR=0.0 μmol/m²s) resulted in high background noise in gene expression even at low flux. However, co-delivering far red light (R=0.5 μmol/m²s; FR=0.9 μmol/m²s) resulted in improved signal-to-noise and better resolved patterns.

These results demonstrate that the light-activable viral delivery system can be spatially controlled by limiting the location of exposure to activating light and that co-delivery of R/FR light can improve resolution. Because activation using light can also be controlled by when the light is introduced, the system provides temporal control in addition to spatial control over gene delivery efficiency which provides a powerful tool for not only improving, but controlling, gene delivery.

More broadly, the foregoing results demonstrate the utility of the optogenetic system for improving and controlling gene delivery using viral vectors using light.

### Example 6: Peptide Insertion and Use of Two Enzymatic Cleavage Motifs Adjacent to the Peptide

In an example, a peptide (AG-PLGLAR-G-DDDK-GA (SEQ ID NO: 27) or AG-DDDDK-G-PLGLAR-GA (SEQ ID NO: 28)) is inserted at amino acid position 586 in the AAV2 capsid which corresponds to position 586 in VP1, position 449 in VP2 and position 383 in VP3. PLGLAR (SEQ ID NO: 17) is a MMP-cleavable peptide motif and DDDDK (SEQ ID NO: 3) is an enterokinase-cleavable domain. Cleavage of the DDDDK (SEQ ID NO: 3) motif allows the PLGLAR (SEQ ID NO: 17) sequence to be displayed as a linearized MMP-cleavable substrate on the surface of the capsid. AG, G, and GA residues serve as linkers and cloning sites to facilitate peptide insertion using conventional molecular cloning methods. The MMP-cleavable motif can be changed from PLGLAR (SEQ ID NO: 17) to any suitable enzymatically cleavable motif or to a peptide of interest such that the peptide of interest is displayed on the surface of the virus but is less conformation constrained because it is only tethered to the virus at one end after pre-treatment with the enterokinase.

### Example 7: Peptide Insertion and Use of a Single Enzymatic Cleavage Motif Adjacent to the Peptide and Virus Generation

In an example, a peptide or protein can be genetically inserted via molecular cloning into the capsid protein sequence paired with a single enterokinase recognition motif either immediately before or after the peptide/protein sequence. The enzymatic cleavage motif, which can include DDDDK (SEQ ID NO: 3), and which is recognized and cleaved by enterokinase, is inserted adjancet to the desired peptide sequence. Plasmids encoding capsid proteins (altered or wild-type), transgene of interest, and helper proteins for virus assembly and packaging are transfected into HEK293T producer cells via polyethylenimine transfection. Cells are collected after 48 hours, lysed, and the virus is separated from cell debris via density gradient ultracentrifugation. Once viruses are made, they are digested (pre-treated) with enterokinase (SEQ ID NO: 76, nucleotide sequence at SEQ ID NO: 75) to linearize and/or conformationally unconstrain the peptide on the surface the capsid. Subsequent column purification with trypsin-inhibitor agarose beads binds the enterokinase to purify the virus sample for downstream use and analysis.

### Example 8: Enhancement of Transduction Efficiency by Use of and Enzymatic Cleavage Motif Adjacent to an Inserted Optogenetic Protein

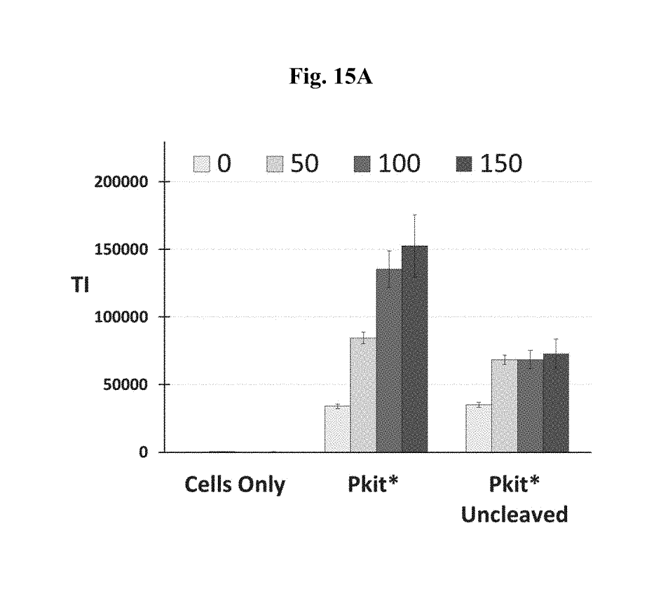

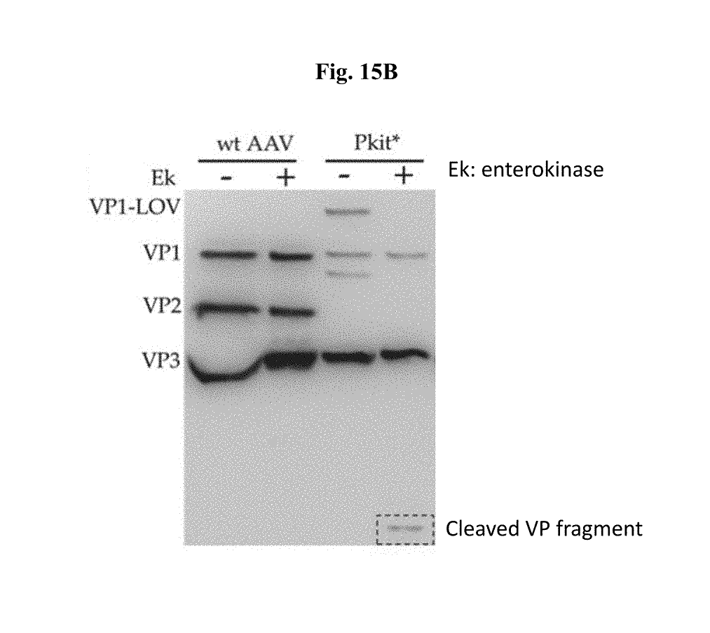

An AAV-based virus was prepared as described above using only VP1 and VP3 capsid proteins. The LOV domain from Avena sativa phototropin 1 protein with a C-terminal nuclear localization signal (TRPQRDCPTPTWQPQPRRKSW (SEQ ID NO: 6)) and an N-terminal nuclear export signal (MLALKLAGLDI (SEQ ID NO: 10)) was embedded in the capsid protein VP1 adjacent to an enzymatic cleavage motif (DDDDK (SEQ ID NO: 3)) (NES-LOV2-NLS encoded by nucleotide SEQ ID NO: 142, a similar nucleotide with LOV-NLS, lacing a nuclear export signal, can be found at SEQ ID NO: 141). Under blue light of about 450 nm, the LOV domain undergoes a conformational change which exposes the NLS which is otherwise occluded. As in the previously examples, GFP was used as a reporter for transduction. A control group of HeLa cells was not treated with virus. Two experimental groups were treated with the virus at an MOI of 1,000, the first group receiving the virus without pre-treatment with enterokinase, the second group receiving the virus after a 16-18 hour pre-treatment with enterokinase to cleave the enzymatic cleavage motif. Enterokinase (SEQ ID NO: 76, nucleotide sequence at SEQ ID NO: 75) treatment was performed in a 10 μL volume of CaCl₂) containing 1 μL of enterokinase. The control and experimental groups were exposed to blue light of about 470 nm for 12 hours, with four sub-groups within each group receiving 0, 50, 100 or 150 μmol/m²s of the blue light. After 48 hours post-transduction, the cells were harvested and analyzed for GFP expression as in the previous examples. The results are shown in FIG. 15A and demonstrate that pre-treatment with an enzyme to cleave the enzymatic cleavage motif results in improved transduction efficiency, especially at higher intensities of light. FIG. 15B shows a Western blot of the virus and of wild-type AAV2, with or without pre-treatment with enterokinase for 16 hours. The results demonstrate that wild-type virus is unaffected by enterokinase treatment and successful incorporation of the LOV domain in VP1 of the engineered virus.

The foregoing description of specific embodiments of the present disclosure has been presented for purpose of illustration and description. The exemplary embodiments were chosen and described in order to best explain the principles of the invention and its practical application, to thereby enable others skilled in the art to best utilize the invention and various embodiments with various modifications are suited to the particular use contemplated.

### Additional Sequence Information

pVP3, which can be used to provide VP3 alone in viral synthesis has a nucleotide sequence of:

| | |
|---|---|
| 1 | aattcccatc atcaataata taccttattt tggattgaag ccaatatgat aatgaggggg |
| 61 | tggagtttgt gacgtggcgc ggggcgtggg aacggggcgg gtgacgtagt agtctctaga |
| 121 | gtcctgtatt agaggtcacg tgagtgtttt gcgacatttt gcgacaccat gtggtcacgc |
| 181 | tgggtattta agcccgagtg agcacgcagg gtctccattt tgaagcggga ggtttgaacg |
| 241 | cgcagccacc acgccggggt tttacgagat tgtgattaag gtccccagcg accttgacgg |
| 301 | gcatctgccc ggcatttctg acagctttgt gaactgggtg gccgagaagg aatgggagtt |
| 361 | gccgccagat tctgacatgg atctgaatct gattgagcag gcacccctga ccgtggccga |
| 421 | gaagctgcag cgcgactttc tgacggaatg gcgccgtgtg agtaaggccc cggaggccct |
| 481 | tttctttgtg caatttgaga agggagagag ctacttccac atgcacgtgc tcgtggaaac |
| 541 | caccggggtg aaatccatgg ttttgggacg tttcctgagt cagattcgcg aaaaactgat |
| 601 | tcagagaatt taccgcggga tcgagccgac tttgccaaac tggttcgcgg tcacaaagac |
| 661 | cagaaatggc gccggaggcg ggaacaaggt ggtggatgag tgctacatcc ccaattactt |
| 721 | gctccccaaa acccagcctg agctccagtg ggcgtggact aatatggaac agtatttaag |
| 781 | cgcctgtttg aatctcacgg agcgtaaacg gttggtggcg cagcatctga cgcacgtgtc |
| 841 | gcagacgcag gagcagaaca aagagaatca gaatcccaat tctgatgcgc cggtgatcag |
| 901 | atcaaaaact tcagccaggt acatggagct ggtcgggtgg ctcgtggaca aggggattac |
| 961 | ctcggagaag cagtggatcc aggaggacca ggcctcatac atctccttca atgcggcctc |
| 1021 | caactcgcgg tcccaaatca aggctgcctt ggacaatgcg ggaaagatta tgagcctgac |
| 1081 | taaaaccgcc cccgactacc tggtgggcca gcagcccgtg gaggacattt ccagcaatcg |
| 1141 | gatttataaa attttggaac taaacgggta cgatccccaa tatgcggctt ccgtctttct |
| 1201 | gggatgggcc acgaaaaagt tcggcaagag gaacaccatc tggctgtttg ggcctgcaac |
| 1261 | taccgggaag accaacatcg cggaggccat agcccacact gtgcccttct acgggtgcgt |
| 1321 | aaactggacc aatgagaact ttcccttcaa cgactgtgtc gacaagatgg tgatctggtg |
| 1381 | ggaggagggg aagatgaccg ccaaggtcgt ggagtcggcc aaagccattc tcggaggaag |
| 1441 | caaggtgcgc gtggaccaga aatgcaagtc ctcggcccag atagacccga ctcccgtgat |
| 1501 | cgtcacctcc aacaccaaca tgtgcgccgt gattgacggg aactcaacga ccttcgaaca |
| 1561 | ccagcagccg ttgcaagacc ggatgttcaa atttgaactc acccgccgtc tggatcatga |
| 1621 | ctttgggaag gtcaccaagc aggaagtcaa agactttttc cggtgggcaa aggatcacgt |
| 1681 | ggttgaggtg gagcatgaat tctacgtcaa aaagggtgga gccaagaaaa gacccgcccc |
| 1741 | cagtgacgca gatataagtg agcccaaacg ggtgcgcgag tcagttgcgc agccatcgac |
| 1801 | gtcagacgcg gaagcttcga tcaactacgc agacaggtac caaaacaaat gttctcgtca |
| 1861 | cgtgggcatg aatctgatgc tgtttccctg cagacaatgc gagagaatga atcagaattc |
| 1921 | aaatatctgc ttcactcacg gacagaaaga ctgtttagag tgctttcccg tgtcagaatc |
| 1981 | tcaacccgtt tctgtcgtca aaaaggcgta tcagaaactg tgctacattc atcatatcat |
| 2041 | gggaaaggtg ccagacgctt gcactgcctg cgatctggtc aatgtggatt tggatgactg |
| 2101 | catctttgaa caataaatga tttaaatcag gtctggctgc cgatggttat cttccagatt |
| 2161 | ggctcgagga cactctctct gaaggaataa gacagtggtg gaagctcaaa cctggcccac |
| 2221 | caccaccaaa gcccgcagag cggcataagg acgacagcag gggtcttgtg cttcctgggt |
| 2281 | acaagtacct cggacccttc aacggactcg acaagggaga gccggtcaac gaggcagacg |
| 2341 | ccgcggccct cgagcacgac aaagcctacg accggcagct cgacagcgga gacaacccgt |
| 2401 | acctcaagta caaccacgcc gacgcggagt ttcaggagcg ccttaaagaa gatacgtctt |
| 2461 | ttgggggcaa cctcggacga gcagtcttcc aggcgaaaaa gagggttctt gaacctctgg |
| 2521 | gcctggttga ggaacctgtt aaggcggctc cgggaaaaaa gaggccggta gagcactctc |
| 2581 | ctgtggagcc agactcctcc tcgggaaccg gaaaggcggg ccagcagcct gcaagaaaaa |
| 2641 | gattgaattt tggtcagact ggagacgcag actcagtacc tgacccccag cctctcggac |
| 2701 | agccaccagc agccccctct ggtctgggaa ctaatacgat ggctacaggc agtggcgcac |
| 2761 | caatggcaga caataacgag ggcgccgacg gagtgggtaa ttcctcggga aattggcatt |
| 2821 | gcgattccac atggatgggc gacagagtca tcaccaccag cacccgaacc tgggccctgc |
| 2881 | ccacctacaa caaccacctc tacaaacaaa tttccagcca atcaggagcc tcgaacgaca |
| 2941 | atcactactt tggctacagc accccttggg ggtattttga cttcaacaga ttccactgcc |
| 3001 | acttttcacc acgtgactgg caaagactca tcaacaacaa ctggggattc cgacccaaga |
| 3061 | gactcaactt caagctcttt aacattcaag tcaaagaggt cacgcagaat gacggtacga |
| 3121 | cgacgattgc caataacctt accagcacgg ttcaggtgtt tactgactcg gagtaccagc |
| 3181 | tcccgtacgt cctcggctcg gcgcatcaag gatgcctccc gccgttccca gcagacgtct |
| 3241 | tcatggtgcc acagtatgga tacctcaccc tgaacaacgg gagtcaggca gtaggacgct |
| 3301 | cttcatttta ctgcctggag tactttcctt ctcagatgct gcgtaccgga aacaacttta |
| 3361 | ccttcagcta cacttttgag gacgttcctt tccacagcag ctacgctcac agccagagtc |
| 3421 | tggaccgtct catgaatcct ctcatcgacc agtacctgta ttacttgagc agaacaaaca |
| 3481 | ctccaagtgg aaccaccacg cagtcaaggc ttcagttttc tcaggccgga gcgagtgaca |
| 3541 | ttcgggacca gtctaggaac tggcttcctg gaccctgtta ccgccagcag cgagtatcaa |
| 3601 | agacatctgc ggataacaac aacagtgaat actcgtggac tggagctacc aagtaccacc |
| 3661 | tcaatggcag agactctctg gtgaatccgg gcccggccat ggcaagccac aaggacgatg |
| 3721 | aagaaaagtt ttttcctcag agcggggttc tcatctttgg gaagcaaggc tcagagaaaa |
| 3781 | caaatgtgga cattgaaaag gtcatgatta cagacgaaga ggaaatcagg acaaccaatc |
| 3841 | ccgtggctac ggagcagtat ggttctgtat ctaccaacct ccagagaggc aacagacaag |
| 3901 | cagctaccgc agatgtcaac acacaaggcg ttcttccagg catggtctgg caggacagag |
| 3961 | atgtgtacct tcaggggccc atctgggcaa agattccaca cacggacgga cattttcacc |
| 4021 | cctctcccct catgggtgga ttcggactta aacaccctcc tccacagatt ctcatcaaga |
| 4081 | acaccccggt acctgcgaat ccttcgacca ccttcagtgc ggcaaagttt gcttccttca |
| 4141 | tcacacagta ctccacggga caggtcagcg tggagatcga gtgggagctg cagaaggaaa |
| 4201 | acagcaaacg ctggaatccc gaaattcagt acacttccaa ctacaacaag tctgttaatg |
| 4261 | tggactttac tgtggacact aatggcgtgt attcagagcc tcgccccatt ggcaccagat |
| 4321 | acctgactcg taatctgtaa ttgcttgtta atcaataaac cgtttaattc gtttcagttg |
| 4381 | aactttggtc tctgcgtatt tctttcttat ctagtttcca tgctctagag tcctgtatta |
| 4441 | gaggtcacgt gagtgttttg cgacattttg cgacaccatg tggtcacgct gggtatttaa |
| 4501 | gcccgagtga gcacgcaggg tctccatttt gaagcgggag gtttgaacgc gcagccacca |
| 4561 | cggcggggtt ttacgagatt gtgattaagg tccccagcga ccttgacggg catctgcccg |
| 4621 | gcatttctga cagctttgtg aactgggtgg ccgagaagga atgggagttg ccgccagatt |
| 4681 | ctgacatgga tctgaatctg attgagcagg cacccctgac cgtggccgag aagctgcatc |
| 4741 | gctggcgtaa tagcgaagag gcccgcaccg atcgcccttc ccaacagttg cgcagcctga |
| 4801 | atggcgaatg gaattccaga cgattgagcg tcaaaatgta ggtatttcca tgagcgtttt |
| 4861 | tcctgttgca atggctggcg gtaatattgt tctggatatt accagcaagg ccgatagttt |
| 4921 | gagttcttct actcaggcaa gtgatgttat tactaatcaa agaagtattg cgacaacggt |
| 4981 | taatttgcgt gatggacaga ctcttttact cggtggcctc actgattata aaaacacttc |
| 5041 | tcaggattct ggcgtaccgt tcctgtctaa aatcccttta atcggcctcc tgtttagctc |
| 5101 | ccgctctgat tctaacgagg aaagcacgtt atacgtgctc gtcaaagcaa ccatagtacg |
| 5161 | cgccctgtag cggcgcatta agcgcggcgg gtgtggtggt tacgcgcagc gtgaccgcta |
| 5221 | cacttgccag cgccctagcg cccgctcctt tcgctttctt cccttccttt ctcgccacgt |
| 5281 | tcgccggctt tccccgtcaa gctctaaatc gggggctccc tttagggttc cgatttagtg |
| 5341 | ctttacggca cctcgacccc aaaaaacttg attagggtga tggttcacgt agtgggccat |
| 5401 | cgccctgata gacggttttt cgccctttga cgttggagtc cacgttcttt aatagtggac |
| 5461 | tcttgttcca aactggaaca acactcaacc ctatctcggt ctattctttt gatttataag |
| 5521 | ggattttgcc gatttcggcc tattggttaa aaaatgagct gatttaacaa aaatttaacg |
| 5581 | cgaattttaa caaaatatta acgtttacaa tttaaatatt tgcttataca atcttcctgt |
| 5641 | ttttggggct tttctgatta tcaaccgggg tacatatgat tgacatgcta gttttacgat |
| 5701 | taccgttcat cgattctctt gtttgctcca gactctcagg caatgacctg atagcctttg |
| 5761 | tagagacctc tcaaaaatag ctaccctctc cggcatgaat ttatcagcta gaacggttga |
| 5821 | atatcatatt gatggtgatt tgactgtctc cggcctttct cacccgtttg aatctttacc |
| 5881 | tacacattac tcaggcattg catttaaaat atatgagggt tctaaaaatt tttatccttg |
| 5941 | cgttgaaata aaggcttctc ccgcaaaagt attacagggt cataatgttt ttggtacaac |
| 6001 | cgatttagct ttatgctctg aggctttatt gcttaatttt gctaattctt tgccttgcct |
| 6061 | gtatgattta ttggatgttg gaattcctga tgcggtattt tctccttacg catctgtgcg |
| 6121 | gtatttcaca ccgcatatgg tgcactctca gtacaatctg ctctgatgcc gcatagttaa |
| 6181 | gccagccccg acacccgcca acacccgctg acgcgccctg acgggcttgt ctgctcccgg |
| 6241 | catccgctta cagacaagct gtgaccgtct ccgggagctg catgtgtcag aggttttcac |
| 6301 | cgtcatcacc gaaacgcgcg agacgaaagg gcctcgtgat acgcctattt ttataggtta |
| 6361 | atgtcatgat aataatggtt tcttagacgt caggtggcac ttttcgggga aatgtgcgcg |
| 6421 | gaacccctat ttgtttattt ttctaaatac attcaaatat gtatccgctc atgagacaat |
| 6481 | aaccctgata aatgcttcaa taatattgaa aaaggaagag tatgagtatt caacatttcc |
| 6541 | gtgtcgccct tattcccttt tttgcggcat tttgccttcc tgtttttgct cacccagaaa |
| 6601 | cgctggtgaa agtaaaagat gctgaagatc agttgggtgc acgagtgggt tacatcgaac |
| 6661 | tggatctcaa cagcggtaag atccttgaga gttttcgccc cgaagaacgt tttccaatga |
| 6721 | tgagcacttt taaagttctg ctatgtggcg cggtattatc ccgtattgac gccgggcaag |
| 6781 | agcaactcgg tcgccgcata cactattctc agaatgactt ggttgagtac tcaccagtca |
| 6841 | cagaaaagca tcttacggat ggcatgacag taagagaatt atgcagtgct gccataacca |
| 6901 | tgagtgataa cactgcggcc aacttacttc tgacaacgat cggaggaccg aaggagctaa |
| 6961 | ccgctttttt gcacaacatg ggggatcatg taactcgcct tgatcgttgg gaaccggagc |
| 7021 | tgaatgaagc cataccaaac gacgagcgtg acaccacgat gcctgtagca atggcaacaa |
| 7081 | cgttgcgcaa actattaact ggcgaactac ttactctagc ttcccggcaa caattaatag |
| 7141 | actggatgga ggcggataaa gttgcaggac cacttctgcg ctcggccctt ccggctggct |
| 7201 | ggtttattgc tgataaatct ggagccggtg agcgtgggtc tcgcggtatc attgcagcac |
| 7261 | tggggccaga tggtaagccc tcccgtatcg tagttatcta cacgacgggg agtcaggcaa |
| 7321 | ctatggatga acgaaataga cagatcgctg agataggtgc ctcactgatt aagcattggt |
| 7381 | aactgtcaga ccaagtttac tcatatatac tttagattga tttaaaactt catttttaat |
| 7441 | ttaaaaggat ctaggtgaag atcctttttg ataatctcat gaccaaaatc ccttaacgtg |
| 7501 | agttttcgtt ccactgagcg tcagaccccg tagaaaagat caaaggatct tcttgagatc |
| 7561 | ctttttttct gcgcgtaatc tgctgcttgc aaacaaaaaa accaccgcta ccagcggtgg |
| 7621 | tttgtttgcc ggatcaagag ctaccaactc tttttccgaa ggtaactggc ttcagcagag |
| 7681 | cgcagatacc aaatactgtc cttctagtgt agccgtagtt aggccaccac ttcaagaact |
| 7741 | ctgtagcacc gcctacatac ctcgctctgc taatcctgtt accagtggct gctgccagtg |
| 7801 | gcgataagtc gtgtcttacc gggttggact caagacgata gttaccggat aaggcgcagc |
| 7861 | ggtcgggctg aacggggggt tcgtgcacac agcccagctt ggagcgaacg acctacaccg |
| 7921 | aactgagata cctacagcgt gagctatgag aaagcgccac gcttcccgaa gggagaaagg |
| 7981 | cggacaggta tccggtaagc ggcagggtcg gaacaggaga gcgcacgagg gagcttccag |
| 8041 | ggggaaacgc ctggtatctt tatagtcctg tcgggtttcg ccacctctga cttgagcgtc |
| 8101 | gatttttgtg atgctcgtca ggggggcgga gcctatggaa aaacgccagc aacgcggcct |
| 8161 | ttttacggtt cctggccttt tgctggcctt ttgctcacat gttctttcct gcgttatccc |
| 8221 | ctgattctgt ggataaccgt attaccgcct ttgagtgagc tgataccgct cgccgcagcc |
| 8281 | gaacgaccga gcgcagcgag tcagtgagcg aggaagcgga agagcgccca atacgcaaac |
| 8341 | cgcctctccc cgcgcgttgg ccgattcatt aatgca |

What is claimed is:

1. A virus comprising a capsid protein and an optogenetic binding partner, wherein at least a portion of the optogenetic binding partner is displayed on the surface of the virus, wherein the optogenetic binding partner is linked to the capsid protein by a direct amino acid linkage or a linker.

2. The virus of claim 1, wherein the capsid protein comprises at least a portion of the amino acid sequence of VP1 (SEQ ID NO: 50).

3. The virus of claim 1, wherein the capsid protein comprises SEQ ID NO: 50, and wherein the optogenetic binding partner is inserted at M138 or G453 of SEQ ID NO: 50.

4. The virus of claim 1, wherein the optogenetic binding partner is selected from the group consisting of phytochrome interacting factor 1, phytochrome interacting factor 2, phytochrome interacting factor 3, phytochrome interacting factor 4, phytochrome interacting factor 5, and phytochrome interacting factor 6, portions thereof and variants thereof.

5. The virus of claim 1, wherein the optogenetic binding partner comprises the amino acid sequence of phytochrome interacting factor 1, a portion thereof or a variant thereof.

6. The virus of claim 1, wherein the amino acid sequence of the optogenetic binding partner is embedded within the amino acid sequence of the capsid protein.

7. The virus of claim 1, wherein the amino acid sequence of the optogenetic binding partner is adjacent to the amino acid sequence of the capsid protein.

8. The virus of claim 1, further comprising at least one linker between the N-terminus of the amino acid sequence of the optogenetic binding partner and the amino acid sequence of the capsid protein or between the C-terminus of the amino acid sequence and the amino acid sequence of the capsid protein.

9. The virus of claim 1, wherein the virus is an adeno-associated virus of serotype 2.

10. The virus of claim 1, wherein the capsid protein comprises SEQ ID NO: 50.

11. The virus of claim 1, further comprising a nucleic acid molecule selected from the group consisting of a gene, a portion of a gene, RNA interference and a CRISPR/Cas genome editing tool.

12. The virus of claim 1, further comprising an enzymatic cleavage motif adjacent to the optogenetic binding partner, wherein the enzymatic cleavage motif does not inactivate other biologically active motifs on the surface of the virus.

13. The virus of claim 12, wherein the enzymatic cleavage motif comprises an amino acid sequence that is cleavable by a protease selected from the group consisting of a matrix metalloprotease (MMP), an endopeptidase, a kinase, TEV protease, Cathepsin K (CTSK), a phosphatase and combinations thereof.

14. The virus of claim 12, wherein the enzymatic cleavage motif comprises an amino acid sequence that is cleavable by an endopeptidase.

15. The virus of claim 14, wherein the endopeptidase is enterokinase of SEQ ID NO: 76.

16. The virus of claim 12, wherein the enzymatic cleavage motif comprises an amino acid sequence selected from the group consisting of SEQ ID NO: 3 (DDDDK), SEQ ID NO: 176 (Glu-Asn-Leu-Tyr-Phe-Gln-Gly), SEQ ID NO: 17 (PLGLAR), SEQ ID NO: 2 (IPESLRAG), SEQ ID NO: 1 (IPVSLRSG), and SEQ ID NO: 18 (VPMSMRGG).

17. A method comprising:
providing an adeno-associated virus having one or more peptides genetically encoded into the capsid so as to be at least partially exposed to the surface of the capsid and a first enzymatic cleavage motif cleavable by an enzyme genetically encoded into the capsid adjacent to each of the one or more peptides;
treating the adeno-associated virus with said enzyme to cleave the first enzymatic cleavage motif, allowing at least a portion of the one or more peptides to be tethered to the capsid surface at either the C-terminal or N-terminal end to yield an enzyme-treated virus,
wherein at least one of the one or more peptides genetically encoded into the capsid is an optogenetic binding partner.

18. The method of claim 17, further comprising treating the enzyme-treated virus to remove the enzyme.

19. The method of claim 17, further comprising a step of administering the enzyme-treated virus to a target cell.

20. The method of claim 17, wherein the virus further comprises a second enzymatic cleavage motif adjacent to the one or more peptides at the opposite end of the one or more peptides from the first enzymatic cleavage motif.


SEQUENCE LISTING (177 sequences)

SEQ ID NO: 1 - protein, 8 aa, Artificial Sequence
  Description of Artificial Sequence Synthetic peptide
IPVSLRSG

SEQ ID NO: 2 - protein, 8 aa, Artificial Sequence
  Description of Artificial Sequence Synthetic peptide
IPESLRAG

SEQ ID NO: 3 - protein, 5 aa, Artificial Sequence
  Description of Artificial Sequence Synthetic peptide
DDDDK

SEQ ID NO: 4 - protein, 4 aa, Artificial Sequence
  Description of Artificial Sequence Synthetic peptide
GGGS

SEQ ID NO: 5 - protein, 7 aa, Artificial Sequence
  Description of Artificial Sequence Synthetic peptide
PKKKRKV

SEQ ID NO: 6 - protein, 21 aa, Artificial Sequence
  Description of Artificial Sequence Synthetic peptide
TRPQRDCPTPTWQPQPRRKSW

SEQ ID NO: 7 - protein, 11 aa, Artificial Sequence
  Description of Artificial Sequence Synthetic peptide
LQLPPLERLTL

SEQ ID NO: 8 - protein, 9 aa, Artificial Sequence
  Description of Artificial Sequence Synthetic peptide
LPPLERLTL

SEQ ID NO: 9 - protein, 18 aa, Artificial Sequence
  Description of Artificial Sequence Synthetic peptide
PSTRIQQQLGQLTLENLQ

SEQ ID NO: 10 - protein, 11 aa, Artificial Sequence
  Description of Artificial Sequence Synthetic peptide
MLALKLAGLDI

SEQ ID NO: 11 - DNA, 21 bp, Artificial Sequence
  Description of Artificial Sequence Synthetic oligonucleotide
cccaagaaaaagcggaaggtg

SEQ ID NO: 12 - DNA, 65 bp, Artificial Sequence
  Description of Artificial Sequence Synthetic oligonucleotide
acgaggccgcaaagagactgcccgacgccaacctggcagccgcagccaagaagaaaaagctggac

SEQ ID NO: 13 - DNA, 33 bp, Artificial Sequence
  Description of Artificial Sequence Synthetic oligonucleotide
cttcaacttcctcctcttgagagacttactctt

SEQ ID NO: 14 - DNA, 27 bp, Artificial Sequence
  Description of Artificial Sequence Synthetic oligonucleotide
cttcctcctcttgagagacttactctt

SEQ ID NO: 15 - DNA, 54 bp, Artificial Sequence
  Description of Artificial Sequence Synthetic oligonucleotide
cccagcacccggatccagcagcagctgggccagctgaccctggagaacctgcag

SEQ ID NO: 16 - DNA, 33 bp, Artificial Sequence
  Description of Artificial Sequence Synthetic oligonucleotide
atgttagccttgaaattagcaggtcttgatatc

SEQ ID NO: 17 - protein, 6 aa, Artificial Sequence
  Description of Artificial Sequence Synthetic peptide
PLGLAR

SEQ ID NO: 18 - protein, 8 aa, Artificial Sequence
  Description of Artificial Sequence Synthetic peptide
VPMSMRGG

SEQ ID NO: 19 - protein, 6 aa, Artificial Sequence
  Description of Artificial Sequence Synthetic peptide
  MOD_RES (6)..(6): Gln or Gly
ENLYFX

SEQ ID NO: 20 - protein, 4 aa, Artificial Sequence
  Description of Artificial Sequence Synthetic peptide
DDDK

SEQ ID NO: 21 - DNA, 21 bp, Artificial Sequence
  Description of Artificial Sequence Synthetic primer
tcacggggatttccaagtctc

SEQ ID NO: 22 - DNA, 22 bp, Artificial Sequence
  Description of Artificial Sequence Synthetic primer
aatggggcggagttgttacgac

SEQ ID NO: 23 - protein, 6 aa, Artificial Sequence
  Description of Artificial Sequence Synthetic 6xHis tag
HHHHHH

SEQ ID NO: 24 - DNA, 37 bp, Artificial Sequence
  Description of Artificial Sequence Synthetic primer
gcattaggtctctaatggtatctggtgttggtggttc

SEQ ID NO: 25 - DNA, 57 bp, Artificial Sequence
  Description of Artificial Sequence Synthetic primer
atgatgatgatgatgatgaccaccaccacctactgcaagagcttgttgtaattctgg

SEQ ID NO: 26 - DNA, 41 bp, Artificial Sequence
  Description of Artificial Sequence Synthetic primer
gctaatggtctcttttaatgatgatgatgatgatgaccacc

SEQ ID NO: 27 - protein, 15 aa, Artificial Sequence
  Description of Artificial Sequence Synthetic peptide
AGPLGLARGDDDKGA

SEQ ID NO: 28 - protein, 16 aa, Artificial Sequence
  Description of Artificial Sequence Synthetic peptide
AGDDDDKGPLGLARGA

SEQ ID NO: 29 - protein, 4 aa, Artificial Sequence
  Description of Artificial Sequence Synthetic peptide
DDDD

SEQ ID NO: 30 - protein, 18 aa, Artificial Sequence
  Description of Artificial Sequence Synthetic peptide
  MOD_RES (3)..(10): Any amino acid
  MOD_RES (12)..(16): Any amino acid
AGXXXXXXXXGXXXXXGA

SEQ ID NO: 31 - DNA, 1950 bp, Arabidopsis thaliana
atggtttccggagtcgggggtagtggcggtggccgtggcggtggccgtggcggagaagaagaaccgtcgtcaagtcacactcctaataaccgaagaggaggagaacaagctcaatcgtcgggaacgaaatctctcagaccaagaagcaacactgaatcaatgagcaaagcaattcaacagtacaccgtcgacgcaagactccacgccgttttcgaacaatccggcgaatcagggaaatcattcgactactcacaatcactcaaaacgacgacgtacggttcctctgtacctgagcaacagatcacagcttatctctctcgaatccagcgaggtggttacattcagcctttcggatgtatgatcgccgtcgatgaatccagtttccggatcatcggttacagtgaaaacgccagagaaatgttagggattatgcctcaatctgttcctactcttgagaaacctgagattctagctatgggaactgatgtgagatctttgttcacttcttcgagctcgattctactcgagcgtgctttcgttgctcgagagattaccttgttaaatccggtttggatccattccaagaatactggtaaaccgttttacgccattcttcataggattgatgttggtgttgttattgatttagagccagctagaactgaagatcctgcgctttctattgctggtgctgttcaatcgcagaaactcgcggttcgtgcgatttctcagttacaggctcttcctggtggagatattaagcttttgtgtgacactgtcgtggaaagtgtgagggacttgactggttatgatcgtgttatggtttataagtttcatgaagatgagcatggagaagttgtagctgagagtaaacgagatgatttagagccttatattggactgcattatcctgctactgatattcctcaagcgtcaaggttcttgtttaagcagaaccgtgtccgaatgatagtagattgcaatgccacacctgttcttgtggtccaggacgataggctaactcagtctatgtgcttggttggttctactcttagggctcctcatggttgtcactctcagtatatggctaacatgggatctattgcgtctttagcaatggcggttataatcaatggaaatgaagatgatgggagcaatgtagctagtggaagaagctcgatgaggctttggggtttggttgtttgccatcacacttcttctcgctgcataccgtttccgctaaggtatgcttgtgagtttttgatgcaggctttcggtttacagttaaacatggaattgcagttagctttgcaaatgtcagagaaacgcgttttgagaacgcagacactgttatgtgatatgcttctgcgtgactcgcctgctggaattgttacacagagtcccagtatcatggacttagtgaaatgtgacggtgcagcatttctttaccacgggaagtattacccgttgggtgttgctcctagtgaagttcagataaaagatgttgtggagtggttgcttgcgaatcatgcggattcaaccggattaagcactgatagtttaggcgatgcggggtatcccggtgcagctgcgttaggggatgctgtgtgcggtatggcagttgcatatatcacaaaaagagactttcttttttggtttcgatctcacactgcgaaagaaatcaaatggggaggcgctaagcatcatccggaggataaagatgatgggcaacgaatgcatcctcgttcgtcctttcaggcttttcttgaagttgttaagagccggagtcagccatgggaaactgcggaaatggatgcgattcactcgctccagcttattctgagagactcttttaaagaatctgaggcggctatgaactctaaagttgtggatggtgtggttcagccatgtagggatatggcgggggaacaggggattgatgagttaggt

SEQ ID NO: 32 - protein, 650 aa, Arabidopsis thaliana
MVSGVGGSGGGRGGGRGGEEEPSSSHTPNNRRGGEQAQSSGTKSLRPRSNTESMSKAIQQYTVDARLHAVFEQSGESGKSFDYSQSLKTTTYGSSVPEQQITAYLSRIQRGGYIQPFGCMIAVDESSFRIIGYSENAREMLGIMPQSVPTLEKPEILAMGTDVRSLFTSSSSILLERAFVAREITLLNPVWIHSKNTGKPFYAILHRIDVGVVIDLEPARTEDPALSIAGAVQSQKLAVRAISQLQALPGGDIKLLCDTVVESVRDLTGYDRVMVYKFHEDEHGEVVAESKRDDLEPYIGLHYPATDIPQASRFLFKQNRVRMIVDCNATPVLVVQDDRLTQSMCLVGSTLRAPHGCHSQYMANMGSIASLAMAVIINGNEDDGSNVASGRSSMRLWGLVVCHHTSSRCIPFPLRYACEFLMQAFGLQLNMELQLALQMSEKRVLRTQTLLCDMLLRDSPAGIVTQSPSIMDLVKCDGAAFLYHGKYYPLGVAPSEVQIKDVVEWLLANHADSTGLSTDSLGDAGYPGAAALGDAVCGMAVAYITKRDFLFWFRSHTAKEIKWGGAKHHPEDKDDGQRMHPRSSFQAFLEVVKSRSQPWETAEMDAIHSLQLILRDSFKESEAAMNSKVVDGVVQPCRDMAGEQGIDELG

SEQ ID NO: 33 - DNA, 2394 bp, Artificial Sequence
  Description of Artificial Sequence Synthetic polynucleotide
atggtttccggagtcgggggtagtggcggtggccgtggcggtggccgtggcggagaagaagaaccgtcgtcaagtcacactcctaataaccgaagaggaggagaacaagctcaatcgtcgggaacgaaatctctcagaccaagaagcaacactgaatcaatgagcaaagcaattcaacagtacaccgtcgacgcaagactccacgccgttttcgaacaatccggcgaatcagggaaatcattcgactactcacaatcactcaaaacgacgacgtacggttcctctgtacctgagcaacagatcacagcttatctctctcgaatccagcgaggtggttacattcagcctttcggatgtatgatcgccgtcgatgaatccagtttccggatcatcggttacagtgaaaacgccagagaaatgttagggattatgcctcaatctgttcctactcttgagaaacctgagattctagctatgggaactgatgtgagatctttgttcacttcttcgagctcgattctactcgagcgtgctttcgttgctcgagagattaccttgttaaatccggtttggatccattccaagaatactggtaaaccgttttacgccattcttcataggattgatgttggtgttgttattgatttagagccagctagaactgaagatcctgcgctttctattgctggtgctgttcaatcgcagaaactcgcggttcgtgcgatttctcagttacaggctcttcctggtggagatattaagcttttgtgtgacactgtcgtggaaagtgtgagggacttgactggttatgatcgtgttatggtttataagtttcatgaagatgagcatggagaagttgtagctgagagtaaacgagatgatttagagccttatattggactgcattatcctgctactgatattcctcaagcgtcaaggttcttgtttaagcagaaccgtgtccgaatgatagtagattgcaatgccacacctgttcttgtggtccaggacgataggctaactcagtctatgtgcttggttggttctactcttagggctcctcatggttgtcactctcagtatatggctaacatgggatctattgcgtctttagcaatggcggttataatcaatggaaatgaagatgatgggagcaatgtagctagtggaagaagctcgatgaggctttggggtttggttgtttgccatcacacttcttctcgctgcataccgtttccgctaaggtatgcttgtgagtttttgatgcaggctttcggtttacagttaaacatggaattgcagttagctttgcaaatgtcagagaaacgcgttttgagaacgcagacactgttatgtgatatgcttctgcgtgactcgcctgctggaattgttacacagagtcccagtatcatggacttagtgaaatgtgacggtgcagcatttctttaccacgggaagtattacccgttgggtgttgctcctagtgaagttcagataaaagatgttgtggagtggttgcttgcgaatcatgcggattcaaccggattaagcactgatagtttaggcgatgcggggtatcccggtgcagctgcgttaggggatgctgtgtgcggtatggcagttgcatatatcacaaaaagagactttcttttttggtttcgatctcacactgcgaaagaaatcaaatggggaggcgctaagcatcatccggaggataaagatgatgggcaacgaatgcatcctcgttcgtcctttcaggcttttcttgaagttgttaagagccggagtcagccatgggaaactgcggaaatggatgcgattcactcgctccagcttattctgagagactcttttaaagaatctgaggcggctatgaactctaaagttgtggatggtgtggttcagccatgtagggatatggcgggggaacaggggattgatgagttaggtgaattcgatagtgctggtagtgctggtagtgctggttccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggcccaagaaaaagcggaaggtg

SEQ ID NO: 34 - protein, 798 aa, Artificial Sequence
  Description of Artificial Sequence Synthetic polypeptide
MVSGVGGSGGGRGGGRGGEEEPSSSHTPNNRRGGEQAQSSGTKSLRPRSNTESMSKAIQQYTVDARLHAVFEQSGESGKSFDYSQSLKTTTYGSSVPEQQITAYLSRIQRGGYIQPFGCMIAVDESSFRIIGYSENAREMLGIMPQSVPTLEKPEILAMGTDVRSLFTSSSSILLERAFVAREITLLNPVWIHSKNTGKPFYAILHRIDVGVVIDLEPARTEDPALSIAGAVQSQKLAVRAISQLQALPGGDIKLLCDTVVESVRDLTGYDRVMVYKFHEDEHGEVVAESKRDDLEPYIGLHYPATDIPQASRFLFKQNRVRMIVDCNATPVLVVQDDRLTQSMCLVGSTLRAPHGCHSQYMANMGSIASLAMAVIINGNEDDGSNVASGRSSMRLWGLVVCHHTSSRCIPFPLRYACEFLMQAFGLQLNMELQLALQMSEKRVLRTQTLLCDMLLRDSPAGIVTQSPSIMDLVKCDGAAFLYHGKYYPLGVAPSEVQIKDVVEWLLANHADSTGLSTDSLGDAGYPGAAALGDAVCGMAVAYITKRDFLFWFRSHTAKEIKWGGAKHHPEDKDDGQRMHPRSSFQAFLEVVKSRSQPWETAEMDAIHSLQLILRDSFKESEAAMNSKVVDGVVQPCRDMAGEQGIDELGEFDSAGSAGSAGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGGPKKKRKV

SEQ ID NO: 35 - DNA, 2748 bp, Arabidopsis thaliana
gtatctggtgttggtggttctggtggtggaagaggtggaggtagaggaggtgaagaagaaccatcaagtagtcatacacctaacaatcgtagaggtggtgagcaagctcaatcatcaggtacaaaatcattacgtccaagaagtaatactgaatcaatgtcaaaagcaattcaacaatacacagtagatgctagattacacgccgtattcgaacaatctggagaaagtggtaagagttttgattactcacaatcattgaaaacaaccacttatggtagttcagttccagaacaacaaatcactgcatatcttagtagaatacaacgtggtggttacattcaaccatttggttgtatgattgcagttgatgaatcttcttttagaatcattggttattcagaaaatgcaagagaaatgttgggtatcatgccacaatcagtaccaaccttagaaaaaccagaaattcttgcaatgggtacagatgttagaagtttgtttacatcatcatcatcaattcttttggagagagcttttgttgcacgtgaaatcactttacttaatccagtatggattcatagtaagaatactggaaagccattctatgcaattcttcatagaatagatgtaggagttgttattgatcttgagccagcaagaacagaagatccagcattatctattgctggtgcagtacaatcacaaaaacttgctgttagagcaattagtcaattacaagccttgccaggtggtgatataaaacttctttgtgatacagttgttgaatcagttcgtgatcttaccggttatgatagagttatggtatacaaattccatgaggatgaacatggtgaagttgttgcagaaagtaaaagagatgatcttgaaccatacattggtttgcattatccagctactgatattccacaagcatcaagatttcttttcaaacaaaatcgtgttagaatgattgtagattgtaatgccaccccagtattagttgttcaagatgatagattgacacaaagtatgtgtttagtaggttcaacattaagagcacctcatggatgtcattcacaatatatggccaatatgggttcaatagcatcattagctatggcagtaatcatcaatggaaatgaagatgatggttcaaatgttgcatcaggtagaagttcaatgcgtttatggggtttagtagtttgtcatcatacaagttctcgttgtatcccatttcctttacgttatgcatgtgaatttcttatgcaagcatttggtttacaattgaatatggaacttcaattagcattacaaatgagtgaaaagagagttttacgtacacaaacattgttatgcgatatgttattgagagattctccagctggtattgttactcaatcaccatctatcatggatcttgtaaagtgtgatggtgcagcattcttataccacggaaagtactatccattaggtgttgcaccatctgaagttcaaatcaaagatgttgtagaatggttattggctaatcacgcagattctactggtttatcaactgattctcttggtgatgctggttatcctggtgccgcagccttaggagatgctgtatgtggtatggccgttgcttacattacaaaaagagatttcttgttttggtttcgttctcatacagctaaagagatcaaatggggtggtgcaaaacatcatccagaagataaggatgatggtcaaagaatgcatccaagatcatcatttcaagcattcttagaagtagttaagtcaagaagtcaaccttgggaaacagcagaaatggatgcaatacattcattacaattgatacttcgtgattcattcaaagaatcagaagcagcaatgaatagtaaagttgttgatggtgttgttcaaccatgtagagatatggccggtgaacaaggtattgatgaattaggtgctgtagctagagaaatggttagattgatagaaactgccactgttccaatcttcgctgttgatgctggtggatgcataaacggttggaatgctaagatcgcagaattgaccggtttgtcagttgaagaagctatgggtaaaagtttagtttcagatttgatctataaggaaaatgaagcaaccgttaacaaattgttatcaagagcattgagaggagatgaggaaaagaatgtagaagttaagttaaagacattttcaccagagttacaaggtaaagcagtttttgttgtagttaatgcttgttcatcaaaagattacttgaataacattgtaggtgtttgttttgttggtcaagatgtaacttcacaaaagattgttatggataagtttatcaatatccaaggtgattacaaagctattgttcattctccaaatccattgattccaccaatctttgcagctgatgagaatacatgttgtttagaatggaatatggcaatggaaaagttaactggttggtcacgttcagaagtaattggtaagatgattgttggagaggtttttggtagttgttgtatgcttaaaggtccagatgctttaactaagtttatgattgttttgcataatgcaattggtggtcaagatacagataagttcccattccctttcttcgatagaaatggaaagtttgttcaagcattacttactgctaacaaaagagtatcattagaaggtaaagtaataggagctttttgtttcttacaaattccttcaccagaattacaacaagctcttgcagta

SEQ ID NO: 36 - protein, 916 aa, Arabidopsis thaliana
VSGVGGSGGGRGGGRGGEEEPSSSHTPNNRRGGEQAQSSGTKSLRPRSNTESMSKAIQQYTVDARLHAVFEQSGESGKSFDYSQSLKTTTYGSSVPEQQITAYLSRIQRGGYIQPFGCMIAVDESSFRIIGYSENAREMLGIMPQSVPTLEKPEILAMGTDVRSLFTSSSSILLERAFVAREITLLNPVWIHSKNTGKPFYAILHRIDVGVVIDLEPARTEDPALSIAGAVQSQKLAVRAISQLQALPGGDIKLLCDTVVESVRDLTGYDRVMVYKFHEDEHGEVVAESKRDDLEPYIGLHYPATDIPQASRFLFKQNRVRMIVDCNATPVLVVQDDRLTQSMCLVGSTLRAPHGCHSQYMANMGSIASLAMAVIINGNEDDGSNVASGRSSMRLWGLVVCHHTSSRCIPFPLRYACEFLMQAFGLQLNMELQLALQMSEKRVLRTQTLLCDMLLRDSPAGIVTQSPSIMDLVKCDGAAFLYHGKYYPLGVAPSEVQIKDVVEWLLANHADSTGLSTDSLGDAGYPGAAALGDAVCGMAVAYITKRDFLFWFRSHTAKEIKWGGAKHHPEDKDDGQRMHPRSSFQAFLEVVKSRSQPWETAEMDAIHSLQLILRDSFKESEAAMNSKVVDGVVQPCRDMAGEQGIDELGAVAREMVRLIETATVPIFAVDAGGCINGWNAKIAELTGLSVEEAMGKSLVSDLIYKENEATVNKLLSRALRGDEEKNVEVKLKTFSPELQGKAVFVVVNACSSKDYLNNIVGVCFVGQDVTSQKIVMDKFINIQGDYKAIVHSPNPLIPPIFAADENTCCLEWNMAMEKLTGWSRSEVIGKMIVGEVFGSCCMLKGPDALTKFMIVLHNAIGGQDTDKFPFPFFDRNGKFVQALLTANKRVSLEGKVIGAFCFLQIPSPELQQALAV

SEQ ID NO: 37 - DNA, 2778 bp, Artificial Sequence
  Description of Artificial Sequence Synthetic polynucleotide
gtatctggtgttggtggttctggtggtggaagaggtggaggtagaggaggtgaagaagaaccatcaagtagtcatacacctaacaatcgtagaggtggtgagcaagctcaatcatcaggtacaaaatcattacgtccaagaagtaatactgaatcaatgtcaaaagcaattcaacaatacacagtagatgctagattacacgccgtattcgaacaatctggagaaagtggtaagagttttgattactcacaatcattgaaaacaaccacttatggtagttcagttccagaacaacaaatcactgcatatcttagtagaatacaacgtggtggttacattcaaccatttggttgtatgattgcagttgatgaatcttcttttagaatcattggttattcagaaaatgcaagagaaatgttgggtatcatgccacaatcagtaccaaccttagaaaaaccagaaattcttgcaatgggtacagatgttagaagtttgtttacatcatcatcatcaattcttttggagagagcttttgttgcacgtgaaatcactttacttaatccagtatggattcatagtaagaatactggaaagccattctatgcaattcttcatagaatagatgtaggagttgttattgatcttgagccagcaagaacagaagatccagcattatctattgctggtgcagtacaatcacaaaaacttgctgttagagcaattagtcaattacaagccttgccaggtggtgatataaaacttctttgtgatacagttgttgaatcagttcgtgatcttaccggttatgatagagttatggtatacaaattccatgaggatgaacatggtgaagttgttgcagaaagtaaaagagatgatcttgaaccatacattggtttgcattatccagctactgatattccacaagcatcaagatttcttttcaaacaaaatcgtgttagaatgattgtagattgtaatgccaccccagtattagttgttcaagatgatagattgacacaaagtatgtgtttagtaggttcaacattaagagcacctcatggatgtcattcacaatatatggccaatatgggttcaatagcatcattagctatggcagtaatcatcaatggaaatgaagatgatggttcaaatgttgcatcaggtagaagttcaatgcgtttatggggtttagtagtttgtcatcatacaagttctcgttgtatcccatttcctttacgttatgcatgtgaatttcttatgcaagcatttggtttacaattgaatatggaacttcaattagcattacaaatgagtgaaaagagagttttacgtacacaaacattgttatgcgatatgttattgagagattctccagctggtattgttactcaatcaccatctatcatggatcttgtaaagtgtgatggtgcagcattcttataccacggaaagtactatccattaggtgttgcaccatctgaagttcaaatcaaagatgttgtagaatggttattggctaatcacgcagattctactggtttatcaactgattctcttggtgatgctggttatcctggtgccgcagccttaggagatgctgtatgtggtatggccgttgcttacattacaaaaagagatttcttgttttggtttcgttctcatacagctaaagagatcaaatggggtggtgcaaaacatcatccagaagataaggatgatggtcaaagaatgcatccaagatcatcatttcaagcattcttagaagtagttaagtcaagaagtcaaccttgggaaacagcagaaatggatgcaatacattcattacaattgatacttcgtgattcattcaaagaatcagaagcagcaatgaatagtaaagttgttgatggtgttgttcaaccatgtagagatatggccggtgaacaaggtattgatgaattaggtgctgtagctagagaaatggttagattgatagaaactgccactgttccaatcttcgctgttgatgctggtggatgcataaacggttggaatgctaagatcgcagaattgaccggtttgtcagttgaagaagctatgggtaaaagtttagtttcagatttgatctataaggaaaatgaagcaaccgttaacaaattgttatcaagagcattgagaggagatgaggaaaagaatgtagaagttaagttaaagacattttcaccagagttacaaggtaaagcagtttttgttgtagttaatgcttgttcatcaaaagattacttgaataacattgtaggtgtttgttttgttggtcaagatgtaacttcacaaaagattgttatggataagtttatcaatatccaaggtgattacaaagctattgttcattctccaaatccattgattccaccaatctttgcagctgatgagaatacatgttgtttagaatggaatatggcaatggaaaagttaactggttggtcacgttcagaagtaattggtaagatgattgttggagaggtttttggtagttgttgtatgcttaaaggtccagatgctttaactaagtttatgattgttttgcataatgcaattggtggtcaagatacagataagttcccattccctttcttcgatagaaatggaaagtttgttcaagcattacttactgctaacaaaagagtatcattagaaggtaaagtaataggagctttttgtttcttacaaattccttcaccagaattacaacaagctcttgcagtaggtgcttcaggtcatcatcatcatcatcat

SEQ ID NO: 38 - protein, 926 aa, Artificial Sequence
  Description of Artificial Sequence Synthetic polypeptide
VSGVGGSGGGRGGGRGGEEEPSSSHTPNNRRGGEQAQSSGTKSLRPRSNTESMSKAIQQYTVDARLHAVFEQSGESGKSFDYSQSLKTTTYGSSVPEQQITAYLSRIQRGGYIQPFGCMIAVDESSFRIIGYSENAREMLGIMPQSVPTLEKPEILAMGTDVRSLFTSSSSILLERAFVAREITLLNPVWIHSKNTGKPFYAILHRIDVGVVIDLEPARTEDPALSIAGAVQSQKLAVRAISQLQALPGGDIKLLCDTVVESVRDLTGYDRVMVYKFHEDEHGEVVAESKRDDLEPYIGLHYPATDIPQASRFLFKQNRVRMIVDCNATPVLVVQDDRLTQSMCLVGSTLRAPHGCHSQYMANMGSIASLAMAVIINGNEDDGSNVASGRSSMRLWGLVVCHHTSSRCIPFPLRYACEFLMQAFGLQLNMELQLALQMSEKRVLRTQTLLCDMLLRDSPAGIVTQSPSIMDLVKCDGAAFLYHGKYYPLGVAPSEVQIKDVVEWLLANHADSTGLSTDSLGDAGYPGAAALGDAVCGMAVAYITKRDFLFWFRSHTAKEIKWGGAKHHPEDKDDGQRMHPRSSFQAFLEVVKSRSQPWETAEMDAIHSLQLILRDSFKESEAAMNSKVVDGVVQPCRDMAGEQGIDELGAVAREMVRLIETATVPIFAVDAGGCINGWNAKIAELTGLSVEEAMGKSLVSDLIYKENEATVNKLLSRALRGDEEKNVEVKLKTFSPELQGKAVFVVVNACSSKDYLNNIVGVCFVGQDVTSQKIVMDKFINIQGDYKAIVHSPNPLIPPIFAADENTCCLEWNMAMEKLTGWSRSEVIGKMIVGEVFGSCCMLKGPDALTKFMIVLHNAIGGQDTDKFPFPFFDRNGKFVQALLTANKRVSLEGKVIGAFCFLQIPSPELQQALAVGASGHHHHHH

SEQ ID NO: 39 - DNA, 2772 bp, Arabidopsis thaliana
atgggtgcttcaggtgtatctggtgttggtggttctggtggtggaagaggtggaggtagaggaggtgaagaagaaccatcaagtagtcatacacctaacaatcgtagaggtggtgagcaagctcaatcatcaggtacaaaatcattacgtccaagaagtaatactgaatcaatgtcaaaagcaattcaacaatacacagtagatgctagattacacgccgtattcgaacaatctggagaaagtggtaagagttttgattactcacaatcattgaaaacaaccacttatggtagttcagttccagaacaacaaatcactgcatatcttagtagaatacaacgtggtggttacattcaaccatttggttgtatgattgcagttgatgaatcttcttttagaatcattggttattcagaaaatgcaagagaaatgttgggtatcatgccacaatcagtaccaaccttagaaaaaccagaaattcttgcaatgggtacagatgttagaagtttgtttacatcatcatcatcaattcttttggagagagcttttgttgcacgtgaaatcactttacttaatccagtatggattcatagtaagaatactggaaagccattctatgcaattcttcatagaatagatgtaggagttgttattgatcttgagccagcaagaacagaagatccagcattatctattgctggtgcagtacaatcacaaaaacttgctgttagagcaattagtcaattacaagccttgccaggtggtgatataaaacttctttgtgatacagttgttgaatcagttcgtgatcttaccggttatgatagagttatggtatacaaattccatgaggatgaacatggtgaagttgttgcagaaagtaaaagagatgatcttgaaccatacattggtttgcattatccagctactgatattccacaagcatcaagatttcttttcaaacaaaatcgtgttagaatgattgtagattgtaatgccaccccagtattagttgttcaagatgatagattgacacaaagtatgtgtttagtaggttcaacattaagagcacctcatggatgtcattcacaatatatggccaatatgggttcaatagcatcattagctatggcagtaatcatcaatggaaatgaagatgatggttcaaatgttgcatcaggtagaagttcaatgcgtttatggggtttagtagtttgtcatcatacaagttctcgttgtatcccatttcctttacgttatgcatgtgaatttcttatgcaagcatttggtttacaattgaatatggaacttcaattagcattacaaatgagtgaaaagagagttttacgtacacaaacattgttatgcgatatgttattgagagattctccagctggtattgttactcaatcaccatctatcatggatcttgtaaagtgtgatggtgcagcattcttataccacggaaagtactatccattaggtgttgcaccatctgaagttcaaatcaaagatgttgtagaatggttattggctaatcacgcagattctactggtttatcaactgattctcttggtgatgctggttatcctggtgccgcagccttaggagatgctgtatgtggtatggccgttgcttacattacaaaaagagatttcttgttttggtttcgttctcatacagctaaagagatcaaatggggtggtgcaaaacatcatccagaagataaggatgatggtcaaagaatgcatccaagatcatcatttcaagcattcttagaagtagttaagtcaagaagtcaaccttgggaaacagcagaaatggatgcaatacattcattacaattgatacttcgtgattcattcaaagaatcagaagcagcaatgaatagtaaagttgttgatggtgttgttcaaccatgtagagatatggccggtgaacaaggtattgatgaattaggtgctgtagctagagaaatggttagattgatagaaactgccactgttccaatcttcgctgttgatgctggtggatgcataaacggttggaatgctaagatcgcagaattgaccggtttgtcagttgaagaagctatgggtaaaagtttagtttcagatttgatctataaggaaaatgaagcaaccgttaacaaattgttatcaagagcattgagaggagatgaggaaaagaatgtagaagttaagttaaagacattttcaccagagttacaaggtaaagcagtttttgttgtagttaatgcttgttcatcaaaagattacttgaataacattgtaggtgtttgttttgttggtcaagatgtaacttcacaaaagattgttatggataagtttatcaatatccaaggtgattacaaagctattgttcattctccaaatccattgattccaccaatctttgcagctgatgagaatacatgttgtttagaatggaatatggcaatggaaaagttaactggttggtcacgttcagaagtaattggtaagatgattgttggagaggtttttggtagttgttgtatgcttaaaggtccagatgctttaactaagtttatgattgttttgcataatgcaattggtggtcaagatacagataagttcccattccctttcttcgatagaaatggaaagtttgttcaagcattacttactgctaacaaaagagtatcattagaaggtaaagtaataggagctttttgtttcttacaaattccttcaccagaattacaacaagctcttgcagtaggtggtagt

SEQ ID NO: 40 - protein, 924 aa, Arabidopsis thaliana
MGASGVSGVGGSGGGRGGGRGGEEEPSSSHTPNNRRGGEQAQSSGTKSLRPRSNTESMSKAIQQYTVDARLHAVFEQSGESGKSFDYSQSLKTTTYGSSVPEQQITAYLSRIQRGGYIQPFGCMIAVDESSFRIIGYSENAREMLGIMPQSVPTLEKPEILAMGTDVRSLFTSSSSILLERAFVAREITLLNPVWIHSKNTGKPFYAILHRIDVGVVIDLEPARTEDPALSIAGAVQSQKLAVRAISQLQALPGGDIKLLCDTVVESVRDLTGYDRVMVYKFHEDEHGEVVAESKRDDLEPYIGLHYPATDIPQASRFLFKQNRVRMIVDCNATPVLVVQDDRLTQSMCLVGSTLRAPHGCHSQYMANMGSIASLAMAVIINGNEDDGSNVASGRSSMRLWGLVVCHHTSSRCIPFPLRYACEFLMQAFGLQLNMELQLALQMSEKRVLRTQTLLCDMLLRDSPAGIVTQSPSIMDLVKCDGAAFLYHGKYYPLGVAPSEVQIKDVVEWLLANHADSTGLSTDSLGDAGYPGAAALGDAVCGMAVAYITKRDFLFWFRSHTAKEIKWGGAKHHPEDKDDGQRMHPRSSFQAFLEVVKSRSQPWETAEMDAIHSLQLILRDSFKESEAAMNSKVVDGVVQPCRDMAGEQGIDELGAVAREMVRLIETATVPIFAVDAGGCINGWNAKIAELTGLSVEEAMGKSLVSDLIYKENEATVNKLLSRALRGDEEKNVEVKLKTFSPELQGKAVFVVVNACSSKDYLNNIVGVCFVGQDVTSQKIVMDKFINIQGDYKAIVHSPNPLIPPIFAADENTCCLEWNMAMEKLTGWSRSEVIGKMIVGEVFGSCCMLKGPDALTKFMIVLHNAIGGQDTDKFPFPFFDRNGKFVQALLTANKRVSLEGKVIGAFCFLQIPSPELQQALAVGGS

SEQ ID NO: 41 - DNA, 2793 bp, Artificial Sequence
  Description of Artificial Sequence Synthetic polynucleotide
atgggtgcttcaggtgtatctggtgttggtggttctggtggtggaagaggtggaggtagaggaggtgaagaagaaccatcaagtagtcatacacctaacaatcgtagaggtggtgagcaagctcaatcatcaggtacaaaatcattacgtccaagaagtaatactgaatcaatgtcaaaagcaattcaacaatacacagtagatgctagattacacgccgtattcgaacaatctggagaaagtggtaagagttttgattactcacaatcattgaaaacaaccacttatggtagttcagttccagaacaacaaatcactgcatatcttagtagaatacaacgtggtggttacattcaaccatttggttgtatgattgcagttgatgaatcttcttttagaatcattggttattcagaaaatgcaagagaaatgttgggtatcatgccacaatcagtaccaaccttagaaaaaccagaaattcttgcaatgggtacagatgttagaagtttgtttacatcatcatcatcaattcttttggagagagcttttgttgcacgtgaaatcactttacttaatccagtatggattcatagtaagaatactggaaagccattctatgcaattcttcatagaatagatgtaggagttgttattgatcttgagccagcaagaacagaagatccagcattatctattgctggtgcagtacaatcacaaaaacttgctgttagagcaattagtcaattacaagccttgccaggtggtgatataaaacttctttgtgatacagttgttgaatcagttcgtgatcttaccggttatgatagagttatggtatacaaattccatgaggatgaacatggtgaagttgttgcagaaagtaaaagagatgatcttgaaccatacattggtttgcattatccagctactgatattccacaagcatcaagatttcttttcaaacaaaatcgtgttagaatgattgtagattgtaatgccaccccagtattagttgttcaagatgatagattgacacaaagtatgtgtttagtaggttcaacattaagagcacctcatggatgtcattcacaatatatggccaatatgggttcaatagcatcattagctatggcagtaatcatcaatggaaatgaagatgatggttcaaatgttgcatcaggtagaagttcaatgcgtttatggggtttagtagtttgtcatcatacaagttctcgttgtatcccatttcctttacgttatgcatgtgaatttcttatgcaagcatttggtttacaattgaatatggaacttcaattagcattacaaatgagtgaaaagagagttttacgtacacaaacattgttatgcgatatgttattgagagattctccagctggtattgttactcaatcaccatctatcatggatcttgtaaagtgtgatggtgcagcattcttataccacggaaagtactatccattaggtgttgcaccatctgaagttcaaatcaaagatgttgtagaatggttattggctaatcacgcagattctactggtttatcaactgattctcttggtgatgctggttatcctggtgccgcagccttaggagatgctgtatgtggtatggccgttgcttacattacaaaaagagatttcttgttttggtttcgttctcatacagctaaagagatcaaatggggtggtgcaaaacatcatccagaagataaggatgatggtcaaagaatgcatccaagatcatcatttcaagcattcttagaagtagttaagtcaagaagtcaaccttgggaaacagcagaaatggatgcaatacattcattacaattgatacttcgtgattcattcaaagaatcagaagcagcaatgaatagtaaagttgttgatggtgttgttcaaccatgtagagatatggccggtgaacaaggtattgatgaattaggtgctgtagctagagaaatggttagattgatagaaactgccactgttccaatcttcgctgttgatgctggtggatgcataaacggttggaatgctaagatcgcagaattgaccggtttgtcagttgaagaagctatgggtaaaagtttagtttcagatttgatctataaggaaaatgaagcaaccgttaacaaattgttatcaagagcattgagaggagatgaggaaaagaatgtagaagttaagttaaagacattttcaccagagttacaaggtaaagcagtttttgttgtagttaatgcttgttcatcaaaagattacttgaataacattgtaggtgtttgttttgttggtcaagatgtaacttcacaaaagattgttatggataagtttatcaatatccaaggtgattacaaagctattgttcattctccaaatccattgattccaccaatctttgcagctgatgagaatacatgttgtttagaatggaatatggcaatggaaaagttaactggttggtcacgttcagaagtaattggtaagatgattgttggagaggtttttggtagttgttgtatgcttaaaggtccagatgctttaactaagtttatgattgttttgcataatgcaattggtggtcaagatacagataagttcccattccctttcttcgatagaaatggaaagtttgttcaagcattacttactgctaacaaaagagtatcattagaaggtaaagtaataggagctttttgtttcttacaaattccttcaccagaattacaacaagctcttgcagtaggtggtagtcatcatcatcatcatcattaa

SEQ ID NO: 42 - protein, 930 aa, Artificial Sequence
  Description of Artificial Sequence Synthetic polypeptide
MGASGVSGVGGSGGGRGGGRGGEEEPSSSHTPNNRRGGEQAQSSGTKSLRPRSNTESMSKAIQQYTVDARLHAVFEQSGESGKSFDYSQSLKTTTYGSSVPEQQITAYLSRIQRGGYIQPFGCMIAVDESSFRIIGYSENAREMLGIMPQSVPTLEKPEILAMGTDVRSLFTSSSSILLERAFVAREITLLNPVWIHSKNTGKPFYAILHRIDVGVVIDLEPARTEDPALSIAGAVQSQKLAVRAISQLQALPGGDIKLLCDTVVESVRDLTGYDRVMVYKFHEDEHGEVVAESKRDDLEPYIGLHYPATDIPQASRFLFKQNRVRMIVDCNATPVLVVQDDRLTQSMCLVGSTLRAPHGCHSQYMANMGSIASLAMAVIINGNEDDGSNVASGRSSMRLWGLVVCHHTSSRCIPFPLRYACEFLMQAFGLQLNMELQLALQMSEKRVLRTQTLLCDMLLRDSPAGIVTQSPSIMDLVKCDGAAFLYHGKYYPLGVAPSEVQIKDVVEWLLANHADSTGLSTDSLGDAGYPGAAALGDAVCGMAVAYITKRDFLFWFRSHTAKEIKWGGAKHHPEDKDDGQRMHPRSSFQAFLEVVKSRSQPWETAEMDAIHSLQLILRDSFKESEAAMNSKVVDGVVQPCRDMAGEQGIDELGAVAREMVRLIETATVPIFAVDAGGCINGWNAKIAELTGLSVEEAMGKSLVSDLIYKENEATVNKLLSRALRGDEEKNVEVKLKTFSPELQGKAVFVVVNACSSKDYLNNIVGVCFVGQDVTSQKIVMDKFINIQGDYKAIVHSPNPLIPPIFAADENTCCLEWNMAMEKLTGWSRSEVIGKMIVGEVFGSCCMLKGPDALTKFMIVLHNAIGGQDTDKFPFPFFDRNGKFVQALLTANKRVSLEGKVIGAFCFLQIPSPELQQALAVGGSHHHHHH

SEQ ID NO: 43 - DNA, 2520 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagatggccggcatgatgttccttcctactgattattgttgcagactgagcgaccaggaatacatggaactcgtcttcgagaacggacagatactcgcaaaaggccagaggtcaaatgttagtctccataatcagcggacgaaaagcatcatggatctgtatgaggccgaatacaacgaagattttatgaaaagtattatccatggagggggtggcgctattaccaacctgggagatacccaagtggtcccacagtcccacgtagcagccgctcacgagaccaatatgctggagtccaacaaacacgtagacggcgccgctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgctggctacaggcagtggcgcaccactggcagacaataacgagggcgccgacggagtgggtaattcctcgggaaattggcattgcgattccacatggctgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 44 - protein, 839 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKMAGMMFLPTDYCCRLSDQEYMELVFENGQILAKGQRSNVSLHNQRTKSIMDLYEAEYNEDFMKSIIHGGGGAITNLGDTQVVPQSHVAAAHETNMLESNKHVDGAAPGKKRPVEHSPVEPDSSSGTGKAGQQPARKRLNFGQTGDADSVPDPQPLGQPPAAPSGLGTNTLATGSGAPLADNNEGADGVGNSSGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 45 - DNA, 2109 bp, Artificial Sequence
  Description of Artificial Sequence Synthetic polynucleotide
atggccggcatgatgttccttcctactgattattgttgcagactgagcgaccaggaatacatggaactcgtcttcgagaacggacagatactcgcaaaaggccagaggtcaaatgttagtctccataatcagcggacgaaaagcatcatggatctgtatgaggccgaatacaacgaagattttatgaaaagtattatccatggagggggtggcgctattaccaacctgggagatacccaagtggtcccacagtcccacgtagcagccgctcacgagaccaatatgctggagtccaacaaacacgtagacggcgccgctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgctggctacaggcagtggcgcaccactggcagacaataacgagggcgccgacggagtgggtaattcctcgggaaattggcattgcgattccacatggctgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 46 - protein, 702 aa, Artificial Sequence
  Description of Artificial Sequence Synthetic polypeptide
MAGMMFLPTDYCCRLSDQEYMELVFENGQILAKGQRSNVSLHNQRTKSIMDLYEAEYNEDFMKSIIHGGGGAITNLGDTQVVPQSHVAAAHETNMLESNKHVDGAAPGKKRPVEHSPVEPDSSSGTGKAGQQPARKRLNFGQTGDADSVPDPQPLGQPPAAPSGLGTNTLATGSGAPLADNNEGADGVGNSSGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 47 - DNA, 312 bp, Artificial Sequence
  Description of Artificial Sequence Synthetic polynucleotide
gccggcatgatgttccttcctactgattattgttgcagactgagcgaccaggaatacatggaactcgtcttcgagaacggacagatactcgcaaaaggccagaggtcaaatgttagtctccataatcagcggacgaaaagcatcatggatctgtatgaggccgaatacaacgaagattttatgaaaagtattatccatggagggggtggcgctattaccaacctgggagatacccaagtggtcccacagtcccacgtagcagccgctcacgagaccaatatgctggagtccaacaaacacgtagacggcgcc

SEQ ID NO: 48 - protein, 104 aa, Artificial Sequence
  Description of Artificial Sequence Synthetic polypeptide
AGMMFLPTDYCCRLSDQEYMELVFENGQILAKGQRSNVSLHNQRTKSIMDLYEAEYNEDFMKSIIHGGGGAITNLGDTQVVPQSHVAAAHETNMLESNKHVDGA

SEQ ID NO: 49 - DNA, 2208 bp, Adeno-associated virus 2
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagaaggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaataccatggctacaggcagtggcgcaccaatggcagacaataacgagggtgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggctatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtgcctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 50 - protein, 735 aa, Adeno-associated virus 2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKKAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 51 - DNA, 1797 bp, Adeno-associated virus 2
aaggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaataccatggctacaggcagtggcgcaccaatggcagacaataacgagggtgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggctatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtgcctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 52 - protein, 598 aa, Adeno-associated virus 2
KAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 53 - DNA, 1602 bp, Adeno-associated virus 2
atggctacaggcagtggcgcaccaatggcagacaataacgagggtgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggctatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtgcctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 54 - protein, 533 aa, Adeno-associated virus 2
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 55 - DNA, 21 bp, Artificial Sequence
  Description of Artificial Sequence Synthetic oligonucleotide
cccaagaaaaagcggaaggtg

SEQ ID NO: 56 - protein, 7 aa, Artificial Sequence
  Description of Artificial Sequence Synthetic peptide
PKKKRKV

SEQ ID NO: 57 - DNA, 65 bp, Artificial Sequence; identical to SEQ ID NO: 12
  Description of Artificial Sequence Synthetic oligonucleotide

SEQ ID NO: 58 - protein, 21 aa, Artificial Sequence
  Description of Artificial Sequence Synthetic peptide
TRPQRDCPTPTWQPQPRRKSW

SEQ ID NO: 59 - DNA, 33 bp, Human immunodeficiency virus; identical to SEQ ID NO: 13

SEQ ID NO: 60 - protein, 11 aa, Human immunodeficiency virus
LQLPPLERLTL

SEQ ID NO: 61 - DNA, 27 bp, Human immunodeficiency virus
cttcctcctcttgagagacttactctt

SEQ ID NO: 62 - protein, 9 aa, Human immunodeficiency virus
LPPLERLTL

SEQ ID NO: 63 - DNA, 54 bp, Artificial Sequence; identical to SEQ ID NO: 15
  Description of Artificial Sequence Synthetic oligonucleotide

SEQ ID NO: 64 - protein, 18 aa, Artificial Sequence
  Description of Artificial Sequence Synthetic peptide
PSTRIQQQLGQLTLENLQ

SEQ ID NO: 65 - DNA, 33 bp, Artificial Sequence; identical to SEQ ID NO: 16
  Description of Artificial Sequence Synthetic oligonucleotide

SEQ ID NO: 66 - protein, 11 aa, Artificial Sequence
  Description of Artificial Sequence Synthetic peptide
MLALKLAGLDI

SEQ ID NO: 67 - DNA, 408 bp, Avena sativa
ttggctactacacttgaacgtattgagaagaactttgtcattactgacccaagattgccagataatcccattatattcgcgtccgatagtttcttgcagttgacagaatatagccgtgaagaaattttgggaagaaactgcaggtttctacaaggtcctgaaactgatcgcgcgacagtgagaaaaattagagatgccatagataaccaaacagaggtcactgttcagctgattaattatacaaagagtggtaaaaagttctggaacctctttcacttgcagcctatgcgagatcagaagggagatgtccagtactttattggggttcagttggatggaactgagcatgtccgagatgctgccgagagagagggagtcatgctgattaagaaaactgcagaaaatatt

SEQ ID NO: 68 - protein, 136 aa, Avena sativa
LATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENI

SEQ ID NO: 69 - DNA, 18930 bp, Artificial Sequence
  Description of Artificial Sequence Synthetic polynucleotide
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttaaggtgcacggcccacgtggccactagtacttctcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatcctctagttaattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 70 - DNA, 8376 bp, Artificial Sequence
  Description of Artificial Sequence Synthetic polynucleotide
aattcccatcatcaataatataccttattttggattgaagccaatatgataatgagggggtggagtttgtgacgtggcgcggggcgtgggaacggggcgggtgacgtagtagtctctagagtcctgtattagaggtcacgtgagtgttttgcgacattttgcgacaccatgtggtcacgctgggtatttaagcccgagtgagcacgcagggtctccattttgaagcgggaggtttgaacgcgcagccaccacgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatgctctagagtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgca

SEQ ID NO: 71 - protein, 621 aa, Artificial Sequence
  Description of Artificial Sequence Synthetic polypeptide
TPGFYEIVIKVPSDLDGHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 72 - protein, 735 aa, Artificial Sequence [sequence omitted]

SEQ ID NO: 73 - DNA, 7582 bp, Artificial Sequence
  Description of Artificial Sequence Synthetic polynucleotide
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagatggccggcatgatgttccttcctactgattattgttgcagactgagcgaccaggaatacatggaactcgtcttcgagaacggacagatactcgcaaaaggccagaggtcaaatgttagtctccataatcagcggacgaaaagcatcatggatctgtatgaggccgaatacaacgaagattttatgaaaagtattatccatggagggggtggcgctattaccaacctgggagatacccaagtggtcccacagtcccacgtagcagccgctcacgagaccaatatgctggagtccaacaaacacgtagacggcgccgctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgctggctacaggcagtggcgcaccactggcagacaataacgagggcgccgacggagtgggtaattcctcgggaaattggcattgcgattccacatggctgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatgctctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 74 - DNA, 7270 bp, Artificial Sequence
  Description of Artificial Sequence Synthetic polynucleotide
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagatggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgctggctacaggcagtggcgcaccactggcagacaataacgagggcgccgacggagtgggtaattcctcgggaaattggcattgcgattccacatggctgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatgctctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 75 - DNA, 3860 bp, Unknown
  Description of Unknown enterokinase sequence
agatttgttgtttgacaaaactttgaaaactggagagtttctgctcttcaactgctgcaagcttctgtgctcttccagagtccttagggtagcaaaccttcaaaaaccaaaaatggggtcaaagcgaagtgtaccatcaaggcaccgttctctcaccacctatgaagtcatgtttgccgttctctttgtcatattggtggcgctctgtgctggattaattgccgtgtcctggctgtcaatccagggatcagtaaaagatgcagcatttggaaaaagtcatgaagccagagggacattgaaaataatatccggagctacttataatcctcatttgcaagacaaactctcagtggacttcaaagttcttgcttttgacattcagcaaatgatagatgatatctttcaatcaagtaatctgaaaaatgaatataaaaactcaagagttttacaatttgaaaatggcagcattatagtcatatttgaccttctctttgaccagtgggtgtcagataaaaatgtaaaagaagaactgattcaaggcattgaagcaaataaatccagccaactggtcactttccacattgacttgaacagcattgatatcacagcctctttggagaatttctctacgataagtcctgcaacaacgtcagaaaagctaacaaccagcattcctctggcaaccccaggaaatgtctcaatagagtgcccacctgattcaaggctgtgtgctgatgctctaaagtgcatagcaattgatttattttgtgatggagaattaaactgtccagatggctctgatgaagacaataaaacttgtgccacagcttgtgatggaagatttttgttgactggatcttctgggtcctttgaggctctgcattatcccaagccttctaataatacaagcgctgtttgtcggtggattatacgtgtaaaccaaggactttccattcaactgaacttcgattattttaatacatattatgcagatgtattaaatatttatgaaggaatgggttcaagcaagattttaagagcttctctctggtcaaataatcctggcataattaggattttttccaatcaagttactgccacttttcttatacagtctgatgaaagtgattatattggcttcaaagtaacatacactgcatttaacagcaaagagcttaataattatgagaaaatcaactgtaattttgaagatggcttctgtttctggatccaggatctaaatgatgacaatgagtgggaaaggactcagggaagcacctttcctccatctactggaccaacttttgaccacacttttggcaatgagtcaggattttacatttccaccccaactggaccaggaggaagacgagaaagagtaggacttttaactctccctttagatcccactcctgaacaagcctgccttagtttctggtattatatgtatggtgaaaatgtttacaaactaagcattaatatcagcagtgaccaaaacatggagaagacaattttccaaaaagaaggaaattatggacaaaattggaactatggacaagtaacattaaatgaaacagtggaatttaaggtttctttctatgggtttaaaaaccagatcctgagtgatatagcattggatgacattagcctaacatatgggatttgtaatgtgagtgtctatccagaaccaactttagtcccaactcctccaccagaacttcccacggactgtggagggcctcatgacctgtgggagccaaatacaacattcacgtctataaacttcccaaacagctaccctaatcaggctttctgtatttggaatttaaatgcacaaaagggaaaaaatattcagctccactttcaagaatttgacctggaaaatattgcagatgtagttgaaatcagagatggtgaaggagatgattccttgttcttagctgtgtacacaggccctggtccagtaaacgatgtgttctcaaccaccaaccgaatgactgtgctttttatcactgataatatgctggcaaaacagggatttaaagcaaatttcactactggctatggcttggggattccagaaccctgcaaggaagacaattttcagtgcaaggatggggagtgtattccgctggtgaatctctgtgacggttttccacactgtaaggatggctcagatgaagcacactgtgtgcgtctcttcaatggcacgacagacagcagtggtttggtgcagttcaggatccaaagcatatggcatgtagcctgtgccgagaactggacaacccagatctcagatgatgtgtgtcagctgctgggactagggactggaaactcatccgtgccaaccttttctactggaggtggaccatatgtaaatttaaacacagcacctaatggcagcttaatactaacgccaagccaacagtgcttagaggattcactgattctgctacaatgtaactacaaatcatgtgggaaaaaactggtgactcaagaagttagcccgaagattgtcggaggaagtgactccagagaaggagcctggccttgggtcgttgctctgtatttcgacgatcaacaggtctgcggagcttctctggtgagcagggattggctggtgtcggccgcccactgcgtgtacgggagaaatatggagccgtctaagtggaaagcagtgctaggcctgcatatggcatcaaatctgacttctcctcagatagaaactaggttgattgaccaaattgtcataaacccacactacaataaacggagaaagaacaatgacattgccatgatgcatcttgaaatgaaagtgaactacacagattatatacagcctatttgtttaccagaagaaaatcaagtttttcccccaggaagaatttgttctattgctggctggggggcacttatatatcaaggttctactgcagacgtactgcaagaagctgacgttccccttctatcaaatgagaaatgtcaacaacagatgccagaatataacattacggaaaatatggtgtgtgcaggctatgaagcaggaggggtagattcttgtcagggggattcaggcggaccactcatgtgccaagaaaacaacagatggctcctggctggcgtgacgtcatttggatatcaatgtgcactgcctaatcgcccaggggtgtatgcccgggtcccaaggttcacagagtggatacaaagttttctacattagagtgtttccagaaacaaagatgaaaatcaggcagttttcccatttcactttaagaagcatggaaattgagagttaaaaaaataataatttataaaagtcttgattcttacctaaggcactgaaatgctacagaaaaaaaaaagcaaaaactaatctttacaatacaaagtaactataaaataataaattctgattttattgtcaacagttactctttcacagacatcattatttcctttgttcttaatcattatttttatcgtattcttatttaaagaaattatattttaaatcatgtaatataatgtttaagcaaagttaggaagagacatgaaataaacttttacacaaagtagggtattgtttgaaatagattgttataagttatctaattccaggataggtcactattatcagcatctcaatcattttgctgtttttctatccaaatgcattttcaatccatcttgagcacatccttaatattttccccataataaaatatatttattgtaagctcatgtcacaagcctggactaaactgattgtacaatcctttcaaataagctagttaaacagaaaactagcacaagtctatatattgcccttgcatcaaataaagctaaaataattaacattg

SEQ ID NO: 76 - protein, 1035 aa, Unknown
  Description of Unknown enterokinase sequence
MGSKRSVPSRHRSLTTYEVMFAVLFVILVALCAGLIAVSWLSIQGSVKDAAFGKSHEARGTLKIISGATYNPHLQDKLSVDFKVLAFDIQQMIDDIFQSSNLKNEYKNSRVLQFENGSIIVIFDLLFDQWVSDKNVKEELIQGIEANKSSQLVTFHIDLNSIDITASLENFSTISPATTSEKLTTSIPLATPGNVSIECPPDSRLCADALKCIAIDLFCDGELNCPDGSDEDNKTCATACDGRFLLTGSSGSFEALHYPKPSNNTSAVCRWIIRVNQGLSIQLNFDYFNTYYADVLNIYEGMGSSKILRASLWSNNPGIIRIFSNQVTATFLIQSDESDYIGFKVTYTAFNSKELNNYEKINCNFEDGFCFWIQDLNDDNEWERTQGSTFPPSTGPTFDHTFGNESGFYISTPTGPGGRRERVGLLTLPLDPTPEQACLSFWYYMYGENVYKLSINISSDQNMEKTIFQKEGNYGQNWNYGQVTLNETVEFKVSFYGFKNQILSDIALDDISLTYGICNVSVYPEPTLVPTPPPELPTDCGGPHDLWEPNTTFTSINFPNSYPNQAFCIWNLNAQKGKNIQLHFQEFDLENIADVVEIRDGEGDDSLFLAVYTGPGPVNDVFSTTNRMTVLFITDNMLAKQGFKANFTTGYGLGIPEPCKEDNFQCKDGECIPLVNLCDGFPHCKDGSDEAHCVRLFNGTTDSSGLVQFRIQSIWHVACAENWTTQISDDVCQLLGLGTGNSSVPTFSTGGGPYVNLNTAPNGSLILTPSQQCLEDSLILLQCNYKSCGKKLVTQEVSPKIVGGSDSREGAWPWVVALYFDDQQVCGASLVSRDWLVSAAHCVYGRNMEPSKWKAVLGLHMASNLTSPQIETRLIDQIVINPHYNKRRKNNDIAMMHLEMKVNYTDYIQPICLPEENQVFPPGRICSIAGWGALIYQGSTADVLQEADVPLLSNEKCQQQMPEYNITENMVCAGYEAGGVDSCQGDSGGPLMCQENNRWLLAGVTSFGYQCALPNRPGVYARVPRFTEWIQSFLH

SEQ ID NO: 77 - DNA, 7271 bp, Artificial Sequence
  Description of Artificial Sequence Synthetic polynucleotide
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatacaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagaaggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaataccatggctacaggcagtggcgcaccaatggcagacaataacgagggtgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatgctctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 78 - DNA, 6957 bp, Artificial Sequence
  Description of Artificial Sequence Synthetic polynucleotide
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctctagaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcatcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 79 - DNA, 4718 bp, Adeno-associated virus 1
ttgcccactccctctctgcgcgctcgctcgctcggtggggcctgcggaccaaaggtccgcagacggcagagctctgctctgccggccccaccgagcgagcgagcgcgcagagagggagtgggcaactccatcactaggggtaatcgcgaagcgcctcccacgctgccgcgtcagcgctgacgtaaattacgtcataggggagtggtcctgtattagctgtcacgtgagtgcttttgcgacattttgcgacaccacgtggccatttagggtatatatggccgagtgagcgagcaggatctccattttgaccgcgaaatttgaacgagcagcagccatgccgggcttctacgagatcgtgatcaaggtgccgagcgacctggacgagcacctgccgggcatttctgactcgtttgtgagctgggtggccgagaaggaatgggagctgcccccggattctgacatggatctgaatctgattgagcaggcacccctgaccgtggccgagaagctgcagcgcgacttcctggtccaatggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcctgtttgaacctggccgagcgcaaacggctcgtggcgcagcacctgacccacgtcagccagacccaggagcagaacaaggagaatctgaaccccaattctgacgcgcctgtcatccggtcaaaaacctccgcgcgctacatggagctggtcgggtggctggtggaccggggcatcacctccgagaagcagtggatccaggaggaccaggcctcgtacatctccttcaacgccgcttccaactcgcggtcccagatcaaggccgctctggacaatgccggcaagatcatggcgctgaccaaatccgcgcccgactacctggtaggccccgctccgcccgcggacattaaaaccaaccgcatctaccgcatcctggagctgaacggctacgaacctgcctacgccggctccgtctttctcggctgggcccagaaaaggttcgggaagcgcaacaccatctggctgtttgggccggccaccacgggcaagaccaacatcgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaagacatgcgagagaatgaatcagaatttcaacatttgcttcacgcacgggacgagagactgttcagagtgcttccccggcgtgtcagaatctcaaccggtcgtcagaaagaggacgtatcggaaactctgtgccattcatcatctgctggggcgggctcccgagattgcttgctcggcctgcgatctggtcaacgtggacctggatgactgtgtttctgagcaataaatgacttaaaccaggtatggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaattacgtgttaatcaataaaccggttgattcgtttcagttgaactttggtctcctgtccttcttatcttatcggttaccatggttatagcttacacattaactgcttggttgcgcttcgcgataaaagacttacgtcatcgggttacccctagtgatggagttgcccactccctctctgcgcgctcgctcgctcggtggggcctgcggaccaaaggtccgcagacggcagagctctgctctgccggccccaccgagcgagcgagcgcgcagagagggagtgggcaa

SEQ ID NO: 80 - DNA, 1872 bp, Adeno-associated virus 1
atgccgggcttctacgagatcgtgatcaaggtgccgagcgacctggacgagcacctgccgggcatttctgactcgtttgtgagctgggtggccgagaaggaatgggagctgcccccggattctgacatggatctgaatctgattgagcaggcacccctgaccgtggccgagaagctgcagcgcgacttcctggtccaatggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcctgtttgaacctggccgagcgcaaacggctcgtggcgcagcacctgacccacgtcagccagacccaggagcagaacaaggagaatctgaaccccaattctgacgcgcctgtcatccggtcaaaaacctccgcgcgctacatggagctggtcgggtggctggtggaccggggcatcacctccgagaagcagtggatccaggaggaccaggcctcgtacatctccttcaacgccgcttccaactcgcggtcccagatcaaggccgctctggacaatgccggcaagatcatggcgctgaccaaatccgcgcccgactacctggtaggccccgctccgcccgcggacattaaaaccaaccgcatctaccgcatcctggagctgaacggctacgaacctgcctacgccggctccgtctttctcggctgggcccagaaaaggttcgggaagcgcaacaccatctggctgtttgggccggccaccacgggcaagaccaacatcgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaagacatgcgagagaatgaatcagaatttcaacatttgcttcacgcacgggacgagagactgttcagagtgcttccccggcgtgtcagaatctcaaccggtcgtcagaaagaggacgtatcggaaactctgtgccattcatcatctgctggggcgggctcccgagattgcttgctcggcctgcgatctggtcaacgtggacctggatgactgtgtttctgagcaataa

SEQ ID NO: 81 - DNA, 2211 bp, Adeno-associated virus 1
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 82 - DNA, 8376 bp, Adeno-associated virus 2; identical to SEQ ID NO: 70

SEQ ID NO: 83 - DNA, 1882 bp, Adeno-associated virus 2
acgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggt

SEQ ID NO: 84 - DNA, 2208 bp, Adeno-associated virus 2
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 85 - DNA, 4726 bp, Adeno-associated virus 3
ttggccactccctctatgcgcactcgctcgctcggtggggcctggcgaccaaaggtcgccagacggacgtgctttgcacgtccggccccaccgagcgagcgagtgcgcatagagggagtggccaactccatcactagaggtatggcagtgacgtaacgcgaagcgcgcgaagcgagaccacgcctaccagctgcgtcagcagtcaggtgacccttttgcgacagtttgcgacaccacgtggccgctgagggtatatattctcgagtgagcgaaccaggagctccattttgaccgcgaaatttgaacgagcagcagccatgccggggttctacgagattgtcctgaaggtcccgagtgacctggacgagcgcctgccgggcatttctaactcgtttgttaactgggtggccgagaaggaatgggacgtgccgccggattctgacatggatccgaatctgattgagcaggcacccctgaccgtggccgaaaagcttcagcgcgagttcctggtggagtggcgccgcgtgagtaaggccccggaggccctcttttttgtccagttcgaaaagggggagacctacttccacctgcacgtgctgattgagaccatcggggtcaaatccatggtggtcggccgctacgtgagccagattaaagagaagctggtgacccgcatctaccgcggggtcgagccgcagcttccgaactggttcgcggtgaccaaaacgcgaaatggcgccgggggcgggaacaaggtggtggacgactgctacatccccaactacctgctccccaagacccagcccgagctccagtgggcgtggactaacatggaccagtatttaagcgcctgtttgaatctcgcggagcgtaaacggctggtggcgcagcatctgacgcacgtgtcgcagacgcaggagcagaacaaagagaatcagaaccccaattctgacgcgccggtcatcaggtcaaaaacctcagccaggtacatggagctggtcgggtggctggtggaccgcgggatcacgtcagaaaagcaatggattcaggaggaccaggcctcgtacatctccttcaacgccgcctccaactcgcggtcccagatcaaggccgcgctggacaatgcctccaagatcatgagcctgacaaagacggctccggactacctggtgggcagcaacccgccggaggacattaccaaaaatcggatctaccaaatcctggagctgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtaaactggaccaatgagaactttcccttcaacgattgcgtcgacaagatggtgatctggtgggaggagggcaagatgacggccaaggtcgtggagagcgccaaggccattctgggcggaagcaaggtgcgcgtggaccaaaagtgcaagtcatcggcccagatcgaacccactcccgtgatcgtcacctccaacaccaacatgtgcgccgtgattgacgggaacagcaccaccttcgagcatcagcagccgctgcaggaccggatgtttgaatttgaacttacccgccgtttggaccatgactttgggaaggtcaccaaacaggaagtaaaggactttttccggtgggcttccgatcacgtgactgacgtggctcatgagttctacgtcagaaagggtggagctaagaaacgccccgcctccaatgacgcggatgtaagcgagccaaaacgggagtgcacgtcacttgcgcagccgacaacgtcagacgcggaagcaccggcggactacgcggacaggtaccaaaacaaatgttctcgtcacgtgggcatgaatctgatgctttttccctgtaaaacatgcgagagaatgaatcaaatttccaatgtctgttttacgcatggtcaaagagactgtggggaatgcttccctggaatgtcagaatctcaacccgtttctgtcgtcaaaaagaagacttatcagaaactgtgtccaattcatcatatcctgggaagggcacccgagattgcctgttcggcctgcgatttggccaatgtggacttggatgactgtgtttctgagcaataaatgacttaaaccaggtatggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagggggctgtagatcagtctcctcaggaaccggactcatcatctggtgttggcaaatcgggcaaacagcctgccagaaaaagactaaatttcggtcagactggagactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactggaactgtcaatcatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtgaatcctggttaatcaataaaccgtttaattcgtttcagttgaactttggctcttgtgcacttctttatctttatcttgtttccatggctactgcgtagataagcagcggcctgcggcgcttgcgcttcgcggtttacaactgctggttaatatttaactctcgccatacctctagtgatggagttggccactccctctatgcgcactcgctcgctcggtggggcctggcgaccaaaggtcgccagacggacgtgctttgcacgtccggccccaccgagcgagcgagtgcgcatagagggagtggccaa

SEQ ID NO: 86 - DNA, 1812 bp, Adeno-associated virus 3
atgccggggttctacgagattgtcctgaaggtcccgagtgacctggacgagcgcctgccgggcatttctaactcgtttgttaactgggtggccgagaaggaatgggacgtgccgccggattctgacatggatccgaatctgattgagcaggcacccctgaccgtggccgaaaagcttcagcgcgagttcctggtggagtggcgccgcgtgagtaaggccccggaggccctcttttttgtccagttcgaaaagggggagacctacttccacctgcacgtgctgattgagaccatcggggtcaaatccatggtggtcggccgctacgtgagccagattaaagagaagctggtgacccgcatctaccgcggggtcgagccgcagcttccgaactggttcgcggtgaccaaaacgcgaaatggcgccgggggcgggaacaaggtggtggacgactgctacatccccaactacctgctccccaagacccagcccgagctccagtgggcgtggactaacatggaccagtatttaagcgcctgtttgaatctcgcggagcgtaaacggctggtggcgcagcatctgacgcacgtgtcgcagacgcaggagcagaacaaagagaatcagaaccccaattctgacgcgccggtcatcaggtcaaaaacctcagccaggtacatggagctggtcgggtggctggtggaccgcgggatcacgtcagaaaagcaatggattcaggaggaccaggcctcgtacatctccttcaacgccgcctccaactcgcggtcccagatcaaggccgcgctggacaatgcctccaagatcatgagcctgacaaagacggctccggactacctggtgggcagcaacccgccggaggacattaccaaaaatcggatctaccaaatcctggagctgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtaaactggaccaatgagaactttcccttcaacgattgcgtcgacaagatggtgatctggtgggaggagggcaagatgacggccaaggtcgtggagagcgccaaggccattctgggcggaagcaaggtgcgcgtggaccaaaagtgcaagtcatcggcccagatcgaacccactcccgtgatcgtcacctccaacaccaacatgtgcgccgtgattgacgggaacagcaccaccttcgagcatcagcagccgctgcaggaccggatgtttgaatttgaacttacccgccgtttggaccatgactttgggaaggtcaccaaacaggaagtaaaggactttttccggtgggcttccgatcacgtgactgacgtggctcatgagttctacgtcagaaagggtggagctaagaaacgccccgcctccaatgacgcggatgtaagcgagccaaaacgggagtgcacgtcacttgcgcagccgacaacgtcagacgcggaagcaccggcggactacgcggacaggtaccaaaacaaatgttctcgtcacgtgggcatgaatctgatgctttttccctgtaaaacatgcgagagaatgaatcaaatttccaatgtctgttttacgcatggtcaaagagactgtggggaatgcttccctggaatgtcagaatctcaacccgtttctgtcgtcaaaaagaagacttatcagaaactgtgtccaattcatcatatcctgggaagggcacccgag

SEQ ID NO: 87 - DNA, 2211 bp, Adeno-associated virus 3
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagggggctgtagatcagtctcctcaggaaccggactcatcatctggtgttggcaaatcgggcaaacagcctgccagaaaaagactaaatttcggtcagactggagactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactggaactgtcaatcatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtga

SEQ ID NO: 88 - DNA, 4767 bp, Adeno-associated virus 4
ttggccactccctctatgcgcgctcgctcactcactcggccctggagaccaaaggtctccagactgccggcctctggccggcagggccgagtgagtgagcgagcgcgcatagagggagtggccaactccatcatctaggtttgcccactgacgtcaatgtgacgtcctagggttagggaggtccctgtattagcagtcacgtgagtgtcgtatttcgcggagcgtagcggagcgcataccaagctgccacgtcacagccacgtggtccgtttgcgacagtttgcgacaccatgtggtcaggagggtatataaccgcgagtgagccagcgaggagctccattttgcccgcgaattttgaacgagcagcagccatgccggggttctacgagatcgtgctgaaggtgcccagcgacctggacgagcacctgcccggcatttctgactcttttgtgagctgggtggccgagaaggaatgggagctgccgccggattctgacatggacttgaatctgattgagcaggcacccctgaccgtggccgaaaagctgcaacgcgagttcctggtcgagtggcgccgcgtgagtaaggccccggaggccctcttctttgtccagttcgagaagggggacagctacttccacctgcacatcctggtggagaccgtgggcgtcaaatccatggtggtgggccgctacgtgagccagattaaagagaagctggtgacccgcatctaccgcggggtcgagccgcagcttccgaactggttcgcggtgaccaagacgcgtaatggcgccggaggcgggaacaaggtggtggacgactgctacatccccaactacctgctccccaagacccagcccgagctccagtgggcgtggactaacatggaccagtatataagcgcctgtttgaatctcgcggagcgtaaacggctggtggcgcagcatctgacgcacgtgtcgcagacgcaggagcagaacaaggaaaaccagaaccccaattctgacgcgccggtcatcaggtcaaaaacctccgccaggtacatggagctggtcgggtggctggtggaccgcgggatcacgtcagaaaagcaatggatccaggaggaccaggcgtcctacatctccttcaacgccgcctccaactcgcggtcacaaatcaaggccgcgctggacaatgcctccaaaatcatgagcctgacaaagacggctccggactacctggtgggccagaacccgccggaggacatttccagcaaccgcatctaccgaatcctcgagatgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtgaactggaccaatgagaactttccgttcaacgattgcgtcgacaagatggtgatctggtgggaggagggcaagatgacggccaaggtcgtagagagcgccaaggccatcctgggcggaagcaaggtgcgcgtggaccaaaagtgcaagtcatcggcccagatcgacccaactcccgtgatcgtcacctccaacaccaacatgtgcgcggtcatcgacggaaactcgaccaccttcgagcaccaacaaccactccaggaccggatgttcaagttcgagctcaccaagcgcctggagcacgactttggcaaggtcaccaagcaggaagtcaaagactttttccggtgggcgtcagatcacgtgaccgaggtgactcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacaggtaccaaaacaaatgttctcgtcacgtgggtatgaatctgatgctttttccctgccggcaatgcgagagaatgaatcagaatgtggacatttgcttcacgcacggggtcatggactgtgccgagtgcttccccgtgtcagaatctcaacccgtgtctgtcgtcagaaagcggacgtatcagaaactgtgtccgattcatcacatcatggggagggcgcccgaggtggcctgctcggcctgcgaactggccaatgtggacttggatgactgtgacatggaacaataaatgactcaaaccagatatgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaataacctgttaatcaataaaccggtttattcgtttcagttgaactttggtctccgtgtccttcttatcttatctcgtttccatggctactgcgtacataagcagcggcctgcggcgcttgcgcttcgcggtttacaactgccggttaatcagtaacttctggcaaaccagatgatggagttggccacattagctatgcgcgctcgctcactcactcggccctggagaccaaaggtctccagactgccggcctctggccggcagggccgagtgagtgagcgagcgcgcatagagggagtggccaa

SEQ ID NO: 89 - DNA, 1872 bp, Adeno-associated virus 4
atgccggggttctacgagatcgtgctgaaggtgcccagcgacctggacgagcacctgcccggcatttctgactcttttgtgagctgggtggccgagaaggaatgggagctgccgccggattctgacatggacttgaatctgattgagcaggcacccctgaccgtggccgaaaagctgcaacgcgagttcctggtcgagtggcgccgcgtgagtaaggccccggaggccctcttctttgtccagttcgagaagggggacagctacttccacctgcacatcctggtggagaccgtgggcgtcaaatccatggtggtgggccgctacgtgagccagattaaagagaagctggtgacccgcatctaccgcggggtcgagccgcagcttccgaactggttcgcggtgaccaagacgcgtaatggcgccggaggcgggaacaaggtggtggacgactgctacatccccaactacctgctccccaagacccagcccgagctccagtgggcgtggactaacatggaccagtatataagcgcctgtttgaatctcgcggagcgtaaacggctggtggcgcagcatctgacgcacgtgtcgcagacgcaggagcagaacaaggaaaaccagaaccccaattctgacgcgccggtcatcaggtcaaaaacctccgccaggtacatggagctggtcgggtggctggtggaccgcgggatcacgtcagaaaagcaatggatccaggaggaccaggcgtcctacatctccttcaacgccgcctccaactcgcggtcacaaatcaaggccgcgctggacaatgcctccaaaatcatgagcctgacaaagacggctccggactacctggtgggccagaacccgccggaggacatttccagcaaccgcatctaccgaatcctcgagatgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtgaactggaccaatgagaactttccgttcaacgattgcgtcgacaagatggtgatctggtgggaggagggcaagatgacggccaaggtcgtagagagcgccaaggccatcctgggcggaagcaaggtgcgcgtggaccaaaagtgcaagtcatcggcccagatcgacccaactcccgtgatcgtcacctccaacaccaacatgtgcgcggtcatcgacggaaactcgaccaccttcgagcaccaacaaccactccaggaccggatgttcaagttcgagctcaccaagcgcctggagcacgactttggcaaggtcaccaagcaggaagtcaaagactttttccggtgggcgtcagatcacgtgaccgaggtgactcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacaggtaccaaaacaaatgttctcgtcacgtgggtatgaatctgatgctttttccctgccggcaatgcgagagaatgaatcagaatgtggacatttgcttcacgcacggggtcatggactgtgccgagtgcttccccgtgtcagaatctcaacccgtgtctgtcgtcagaaagcggacgtatcagaaactgtgtccgattcatcacatcatggggagggcgcccgaggtggcctgctcggcctgcgaactggccaatgtggacttggatgactgtgacatggaacaataa

SEQ ID NO: 90 - DNA, 2205 bp, Adeno-associated virus 4
atgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaa

SEQ ID NO: 91 - DNA, 4642 bp, Adeno-associated virus 5
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgtaagcagtgatgtcataatgatgtaatgcttattgtcacgcgatagttaatgattaacagtcatgtgatgtgttttatccaataggaagaaagcgcgcgtatgagttctcgcgagacttccggggtataaaagaccgagtgaacgagcccgccgccattctttgctctggactgctagaggaccctcgctgccatggctaccttctatgaagtcattgttcgcgtcccatttgacgtggaggaacatctgcctggaatttctgacagctttgtggactgggtaactggtcaaatttgggagctgcctccagagtcagatttaaatttgactctggttgaacagcctcagttgacggtggctgatagaattcgccgcgtgttcctgtacgagtggaacaaattttccaagcaggagtccaaattctttgtgcagtttgaaaagggatctgaatattttcatctgcacacgcttgtggagacctccggcatctcttccatggtcctcggccgctacgtgagtcagattcgcgcccagctggtgaaagtggtcttccagggaattgaaccccagatcaacgactgggtcgccatcaccaaggtaaagaagggcggagccaataaggtggtggattctgggtatattcccgcctacctgctgccgaaggtccaaccggagcttcagtgggcgtggacaaacctggacgagtataaattggccgccctgaatctggaggagcgcaaacggctcgtcgcgcagtttctggcagaatcctcgcagcgctcgcaggaggcggcttcgcagcgtgagttctcggctgacccggtcatcaaaagcaagacttcccagaaatacatggcgctcgtcaactggctcgtggagcacggcatcacttccgagaagcagtggatccaggaaaatcaggagagctacctctccttcaactccaccggcaactctcggagccagatcaaggccgcgctcgacaacgcgaccaaaattatgagtctgacaaaaagcgcggtggactacctcgtggggagctccgttcccgaggacatttcaaaaaacagaatctggcaaatttttgagatgaatggctacgacccggcctacgcgggatccatcctctacggctggtgtcagcgctccttcaacaagaggaacaccgtctggctctacggacccgccacgaccggcaagaccaacatcgcggaggccatcgcccacactgtgcccttttacggctgcgtgaactggaccaatgaaaactttccctttaatgactgtgtggacaaaatgctcatttggtgggaggagggaaagatgaccaacaaggtggttgaatccgccaaggccatcctggggggctcaaaggtgcgggtcgatcagaaatgtaaatcctctgttcaaattgattctacccctgtcattgtaacttccaatacaaacatgtgtgtggtggtggatgggaattccacgacctttgaacaccagcagccgctggaggaccgcatgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctcaattggaattcaaggtatgattgcaaatgtgactatcatgctcaatttgacaacatttctaacaaatgtgatgaatgtgaatatttgaatcggggcaaaaatggatgtatctgtcacaatgtaactcactgtcaaatttgtcatgggattcccccctgggaaaaggaaaacttgtcagattttggggattttgacgatgccaataaagaacagtaaataaagcgagtagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaacccattcatgtcgcataccctcaataaaccgtgtattcgtgtcagtaaaatactgcctcttgtggtcattcaatgaataacagcttacaacatctacaaaacctccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacaggggggagag

SEQ ID NO: 92 - DNA, 1833 bp, Adeno-associated virus 5
atggctaccttctatgaagtcattgttcgcgtcccatttgacgtggaggaacatctgcctggaatttctgacagctttgtggactgggtaactggtcaaatttgggagctgcctccagagtcagatttaaatttgactctggttgaacagcctcagttgacggtggctgatagaattcgccgcgtgttcctgtacgagtggaacaaattttccaagcaggagtccaaattctttgtgcagtttgaaaagggatctgaatattttcatctgcacacgcttgtggagacctccggcatctcttccatggtcctcggccgctacgtgagtcagattcgcgcccagctggtgaaagtggtcttccagggaattgaaccccagatcaacgactgggtcgccatcaccaaggtaaagaagggcggagccaataaggtggtggattctgggtatattcccgcctacctgctgccgaaggtccaaccggagcttcagtgggcgtggacaaacctggacgagtataaattggccgccctgaatctggaggagcgcaaacggctcgtcgcgcagtttctggcagaatcctcgcagcgctcgcaggaggcggcttcgcagcgtgagttctcggctgacccggtcatcaaaagcaagacttcccagaaatacatggcgctcgtcaactggctcgtggagcacggcatcacttccgagaagcagtggatccaggaaaatcaggagagctacctctccttcaactccaccggcaactctcggagccagatcaaggccgcgctcgacaacgcgaccaaaattatgagtctgacaaaaagcgcggtggactacctcgtggggagctccgttcccgaggacatttcaaaaaacagaatctggcaaatttttgagatgaatggctacgacccggcctacgcgggatccatcctctacggctggtgtcagcgctccttcaacaagaggaacaccgtctggctctacggacccgccacgaccggcaagaccaacatcgcggaggccatcgcccacactgtgcccttttacggctgcgtgaactggaccaatgaaaactttccctttaatgactgtgtggacaaaatgctcatttggtgggaggagggaaagatgaccaacaaggtggttgaatccgccaaggccatcctggggggctcaaaggtgcgggtcgatcagaaatgtaaatcctctgttcaaattgattctacccctgtcattgtaacttccaatacaaacatgtgtgtggtggtggatgggaattccacgacctttgaacaccagcagccgctggaggaccgcatgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctcaattggaattcaaggtatgattgcaaatgtgactatcatgctcaatttgacaacatttctaacaaatgtgatgaatgtgaatatttgaatcggggcaaaaatggatgtatctgtcacaatgtaactcactgtcaaatttgtcatgggattcccccctgggaaaaggaaaacttgtcagattttggggattttgacgatgccaataaagaacagtaa

SEQ ID NO: 93 - DNA, 2175 bp, Adeno-associated virus 5
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 94 - DNA, 4683 bp, Adeno-associated virus 6
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagcgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgacttcctggtccagtggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcgtgtttaaacctggccgagcgcaaacggctcgtggcgcacgacctgacccacgtcagccagacccaggagcagaacaaggagaatctgaaccccaattctgacgcgcctgtcatccggtcaaaaacctccgcacgctacatggagctggtcgggtggctggtggaccggggcatcacctccgagaagcagtggatccaggaggaccaggcctcgtacatctccttcaacgccgcctccaactcgcggtcccagatcaaggccgctctggacaatgccggcaagatcatggcgctgaccaaatccgcgcccgactacctggtaggccccgctccgcccgccgacattaaaaccaaccgcatttaccgcatcctggagctgaacggctacgaccctgcctacgccggctccgtctttctcggctgggcccagaaaaggttcggaaaacgcaacaccatctggctgtttgggccggccaccacgggcaagaccaacatcgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgatcccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaagagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacatgcgagagaatgaatcagaatttcaacatttgcttcacgcacgggaccagagactgttcagaatgtttccccggcgtgtcagaatctcaaccggtcgtcagaaagaggacgtatcggaaactctgtgccattcatcatctgctggggcgggctcccgagattgcttgctcggcctgcgatctggtcaacgtggatctggatgactgtgtttctgagcaataaatgacttaaaccaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgtgtgttaatcaataaaccggttaattcgtgtcagttgaactttggtctcatgtcgttattatcttatctggtcaccatagcaaccggttacacattaactgcttagttgcgcttcgcgaatacccctagtgatggagttgcccactccctctatgcgcgctcgctcgctcggtggggccggcagagcagagctctgccgtctgcggacctttggtccgcaggccccaccgagcgagcgagcgcgcatagagggagtgggcaa

SEQ ID NO: 95 - DNA, 1872 bp, Adeno-associated virus 6
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgacttcctggtccagtggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcgtgtttaaacctggccgagcgcaaacggctcgtggcgcacgacctgacccacgtcagccagacccaggagcagaacaaggagaatctgaaccccaattctgacgcgcctgtcatccggtcaaaaacctccgcacgctacatggagctggtcgggtggctggtggaccggggcatcacctccgagaagcagtggatccaggaggaccaggcctcgtacatctccttcaacgccgcctccaactcgcggtcccagatcaaggccgctctggacaatgccggcaagatcatggcgctgaccaaatccgcgcccgactacctggtaggccccgctccgcccgccgacattaaaaccaaccgcatttaccgcatcctggagctgaacggctacgaccctgcctacgccggctccgtctttctcggctgggcccagaaaaggttcggaaaacgcaacaccatctggctgtttgggccggccaccacgggcaagaccaacatcgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgatcccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaagagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacatgcgagagaatgaatcagaatttcaacatttgcttcacgcacgggaccagagactgttcagaatgtttccccggcgtgtcagaatctcaaccggtcgtcagaaagaggacgtatcggaaactctgtgccattcatcatctgctggggcgggctcccgagattgcttgctcggcctgcgatctggtcaacgtggatctggatgactgtgtttctgagcaataa

SEQ ID NO: 96 - DNA, 2211 bp, Adeno-associated virus 6
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 97 - DNA, 4721 bp, Adeno-associated virus 7
ttggccactccctctatgcgcgctcgctcgctcggtggggcctgcggaccaaaggtccgcagacggcagagctctgctctgccggccccaccgagcgagcgagcgcgcatagagggagtggccaactccatcactaggggtaccgcgaagcgcctcccacgctgccgcgtcagcgctgacgtaaatcacgtcataggggagtggtcctgtattagctgtcacgtgagtgcttttgcgacattttgcgacaccacgtggccatttgaggtatatatggccgagtgagcgagcaggatctccattttgaccgcgaaatttgaacgagcagcagccatgccgggtttctacgagatcgtgatcaaggtgccgagcgacctggacgagcacctgccgggcatttctgactcgtttgtgaactgggtggccgagaaggaatgggagctgcccccggattctgacatggatctgaatctgatcgagcaggcacccctgaccgtggccgagaagctgcagcgcgacttcctggtccaatggcgccgcgtgagtaaggccccggaggccctgttctttgttcagttcgagaagggcgagagctacttccaccttcacgttctggtggagaccacgggggtcaagtccatggtgctaggccgcttcctgagtcagattcgggagaagctggtccagaccatctaccgcggggtcgagcccacgctgcccaactggttcgcggtgaccaagacgcgtaatggcgccggcggggggaacaaggtggtggacgagtgctacatccccaactacctcctgcccaagacccagcccgagctgcagtgggcgtggactaacatggaggagtatataagcgcgtgtttgaacctggccgaacgcaaacggctcgtggcgcagcacctgacccacgtcagccagacgcaggagcagaacaaggagaatctgaaccccaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattaaaaccaaccgcatctaccgcatcctggagctgaacgggtacgatcctgcctacgccggctccgtctttctcggctgggcccagaaaaagttcgggaagcgcaacaccatctggctgtttgggcccgccaccaccggcaagaccaacattgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaaatttgaactcacccgccgtctggagcacgactttggcaaggtgacgaagcaggaagtcaaagagttcttccgctgggccagtgatcacgtgaccgaggtggcgcatgagttctacgtcagaaagggcggagccagcaaaagacccgcccccgatgacgcggatataagcgagcccaagcgggcctgcccctcagtcgcggatccatcgacgtcagacgcggaaggagctccggtggactttgccgacaggtaccaaaacaaatgttctcgtcacgcgggcatgattcagatgctgtttccctgcaaaacgtgcgagagaatgaatcagaatttcaacatttgcttcacacacggggtcagagactgtttagagtgtttccccggcgtgtcagaatctcaaccggtcgtcagaaaaaagacgtatcggaaactctgcgcgattcatcatctgctggggcgggcgcccgagattgcttgctcggcctgcgacctggtcaacgtggacctggacgactgcgtttctgagcaataaatgacttaaaccaggtatggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaattgcatgttaatcaataaaccggttgattcgtttcagttgaactttggtctcctgtgcttcttatcttatcggtttccatagcaactggttacacattaactgcttgggtgcgcttcacgataagaacactgacgtcaccgcggtacccctagtgatggagttggccactccctctatgcgcgctcgctcgctcggtggggcctgcggaccaaaggtccgcagacggcagagctctgctctgccggccccaccgagcgagcgagcgcgcatagagggagtggccaa

SEQ ID NO: 98 - DNA, 1872 bp, Adeno-associated virus 7
atgccgggtttctacgagatcgtgatcaaggtgccgagcgacctggacgagcacctgccgggcatttctgactcgtttgtgaactgggtggccgagaaggaatgggagctgcccccggattctgacatggatctgaatctgatcgagcaggcacccctgaccgtggccgagaagctgcagcgcgacttcctggtccaatggcgccgcgtgagtaaggccccggaggccctgttctttgttcagttcgagaagggcgagagctacttccaccttcacgttctggtggagaccacgggggtcaagtccatggtgctaggccgcttcctgagtcagattcgggagaagctggtccagaccatctaccgcggggtcgagcccacgctgcccaactggttcgcggtgaccaagacgcgtaatggcgccggcggggggaacaaggtggtggacgagtgctacatccccaactacctcctgcccaagacccagcccgagctgcagtgggcgtggactaacatggaggagtatataagcgcgtgtttgaacctggccgaacgcaaacggctcgtggcgcagcacctgacccacgtcagccagacgcaggagcagaacaaggagaatctgaaccccaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattaaaaccaaccgcatctaccgcatcctggagctgaacgggtacgatcctgcctacgccggctccgtctttctcggctgggcccagaaaaagttcgggaagcgcaacaccatctggctgtttgggcccgccaccaccggcaagaccaacattgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaaatttgaactcacccgccgtctggagcacgactttggcaaggtgacgaagcaggaagtcaaagagttcttccgctgggccagtgatcacgtgaccgaggtggcgcatgagttctacgtcagaaagggcggagccagcaaaagacccgcccccgatgacgcggatataagcgagcccaagcgggcctgcccctcagtcgcggatccatcgacgtcagacgcggaaggagctccggtggactttgccgacaggtaccaaaacaaatgttctcgtcacgcgggcatgattcagatgctgtttccctgcaaaacgtgcgagagaatgaatcagaatttcaacatttgcttcacacacggggtcagagactgtttagagtgtttccccggcgtgtcagaatctcaaccggtcgtcagaaaaaagacgtatcggaaactctgcgcgattcatcatctgctggggcgggcgcccgagattgcttgctcggcctgcgacctggtcaacgtggacctggacgactgcgtttctgagcaataa

SEQ ID NO: 99 - DNA, 2214 bp, Adeno-associated virus 7
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaa

SEQ ID NO: 100 - DNA, 4393 bp, Adeno-associated virus 8
cagagagggagtggccaactccatcactaggggtagcgcgaagcgcctcccacgctgccgcgtcagcgctgacgtaaattacgtcataggggagtggtcctgtattagctgtcacgtgagtgcttttgcggcattttgcgacaccacgtggccatttgaggtatatatggccgagtgagcgagcaggatctccattttgaccgcgaaatttgaacgagcagcagccatgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccaatggcgccgcgtgagtaaggccccggaggccctcttctttgttcagttcgagaagggcgagagctactttcacctgcacgttctggtcgagaccacgggggtcaagtccatggtgctaggccgcttcctgagtcagattcgggaaaagcttggtccagaccatctacccgcggggtcgagccccaccttgcccaactggttcgcggtgaccaaagacgcggtaatggcgccggcgggggggaacaaggtggtggacgagtgctacatccccaactacctcctgcccaagactcagcccgagctgcagtgggcgtggactaacatggaggagtatataagcgcgtgcttgaacctggccgagcgcaaacggctcgtggcgcagcacctgacccacgtcagccagacgcaggagcagaacaaggagaatctgaaccccaattctgacgcgcccgtgatcaggtcaaaaacctccgcgcgctatatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattacccagaaccgcatctaccgcatcctcgctctcaacggctacgaccctgcctacgccggctccgtctttctcggctgggctcagaaaaagttcgggaaacgcaacaccatctggctgtttggacccgccaccaccggcaagaccaacattgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagcctctccaggaccggatgtttaagttcgaactcacccgccgtctggagcacgactttggcaaggtgacaaagcaggaagtcaaagagttcttccgctgggccagtgatcacgtgaccgaggtggcgcatgagttttacgtcagaaagggcggagccagcaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacgtgcgagagaatgaatcagaatttcaacatttgcttcacacacggggtcagagactgctcagagtgtttccccggcgtgtcagaatctcaaccggtcgtcagaaagaggacgtatcggaaactctgtgcgattcatcatctgctggggcgggctcccgagattgcttgctcggcctgcgatctggtcaacgtggacctggatgactgtgtttctgagcaataaatgacttaaaccaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcg

SEQ ID NO: 101 - DNA, 1878 bp, Adeno-associated virus 8
atgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccaatggcgccgcgtgagtaaggccccggaggccctcttctttgttcagttcgagaagggcgagagctactttcacctgcacgttctggtcgagaccacgggggtcaagtccatggtgctaggccgcttcctgagtcagattcgggaaaagcttggtccagaccatctacccgcggggtcgagccccaccttgcccaactggttcgcggtgaccaaagacgcggtaatggcgccggcgggggggaacaaggtggtggacgagtgctacatccccaactacctcctgcccaagactcagcccgagctgcagtgggcgtggactaacatggaggagtatataagcgcgtgcttgaacctggccgagcgcaaacggctcgtggcgcagcacctgacccacgtcagccagacgcaggagcagaacaaggagaatctgaaccccaattctgacgcgcccgtgatcaggtcaaaaacctccgcgcgctatatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattacccagaaccgcatctaccgcatcctcgctctcaacggctacgaccctgcctacgccggctccgtctttctcggctgggctcagaaaaagttcgggaaacgcaacaccatctggctgtttggacccgccaccaccggcaagaccaacattgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagcctctccaggaccggatgtttaagttcgaactcacccgccgtctggagcacgactttggcaaggtgacaaagcaggaagtcaaagagttcttccgctgggccagtgatcacgtgaccgaggtggcgcatgagttttacgtcagaaagggcggagccagcaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacgtgcgagagaatgaatcagaatttcaacatttgcttcacacacggggtcagagactgctcagagtgtttccccggcgtgtcagaatctcaaccggtcgtcagaaagaggacgtatcggaaactctgtgcgattcatcatctgctggggcgggctcccgagattgcttgctcggcctgcgatctggtcaacgtggacctggatgactgtgtttctgagcaataa

SEQ ID NO: 102 - DNA, 2217 bp, Adeno-associated virus 8
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 103 - DNA, 6042 bp, Adeno-associated virus 9
gcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaaggagaaatgctggcatagagcagcactaaatgacaccactaaagaaacgatcagacagatctagaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 104 - DNA, 4102 bp, Adeno-associated virus 10
atgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccactggcgccgcgtgagtaaggccccggaggccctcttctttgttcagttcgagaagggcgagtcctactttcacctgcacgttctggtcgagaccacgggggtcaagtccatggtcctgggccgcttcctgagtcagatcagagacaggctggtgcagaccatctaccgcggggtagagcccacgctgcccaactggttcgcggtgaccaagacgcgaaatggcgccggcggggggaacaaggtggtggacgagtgctacatccccaactacctcctgcccaagacgcagcccgagctgcagtgggcgtggactaacatggaggagtatataagcgcgtgtctgaacctcgcggagcgtaaacggctcgtggcgcagcacctgacccacgtcagccagacgcaggagcagaacaaggagaatctgaacccgaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggaaagatcatggcgctgaccaaatccgcgcccgactacctggtaggcccgtccttacccgcggacattaaggccaaccgcatctaccgcatcctggagctcaacggctacgaccccgcctacgccggctccgtcttcctgggctgggcgcagaaaaagttcggtaaaaggaatacaatttggctgttcgggcccgccaccaccggcaagaccaacatcgcggaagccatcgcccacgccgtgcccttctacggctgcgtcaactggaccaatgagaactttcccttcaacgattgcgtcgacaagatggtgatctggtgggaggagggcaagatgaccgccaaggtcgtggagtccgccaaggccattctgggcggaagcaaggtgcgcgtcgaccaaaagtgcaagtcctcggcccagatcgaccccacgcccgtgatcgtcacctccaacaccaacatgtgcgccgtgatcgacgggaacagcaccaccttcgagcaccagcagcccctgcaggaccgcatgttcaagttcgagctcacccgccgtctggagcacgactttggcaaggtgaccaagcaggaagtcaaagagttcttccgctgggctcaggatcacgtgactgaggtgacgcatgagttctacgtcagaaagggcggagccaccaaaagacccgcccccagtgacgcggatataagcgagcccaagcgggcctgcccctcagttgcggagccatcgacgtcagacgcggaagcaccggtggactttgcggacaggtaccaaaacaaatgttctcgtcacgcgggcatgcttcagatgctgtttccctgcaagacatgcgagagaatgaatcagaatttcaacgtctgcttcacgcacggggtcagagactgctcagagtgcttccccggcgcgtcagaatctcaacctgtcgtcagaaaaaagacgtatcagaaactgtgcgcgattcatcatctgctggggcgggcacccgagattgcgtgttcggcctgcgatctcgtcaacgtggacttggatgactgtgtttctgagcaataaatgacttaaaccaggtatggctgctgacggttatcttccagattggctcgaggacaacctctctgagggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgttcccccgactcctccacgggcatcggcaagaaaggccagcagcccgctaaaaagagactgaactttgggcagactggcgagtcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatctccaacgggacatcgggaggaagcaccaacgacaacacctacttcggctacagcaccccctgggggtattttgacttcaacagattccactgccacttctcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggatgtcttcatgattccccagtacggctacctgacactgaacaatggaagtcaagccgtaggccgttcctccttctactgcctggaatattttccatctcaaatgctgcgaactggaaacaattttgaattcagctacaccttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggaagagactctctggtgaatcccggtgtcgccatggcaacccacaaggacgacgaggaacgcttcttcccgtcgagcggagtcctgatgtttggaaaacagggtgctggaagagacaatgtggactacagcagcgttatgctaacaagcgaagaagaaattaaaaccactaaccctgtagccacagaacaatacggcgtggtggctgacaacttgcagcaagccaatacagggcctattgtgggaaatgtcaacagccaaggagccttacctggcatggtctggcagaaccgagacgtgtacctgcagggtcccatctgggccaagattcctcacacggacggcaactttcacccgtctcctctgatgggcggctttggacttaaacacccgcctccacagatcctgatcaagaacacgccggtacctgcggatcctccaacaacgttcagccaggcgaaattggcttccttcatcacgcagtacagcaccggacaggtcagcgtggaaatcgagtgggagctgcagaaggagaacagcaaacgctggaacccagagattcagtacacttcaaactactacaaatctacaaatgtggactttgctgtcaatacagagggaacttattctgagcctcgccccattggtactcgttatctgacacgtaatctgtaa

SEQ ID NO: 105 - DNA, 2217 bp, Adeno-associated virus 10
atggctgctgacggttatcttccagattggctcgaggacaacctctctgagggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgttcccccgactcctccacgggcatcggcaagaaaggccagcagcccgctaaaaagagactgaactttgggcagactggcgagtcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatctccaacgggacatcgggaggaagcaccaacgacaacacctacttcggctacagcaccccctgggggtattttgacttcaacagattccactgccacttctcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggatgtcttcatgattccccagtacggctacctgacactgaacaatggaagtcaagccgtaggccgttcctccttctactgcctggaatattttccatctcaaatgctgcgaactggaaacaattttgaattcagctacaccttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggaagagactctctggtgaatcccggtgtcgccatggcaacccacaaggacgacgaggaacgcttcttcccgtcgagcggagtcctgatgtttggaaaacagggtgctggaagagacaatgtggactacagcagcgttatgctaacaagcgaagaagaaattaaaaccactaaccctgtagccacagaacaatacggcgtggtggctgacaacttgcagcaagccaatacagggcctattgtgggaaatgtcaacagccaaggagccttacctggcatggtctggcagaaccgagacgtgtacctgcagggtcccatctgggccaagattcctcacacggacggcaactttcacccgtctcctctgatgggcggctttggacttaaacacccgcctccacagatcctgatcaagaacacgccggtacctgcggatcctccaacaacgttcagccaggcgaaattggcttccttcatcacgcagtacagcaccggacaggtcagcgtggaaatcgagtgggagctgcagaaggagaacagcaaacgctggaacccagagattcagtacacttcaaactactacaaatctacaaatgtggactttgctgtcaatacagagggaacttattctgagcctcgccccattggtactcgttatctgacacgtaatctgtaa

SEQ ID NO: 106 - DNA, 4087 bp, Adeno-associated virus 11
atgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccactggcgccgcgtgagtaaggccccggaggccctcttctttgttcagttcgagaagggcgagtcctacttccacctccacgttctcgtcgagaccacgggggtcaagtccatggtcctgggccgcttcctgagtcagatcagagacaggctggtgcagaccatctaccgcggggtcgagcccacgctgcccaactggttcgcggtgaccaagacgcgaaatggcgccggcggggggaacaaggtggtggacgagtgctacatccccaactacctcctgcccaagacccagcccgagctgcagtgggcgtggactaacatggaggagtatataagcgcgtgtctaaacctcgcggagcgtaaacggctcgtggcgcagcacctgacccacgtcagccagacgcaggagcagaacaaggagaatctgaacccgaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggaaagatcatggcgctgaccaaatccgcgcccgactacctggtaggcccgtccttacccgcggacattaaggccaaccgcatctaccgcatcctggagctcaacggctacgaccccgcctacgccggctccgtcttcctgggctgggcgcagaaaaagttcggtaaacgcaacaccatctggctgtttgggcccgccaccaccggcaagaccaacatcgcggaagccatagcccacgccgtgcccttctacggctgcgtgaactggaccaatgagaactttcccttcaacgattgcgtcgacaagatggtgatctggtgggaggagggcaagatgaccgccaaggtcgtggagtccgccaaggccattctgggcggaagcaaggtgcgcgtggaccaaaagtgcaagtcctcggcccagatcgaccccacgcccgtgatcgtcacctccaacaccaacatgtgcgccgtgatcgacgggaacagcaccaccttcgagcaccagcagccgctgcaggaccgcatgttcaagttcgagctcacccgccgtctggagcacgactttggcaaggtgaccaagcaggaagtcaaagagttcttccgctgggctcaggatcacgtgactgaggtggcgcatgagttctacgtcagaaagggcggagccaccaaaagacccgcccccagtgacgcggatataagcgagcccaagcgggcctgcccctcagttccggagccatcgacgtcagacgcggaagcaccggtggactttgcggacaggtaccaaaacaaatgttctcgtcacgcgggcatgcttcagatgctgtttccctgcaagacatgcgagagaatgaatcagaatttcaacgtctgcttcacgcacggggtcagagactgctcagagtgcttccccggcgcgtcagaatctcaacccgtcgtcagaaaaaagacgtatcagaaactgtgcgcgattcatcatctgctggggcgggcacccgagattgcgtgttcggcctgcgatctcgtcaacgtggacttggatgactgtgtttctgagcaataaatgacttaaaccaggtatggctgctgacggttatcttccagattggctcgaggacaacctctctgagggcattcgcgagtggtgggacctgaaacctggagccccgaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggtactcgaacctctgggcctggttgaagaaggtgctaaaacggctcctggaaagaagagaccgttagagtcaccacaagagcccgactcctcctcgggcatcggcaaaaaaggcaaacaaccagccagaaagaggctcaactttgaagaggacactggagccggagacggaccccctgaaggatcagataccagcgccatgtcttcagacattgaaatgcgtgcagcaccgggcggaaatgctgtcgatgcgggacaaggttccgatggagtgggtaatgcctcgggtgattggcattgcgattccacctggtctgagggcaaggtcacaacaacctcgaccagaacctgggtcttgcccacctacaacaaccacttgtacctgcgtctcggaacaacatcaagcagcaacacctacaacggattctccaccccctggggatattttgacttcaacagattccactgtcacttctcaccacgtgactggcaaagactcatcaacaacaactggggactacgaccaaaagccatgcgcgttaaaatcttcaatatccaagttaaggaggtcacaacgtcgaacggcgagactacggtcgctaataaccttaccagcacggttcagatatttgcggactcgtcgtatgagctcccgtacgtgatggacgctggacaagaggggagcctgcctcctttccccaatgacgtgttcatggtgcctcaatatggctactgtggcatcgtgactggcgagaatcagaaccaaacggacagaaacgctttctactgcctggagtattttccttcgcaaatgttgagaactggcaacaactttgaaatggcttacaactttgagaaggtgccgttccactcaatgtatgctcacagccagagcctggacagactgatgaatcccctcctggaccagtacctgtggcacttacagtcgactacctctggagagactctgaatcaaggcaatgcagcaaccacatttggaaaaatcaggagtggagactttgccttttacagaaagaactggctgcctgggccttgtgttaaacagcagagattctcaaaaactgccagtcaaaattacaagattcctgccagcgggggcaacgctctgttaaagtatgacacccactataccttaaacaaccgctggagcaacatcgcgcccggacctccaatggccacagccggaccttcggatggggacttcagtaacgcccagcttatattccctggaccatctgttaccggaaatacaacaacttcagccaacaatctgttgtttacatcagaagaagaaattgctgccaccaacccaagagacacggacatgtttggccagattgctgacaataatcagaatgctacaactgctcccataaccggcaacgtgactgctatgggagtgctgcctggcatggtgtggcaaaacagagacatttactaccaagggccaatttgggccaagatcccacacgcggacggacattttcatccttcaccgctgattggtgggtttggactgaaacacccgcctccccagatattcatcaagaacactcccgtacctgccaatcctgcgacaaccttcactgcagccagagtggactctttcatcacacaatacagcaccggccaggtcgctgttcagattgaatgggaaattgaaaaggaacgctccaaacgctggaatcctgaagtgcagtttacttcaaactatgggaaccagtcttctatgttgtgggctcctgatacaactgggaagtatacagagccgcgggttattggctctcgttatttgactaatcatttgtaa

SEQ ID NO: 107 - DNA, 2202 bp, Adeno-associated virus 11
atggctgctgacggttatcttccagattggctcgaggacaacctctctgagggcattcgcgagtggtgggacctgaaacctggagccccgaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggtactcgaacctctgggcctggttgaagaaggtgctaaaacggctcctggaaagaagagaccgttagagtcaccacaagagcccgactcctcctcgggcatcggcaaaaaaggcaaacaaccagccagaaagaggctcaactttgaagaggacactggagccggagacggaccccctgaaggatcagataccagcgccatgtcttcagacattgaaatgcgtgcagcaccgggcggaaatgctgtcgatgcgggacaaggttccgatggagtgggtaatgcctcgggtgattggcattgcgattccacctggtctgagggcaaggtcacaacaacctcgaccagaacctgggtcttgcccacctacaacaaccacttgtacctgcgtctcggaacaacatcaagcagcaacacctacaacggattctccaccccctggggatattttgacttcaacagattccactgtcacttctcaccacgtgactggcaaagactcatcaacaacaactggggactacgaccaaaagccatgcgcgttaaaatcttcaatatccaagttaaggaggtcacaacgtcgaacggcgagactacggtcgctaataaccttaccagcacggttcagatatttgcggactcgtcgtatgagctcccgtacgtgatggacgctggacaagaggggagcctgcctcctttccccaatgacgtgttcatggtgcctcaatatggctactgtggcatcgtgactggcgagaatcagaaccaaacggacagaaacgctttctactgcctggagtattttccttcgcaaatgttgagaactggcaacaactttgaaatggcttacaactttgagaaggtgccgttccactcaatgtatgctcacagccagagcctggacagactgatgaatcccctcctggaccagtacctgtggcacttacagtcgactacctctggagagactctgaatcaaggcaatgcagcaaccacatttggaaaaatcaggagtggagactttgccttttacagaaagaactggctgcctgggccttgtgttaaacagcagagattctcaaaaactgccagtcaaaattacaagattcctgccagcgggggcaacgctctgttaaagtatgacacccactataccttaaacaaccgctggagcaacatcgcgcccggacctccaatggccacagccggaccttcggatggggacttcagtaacgcccagcttatattccctggaccatctgttaccggaaatacaacaacttcagccaacaatctgttgtttacatcagaagaagaaattgctgccaccaacccaagagacacggacatgtttggccagattgctgacaataatcagaatgctacaactgctcccataaccggcaacgtgactgctatgggagtgctgcctggcatggtgtggcaaaacagagacatttactaccaagggccaatttgggccaagatcccacacgcggacggacattttcatccttcaccgctgattggtgggtttggactgaaacacccgcctccccagatattcatcaagaacactcccgtacctgccaatcctgcgacaaccttcactgcagccagagtggactctttcatcacacaatacagcaccggccaggtcgctgttcagattgaatgggaaattgaaaaggaacgctccaaacgctggaatcctgaagtgcagtttacttcaaactatgggaaccagtcttctatgttgtgggctcctgatacaactgggaagtatacagagccgcgggttattggctctcgttatttgactaatcatttgtaa

SEQ ID NO: 108 - DNA, 4213 bp, Adeno-associated virus 12
ttgcgacagtttgcgacaccatgtggtcacaagaggtatataaccgcgagtgagccagcgaggagctccattttgcccgcgaagtttgaacgagcagcagccatgccggggttctacgaggtggtgatcaaggtgcccagcgacctggacgagcacctgcccggcatttctgactcctttgtgaactgggtggccgagaaggaatgggagttgcccccggattctgacatggatcagaatctgattgagcaggcacccctgaccgtggccgagaagctgcagcgcgagttcctggtggaatggcgccgagtgagtaaatttctggaggccaagttttttgtgcagtttgaaaagggggactcgtactttcatttgcatattctgattgaaattaccggcgtgaaatccatggtggtgggccgctacgtgagtcagattagggataaactgatccagcgcatctaccgcggggtcgagccccagctgcccaactggttcgcggtcacaaagacccgaaatggcgccggaggcgggaacaaggtggtggacgagtgctacatccccaactacctgctccccaaggtccagcccgagcttcagtgggcgtggactaacatggaggagtatataagcgcctgtttgaacctcgcggagcgtaaacggctcgtggcgcagcacctgacgcacgtctcccagacccaggagggcgacaaggagaatctgaacccgaattctgacgcgccggtgatccggtcaaaaacctccgccaggtacatggagctggtcgggtggctggtggacaagggcatcacgtccgagaagcagtggatccaggaggaccaggcctcgtacatctccttcaacgcggcctccaactcccggtcgcagatcaaggcggccctggacaatgcctccaaaatcatgagcctcaccaaaacggctccggactatctcatcgggcagcagcccgtgggggacattaccaccaaccggatctacaaaatcctggaactgaacgggtacgacccccagtacgccgcctccgtctttctcggctgggcccagaaaaagtttggaaagcgcaacaccatctggctgtttgggcccgccaccaccggcaagaccaacatcgcggaagccatcgcccacgcggtccccttctacggctgcgtcaactggaccaatgagaactttcccttcaacgactgcgtcgacaaaatggtgatttggtgggaggagggcaagatgaccgccaaggtcgtagagtccgccaaggccattctgggcggcagcaaggtgcgcgtggaccaaaaatgcaaggcctctgcgcagatcgaccccacccccgtgatcgtcacctccaacaccaacatgtgcgccgtgattgacgggaacagcaccaccttcgagcaccagcagcccctgcaggaccggatgttcaagtttgaactcacccgccgcctcgaccacgactttggcaaggtcaccaagcaggaagtcaaggactttttccggtgggcggctgatcacgtgactgacgtggctcatgagttttacgtcacaaagggtggagctaagaaaaggcccgccccctctgacgaggatataagcgagcccaagcggccgcgcgtgtcatttgcgcagccggagacgtcagacgcggaagctcccggagacttcgccgacaggtaccaaaacaaatgttctcgtcacgcgggtatgctgcagatgctctttccctgcaagacgtgcgagagaatgaatcagaattccaacgtctgcttcacgcacggtcagaaagattgcggggagtgctttcccgggtcagaatctcaaccggtttctgtcgtcagaaaaacgtatcagaaactgtgcatccttcatcagctccggggggcacccgagatcgcctgctctgcttgcgaccaactcaaccccgatttggacgattgccaatttgagcaataaatgactgaaatcaggtatggctgctgacggttatcttccagattggctcgaggacaacctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtatctcaagtacaaccacgccgacgccgagttccagcagcgcttggcgaccgacacctcttttgggggcaacctcgggcgagcagtcttccaggccaaaaagaggattctcgagcctctgggtctggttgaagagggcgttaaaacggctcctggaaagaaacgcccattagaaaagactccaaatcggccgaccaacccggactctgggaaggccccggccaagaaaaagcaaaaagacggcgaaccagccgactctgctagaaggacactcgactttgaagactctggagcaggagacggaccccctgagggatcatcttccggagaaatgtctcatgatgctgagatgcgtgcggcgccaggcggaaatgctgtcgaggcgggacaaggtgccgatggagtgggtaatgcctccggtgattggcattgcgattccacctggtcagagggccgagtcaccaccaccagcacccgaacctgggtcctacccacgtacaacaaccacctgtacctgcgaatcggaacaacggccaacagcaacacctacaacggattctccaccccctggggatactttgactttaaccgcttccactgccacttttccccacgcgactggcagcgactcatcaacaacaactggggactcaggccgaaatcgatgcgtgttaaaatcttcaacatacaggtcaaggaggtcacgacgtcaaacggcgagactacggtcgctaataaccttaccagcacggttcagatctttgcggattcgacgtatgaactcccatacgtgatggacgccggtcaggaggggagctttcctccgtttcccaacgacgtctttatggttccccaatacggatactgcggagttgtcactggaaaaaaccagaaccagacagacagaaatgccttttactgcctggaatactttccatcccaaatgctaagaactggcaacaattttgaagtcagttaccaatttgaaaaagttcctttccattcaatgtacgcgcacagccagagcctggacagaatgatgaatcctttactggatcagtacctgtggcatctgcaatcgaccactaccggaaattcccttaatcaaggaacagctaccaccacgtacgggaaaattaccactggagactttgcctactacaggaaaaactggttgcctggagcctgcattaaacaacaaaaattttcaaagaatgccaatcaaaactacaagattcccgccagcgggggagacgcccttttaaagtatgacacgcataccactctaaatgggcgatggagtaacatggctcctggacctccaatggcaaccgcaggtgccggggactcggattttagcaacagccagctgatctttgccggacccaatccgagcggtaacacgaccacatcttcaaacaatttgttgtttacctcagaagaggagattgccacaacaaacccacgagacacggacatgtttggacagattgcagataataatcaaaatgccaccaccgcccctcacatcgctaacctggacgctatgggaattgttcccggaatggtctggcaaaacagagacatctactaccagggccctatttgggccaaggtccctcacacggacggacactttcacccttcgccgctgatgggaggatttggactgaaacacccgcctccacagattttcatcaaaaacacccccgtacccgccaatcccaatactacctttagcgctgcaaggattaattcttttctgacgcagtacagcaccggacaagttgccgttcagatcgactgggaaattcagaaggagcattccaaacgctggaatcccgaagttcaatttacttcaaactacggcactcaaaattctatgctgtgggctcccgacaatgctggcaactaccacgaactccgggctattgggtcccgtttcctcacccaccacttgtaa

SEQ ID NO: 109 - DNA, 1866 bp, Adeno-associated virus 12
atgccggggttctacgaggtggtgatcaaggtgcccagcgacctggacgagcacctgcccggcatttctgactcctttgtgaactgggtggccgagaaggaatgggagttgcccccggattctgacatggatcagaatctgattgagcaggcacccctgaccgtggccgagaagctgcagcgcgagttcctggtggaatggcgccgagtgagtaaatttctggaggccaagttttttgtgcagtttgaaaagggggactcgtactttcatttgcatattctgattgaaattaccggcgtgaaatccatggtggtgggccgctacgtgagtcagattagggataaactgatccagcgcatctaccgcggggtcgagccccagctgcccaactggttcgcggtcacaaagacccgaaatggcgccggaggcgggaacaaggtggtggacgagtgctacatccccaactacctgctccccaaggtccagcccgagcttcagtgggcgtggactaacatggaggagtatataagcgcctgtttgaacctcgcggagcgtaaacggctcgtggcgcagcacctgacgcacgtctcccagacccaggagggcgacaaggagaatctgaacccgaattctgacgcgccggtgatccggtcaaaaacctccgccaggtacatggagctggtcgggtggctggtggacaagggcatcacgtccgagaagcagtggatccaggaggaccaggcctcgtacatctccttcaacgcggcctccaactcccggtcgcagatcaaggcggccctggacaatgcctccaaaatcatgagcctcaccaaaacggctccggactatctcatcgggcagcagcccgtgggggacattaccaccaaccggatctacaaaatcctggaactgaacgggtacgacccccagtacgccgcctccgtctttctcggctgggcccagaaaaagtttggaaagcgcaacaccatctggctgtttgggcccgccaccaccggcaagaccaacatcgcggaagccatcgcccacgcggtccccttctacggctgcgtcaactggaccaatgagaactttcccttcaacgactgcgtcgacaaaatggtgatttggtgggaggagggcaagatgaccgccaaggtcgtagagtccgccaaggccattctgggcggcagcaaggtgcgcgtggaccaaaaatgcaaggcctctgcgcagatcgaccccacccccgtgatcgtcacctccaacaccaacatgtgcgccgtgattgacgggaacagcaccaccttcgagcaccagcagcccctgcaggaccggatgttcaagtttgaactcacccgccgcctcgaccacgactttggcaaggtcaccaagcaggaagtcaaggactttttccggtgggcggctgatcacgtgactgacgtggctcatgagttttacgtcacaaagggtggagctaagaaaaggcccgccccctctgacgaggatataagcgagcccaagcggccgcgcgtgtcatttgcgcagccggagacgtcagacgcggaagctcccggagacttcgccgacaggtaccaaaacaaatgttctcgtcacgcgggtatgctgcagatgctctttccctgcaagacgtgcgagagaatgaatcagaattccaacgtctgcttcacgcacggtcagaaagattgcggggagtgctttcccgggtcagaatctcaaccggtttctgtcgtcagaaaaacgtatcagaaactgtgcatccttcatcagctccggggggcacccgagatcgcctgctctgcttgcgaccaactcaaccccgatttggacgattgccaatttgagcaataa

SEQ ID NO: 110 - DNA, 2229 bp, Adeno-associated virus 12
atggctgctgacggttatcttccagattggctcgaggacaacctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtatctcaagtacaaccacgccgacgccgagttccagcagcgcttggcgaccgacacctcttttgggggcaacctcgggcgagcagtcttccaggccaaaaagaggattctcgagcctctgggtctggttgaagagggcgttaaaacggctcctggaaagaaacgcccattagaaaagactccaaatcggccgaccaacccggactctgggaaggccccggccaagaaaaagcaaaaagacggcgaaccagccgactctgctagaaggacactcgactttgaagactctggagcaggagacggaccccctgagggatcatcttccggagaaatgtctcatgatgctgagatgcgtgcggcgccaggcggaaatgctgtcgaggcgggacaaggtgccgatggagtgggtaatgcctccggtgattggcattgcgattccacctggtcagagggccgagtcaccaccaccagcacccgaacctgggtcctacccacgtacaacaaccacctgtacctgcgaatcggaacaacggccaacagcaacacctacaacggattctccaccccctggggatactttgactttaaccgcttccactgccacttttccccacgcgactggcagcgactcatcaacaacaactggggactcaggccgaaatcgatgcgtgttaaaatcttcaacatacaggtcaaggaggtcacgacgtcaaacggcgagactacggtcgctaataaccttaccagcacggttcagatctttgcggattcgacgtatgaactcccatacgtgatggacgccggtcaggaggggagctttcctccgtttcccaacgacgtctttatggttccccaatacggatactgcggagttgtcactggaaaaaaccagaaccagacagacagaaatgccttttactgcctggaatactttccatcccaaatgctaagaactggcaacaattttgaagtcagttaccaatttgaaaaagttcctttccattcaatgtacgcgcacagccagagcctggacagaatgatgaatcctttactggatcagtacctgtggcatctgcaatcgaccactaccggaaattcccttaatcaaggaacagctaccaccacgtacgggaaaattaccactggagactttgcctactacaggaaaaactggttgcctggagcctgcattaaacaacaaaaattttcaaagaatgccaatcaaaactacaagattcccgccagcgggggagacgcccttttaaagtatgacacgcataccactctaaatgggcgatggagtaacatggctcctggacctccaatggcaaccgcaggtgccggggactcggattttagcaacagccagctgatctttgccggacccaatccgagcggtaacacgaccacatcttcaaacaatttgttgtttacctcagaagaggagattgccacaacaaacccacgagacacggacatgtttggacagattgcagataataatcaaaatgccaccaccgcccctcacatcgctaacctggacgctatgggaattgttcccggaatggtctggcaaaacagagacatctactaccagggccctatttgggccaaggtccctcacacggacggacactttcacccttcgccgctgatgggaggatttggactgaaacacccgcctccacagattttcatcaaaaacacccccgtacccgccaatcccaatactacctttagcgctgcaaggattaattcttttctgacgcagtacagcaccggacaagttgccgttcagatcgactgggaaattcagaaggagcattccaaacgctggaatcccgaagttcaatttacttcaaactacggcactcaaaattctatgctgtgggctcccgacaatgctggcaactaccacgaactccgggctattgggtcccgtttcctcacccaccacttgtaa

SEQ ID NO: 111 - DNA, 675 bp, Artificial Sequence
  Description of Artificial Sequence Synthetic polynucleotide
atgagtgtgattaaaccagacatgaagatcaagctgcgtatggaaggcgctgtaaatggacacccgttcgcgattgaaggagttggccttgggaagcctttcgagggaaaacagagtatggaccttaaagtcaaagaaggcggacctctgcctttcgcctatgacatcttgacaactgtgttctgttacggcaacagggtattcgccaaatacccagaaaatatagtagactatttcaagcagtcgtttcctgagggctactcttgggaacgaagcatgaattacgaagacgggggcatttgtaacgcgacaaacgacataaccctggatggtgactgttatatctatgaaattcgatttgatggtgtgaactttcctgccaatggtccagttatgcagaagaggactgtgaaatgggagccatccactgagaaattgtatgtgcgtgatggagtgctgaagggtgatgttaacatggctctgtcgcttgaaggaggtggccattaccgatgtgacttcaaaactacttataaagctaagaaggttgtccagttgccagactatcactttgtggaccaccacattgagattaaaagccacgacaaagattacagtaatgttaatctgcatgagcatgccgaagcgcattctgagctgccgaggcaggccaagtaa

SEQ ID NO: 112 - protein, 224 aa, Artificial Sequence
  Description of Artificial Sequence Synthetic polypeptide
MSVIKPDMKIKLRMEGAVNGHPFAIEGVGLGKPFEGKQSMDLKVKEGGPLPFAYDILTTVFCYGNRVFAKYPENIVDYFKQSFPEGYSWERSMNYEDGGICNATNDITLDGDCYIYEIRFDGVNFPANGPVMQKRTVKWEPSTEKLYVRDGVLKGDVNMALSLEGGGHYRCDFKTTYKAKKVVQLPDYHFVDHHIEIKSHDKDYSNVNLHEHAEAHSELPRQAK

SEQ ID NO: 113 - DNA, 1528 bp, Artificial Sequence
  Description of Artificial Sequence Synthetic polynucleotide
gtgggatctctgtgcaaagctacaatggagatctattgtatgaaccgtgggagatatactgcgaaaagggcaaaccttttacgagtttcaattcttactggaagaaatgcttagatatgtcgattgaatccgttatgcttcctcctccttggcggttgatgccaataactgcaggtaaaaccttaaagagtattacttaaaagctaaaacgtttttgatttcttcaggacataagcggtagtaaaagtttatggctttttctttgttagcggctgaagcgatttgggcgtgttcgattgaagaactagggctggagaatgaggccgagaaaccgagcaatgcgttgttaactagagcttggtctccaggatggagcaatgctgataagttactaaatgagttcatcgagaagcagttgatagattatgcaaagaacagcaagaaagttgttgggaattctacttcactactttctccgtatctccatttcggggaaataagcgtcagacacgttttccagtgtgcccggatgaaacaaattatatgggcaagagataagaacagtgaaggagaagaaagtgcagatctttttcttaggggaatcggtttaagagagtattctcggtatatatgtttcaacttcccgtttactcacgagcaatcgttgttgagtcatcttcggtttttcccttgggatgctgatgttgataagttcaaggcctggagacaaggcaggaccggttatccgttggtggatgccggaatgagagagctttgggctaccggatggatgcataacagaataagagtgattgtttcaagctttgctgtgaagtttcttctccttccatggaaatggggaatgaagtatttctgggatacacttttggatgctgatttggaatgtgacatccttggctggcagtatatctctgggagtatccccgatggccacgagcttgatcgcttggacaatcccgcggtaaactacaaaacttgtcttatagtttagaattcaaagcttaataccagtttttgctatgcattcgttttttattttatttttcagcttatttggttttggttgatttagttctgaagtctatgaaaactctgtttttatttcagttacaaggcgccaaatatgacccagaaggtgagtacataaggcaatggcttcccgagcttgcgagattgccaactgaatggatccatcatccatgggacgctcctttaaccgtactcaaagcttctggtgtggaactcggaacaaactatgcgaaacccattgtagacatcgacacagctcgtgagctactagctaaagctatttcaagaacccgtgaagcacagatcatgatcggagcagcacctgatgagattgtagcagatagcttcgaggccttaggggctaataccattaaagaacctggtctttgcccatctgtgtcttctaatgaccaacaagtaccttcggctgttcgttacaacgggtcaaagagagtgaaacctgaggaagaagaagaga

SEQ ID NO: 114 - DNA, 7582 bp, Artificial Sequence
  Description of Artificial Sequence Synthetic polynucleotide
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagatgcggccgatgatgttccttcctactgattattgttgcagactgagcgaccaggaatacatggaactcgtcttcgagaacggacagatactcgcaaaaggccagaggtcaaatgttagtctccataatcagcggacgaaaagcatcatggatctgtatgaggccgaatacaacgaagattttatgaaaagtattatccatggagggggtggcgctattaccaacctgggagatacccaagtggtcccacagtcccacgtagcagccgctcacgagaccaatatgctggagtccaacaaacacgtagacacgcgtgctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgctggctacaggcagtggcgcaccactggcagacaataacgagggcgccgacggagtgggtaattcctcgggaaattggcattgcgattccacatggctgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatgctctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 115 - DNA, 2793 bp, Artificial Sequence; identical to SEQ ID NO: 41
  Description of Artificial Sequence Synthetic polynucleotide

SEQ ID NO: 116 - protein, 930 aa, Artificial Sequence; identical to SEQ ID NO: 42
  Description of Artificial Sequence Synthetic polypeptide

SEQ ID NO: 117 - DNA, 6006 bp, Artificial Sequence
  Description of Artificial Sequence Synthetic polynucleotide
agctagcttctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctccctcgaggaactggaaaaccagaaagttaactggtaagtttagtctttttgtcttttatttcaggtcccggatcgaattgcggccgcccaccatggtttccggagtcgggggtagtggcggtggccgtggcggtggccgtggcggagaagaagaaccgtcgtcaagtcacactcctaataaccgaagaggaggagaacaagctcaatcgtcgggaacgaaatctctcagaccaagaagcaacactgaatcaatgagcaaagcaattcaacagtacaccgtcgacgcaagactccacgccgttttcgaacaatccggcgaatcagggaaatcattcgactactcacaatcactcaaaacgacgacgtacggttcctctgtacctgagcaacagatcacagcttatctctctcgaatccagcgaggtggttacattcagcctttcggatgtatgatcgccgtcgatgaatccagtttccggatcatcggttacagtgaaaacgccagagaaatgttagggattatgcctcaatctgttcctactcttgagaaacctgagattctagctatgggaactgatgtgagatctttgttcacttcttcgagctcgattctactcgagcgtgctttcgttgctcgagagattaccttgttaaatccggtttggatccattccaagaatactggtaaaccgttttacgccattcttcataggattgatgttggtgttgttattgatttagagccagctagaactgaagatcctgcgctttctattgctggtgctgttcaatcgcagaaactcgcggttcgtgcgatttctcagttacaggctcttcctggtggagatattaagcttttgtgtgacactgtcgtggaaagtgtgagggacttgactggttatgatcgtgttatggtttataagtttcatgaagatgagcatggagaagttgtagctgagagtaaacgagatgatttagagccttatattggactgcattatcctgctactgatattcctcaagcgtcaaggttcttgtttaagcagaaccgtgtccgaatgatagtagattgcaatgccacacctgttcttgtggtccaggacgataggctaactcagtctatgtgcttggttggttctactcttagggctcctcatggttgtcactctcagtatatggctaacatgggatctattgcgtctttagcaatggcggttataatcaatggaaatgaagatgatgggagcaatgtagctagtggaagaagctcgatgaggctttggggtttggttgtttgccatcacacttcttctcgctgcataccgtttccgctaaggtatgcttgtgagtttttgatgcaggctttcggtttacagttaaacatggaattgcagttagctttgcaaatgtcagagaaacgcgttttgagaacgcagacactgttatgtgatatgcttctgcgtgactcgcctgctggaattgttacacagagtcccagtatcatggacttagtgaaatgtgacggtgcagcatttctttaccacgggaagtattacccgttgggtgttgctcctagtgaagttcagataaaagatgttgtggagtggttgcttgcgaatcatgcggattcaaccggattaagcactgatagtttaggcgatgcggggtatcccggtgcagctgcgttaggggatgctgtgtgcggtatggcagttgcatatatcacaaaaagagactttcttttttggtttcgatctcacactgcgaaagaaatcaaatggggaggcgctaagcatcatccggaggataaagatgatgggcaacgaatgcatcctcgttcgtcctttcaggcttttcttgaagttgttaagagccggagtcagccatgggaaactgcggaaatggatgcgattcactcgctccagcttattctgagagactcttttaaagaatctgaggcggctatgaactctaaagttgtggatggtgtggttcagccatgtagggatatggcgggggaacaggggattgatgagttaggtgcagttgcaagagagatggttaggctcattgagactgcaactgttcctatattcgctgtggatgccggaggctgcatcaatggatggaacgctaagattgcagagttgacaggtctctcagttgaagaagctatggggaagtctctggtttctgatttaatatacaaagagaatgaagcaactgtcaataagcttctttctcgtgctttgagaggggacgaggaaaagaatgtggaggttaagctgaaaactttcagccccgaactacaagggaaagcagtttttgtggttgtgaatgcttgttccagcaaggactacttgaacaacattgtcggcgtttgttttgttggacaagacgttactagtcagaaaatcgtaatggataagttcatcaacatacaaggagattacaaggctattgtacatagcccaaaccctctaatcccgccaatttttgctgctgacgagaacacgtgctgcctggaatggaacatggcgatggaaaagcttacgggttggtctcgcagtgaagtgattgggaaaatgattgtcggggaagtgtttgggagctgttgcatgctaaagggtcctgatgctttaaccaagttcatgattgtattgcataatgcgattggtggccaagatacggataagttccctttcccattctttgaccgcaatgggaagtttgttcaggctctattgactgcaaacaagcgggttagcctcgagggaaaggttattggggctttctgtttcttgcaaatcccgagcgaattcgatagtgctggtagtgctggtagtgctggttccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagggggcgcgaggatcctctagagtcgacctgcagcccaagcttcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgatcgaccaattctcatgtttgacagcttatcatcgata

SEQ ID NO: 118 - DNA, 6012 bp, Artificial Sequence
  Description of Artificial Sequence Synthetic polynucleotide
agcttctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctccctcgaggaactggaaaaccagaaagttaactggtaagtttagtctttttgtcttttatttcaggtcccggatcgaattgcggccgcccaccatggtttccggagtcgggggtagtggcggtggccgtggcggtggccgtggcggagaagaagaaccgtcgtcaagtcacactcctaataaccgaagaggaggagaacaagctcaatcgtcgggaacgaaatctctcagaccaagaagcaacactgaatcaatgagcaaagcaattcaacagtacaccgtcgacgcaagactccacgccgttttcgaacaatccggcgaatcagggaaatcattcgactactcacaatcactcaaaacgacgacgtacggttcctctgtacctgagcaacagatcacagcttatctctctcgaatccagcgaggtggttacattcagcctttcggatgtatgatcgccgtcgatgaatccagtttccggatcatcggttacagtgaaaacgccagagaaatgttagggattatgcctcaatctgttcctactcttgagaaacctgagattctagctatgggaactgatgtgagatctttgttcacttcttcgagctcgattctactcgagcgtgctttcgttgctcgagagattaccttgttaaatccggtttggatccattccaagaatactggtaaaccgttttacgccattcttcataggattgatgttggtgttgttattgatttagagccagctagaactgaagatcctgcgctttctattgctggtgctgttcaatcgcagaaactcgcggttcgtgcgatttctcagttacaggctcttcctggtggagatattaagcttttgtgtgacactgtcgtggaaagtgtgagggacttgactggttatgatcgtgttatggtttataagtttcatgaagatgagcatggagaagttgtagctgagagtaaacgagatgatttagagccttatattggactgcattatcctgctactgatattcctcaagcgtcaaggttcttgtttaagcagaaccgtgtccgaatgatagtagattgcaatgccacacctgttcttgtggtccaggacgataggctaactcagtctatgtgcttggttggttctactcttagggctcctcatggttgtcactctcagtatatggctaacatgggatctattgcgtctttagcaatggcggttataatcaatggaaatgaagatgatgggagcaatgtagctagtggaagaagctcgatgaggctttggggtttggttgtttgccatcacacttcttctcgctgcataccgtttccgctaaggtatgcttgtgagtttttgatgcaggctttcggtttacagttaaacatggaattgcagttagctttgcaaatgtcagagaaacgcgttttgagaacgcagacactgttatgtgatatgcttctgcgtgactcgcctgctggaattgttacacagagtcccagtatcatggacttagtgaaatgtgacggtgcagcatttctttaccacgggaagtattacccgttgggtgttgctcctagtgaagttcagataaaagatgttgtggagtggttgcttgcgaatcatgcggattcaaccggattaagcactgatagtttaggcgatgcggggtatcccggtgcagctgcgttaggggatgctgtgtgcggtatggcagttgcatatatcacaaaaagagactttcttttttggtttcgatctcacactgcgaaagaaatcaaatggggaggcgctaagcatcatccggaggataaagatgatgggcaacgaatgcatcctcgttcgtcctttcaggcttttcttgaagttgttaagagccggagtcagccatgggaaactgcggaaatggatgcgattcactcgctccagcttattctgagagactcttttaaagaatctgaggcggctatgaactctaaagttgtggatggtgtggttcagccatgtagggatatggcgggggaacaggggattgatgagttaggtgcagttgcaagagagatggttaggctcattgagactgcaactgttcctatattcgctgtggatgccggaggctgcatcaatggatggaacgctaagattgcagagttgacaggtctctcagttgaagaagctatggggaagtctctggtttctgatttaatatacaaagagaatgaagcaactgtcaataagcttctttctcgtgctttgagaggggacgaggaaaagaatgtggaggttaagctgaaaactttcagccccgaactacaagggaaagcagtttttgtggttgtgaatgcttgttccagcaaggactacttgaacaacattgtcggcgtttgttttgttggacaagacgttactagtcagaaaatcgtaatggataagttcatcaacatacaaggagattacaaggctattgtacatagcccaaaccctctaatcccgccaatttttgctgctgacgagaacacgtgctgcctggaatggaacatggcgatggaaaagcttacgggttggtctcgcagtgaagtgattgggaaaatgattgtcggggaagtgtttgggagctgttgcatgctaaagggtcctgatgctttaaccaagttcatgattgtattgcataatgcgattggtggccaagatacggataagttccctttcccattctttgaccgcaatgggaagtttgttcaggctctattgactgcaaacaagcgggttagcctcgagggaaaggttattggggctttctgtttcttgcaaatcccgagcgaattcgatagtgctggtagtgctggtagtgctggttccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggcccaagaaaaagcggaaggtgtgatctagagtcgacctgcagcccaagcttcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgatcgaccaattctcatgtttgacagcttatcatcgataagct

SEQ ID NO: 119 - DNA, 5238 bp, Artificial Sequence
  Description of Artificial Sequence Synthetic polynucleotide
agcttctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctccctcgaggaactggaaaaccagaaagttaactggtaagtttagtctttttgtcttttatttcaggtcccggatcgaattgcggccgcccaccatggtttccggagtcgggggtagtggcggtggccgtggcggtggccgtggcggagaagaagaaccgtcgtcaagtcacactcctaataaccgaagaggaggagaacaagctcaatcgtcgggaacgaaatctctcagaccaagaagcaacactgaatcaatgagcaaagcaattcaacagtacaccgtcgacgcaagactccacgccgttttcgaacaatccggcgaatcagggaaatcattcgactactcacaatcactcaaaacgacgacgtacggttcctctgtacctgagcaacagatcacagcttatctctctcgaatccagcgaggtggttacattcagcctttcggatgtatgatcgccgtcgatgaatccagtttccggatcatcggttacagtgaaaacgccagagaaatgttagggattatgcctcaatctgttcctactcttgagaaacctgagattctagctatgggaactgatgtgagatctttgttcacttcttcgagctcgattctactcgagcgtgctttcgttgctcgagagattaccttgttaaatccggtttggatccattccaagaatactggtaaaccgttttacgccattcttcataggattgatgttggtgttgttattgatttagagccagctagaactgaagatcctgcgctttctattgctggtgctgttcaatcgcagaaactcgcggttcgtgcgatttctcagttacaggctcttcctggtggagatattaagcttttgtgtgacactgtcgtggaaagtgtgagggacttgactggttatgatcgtgttatggtttataagtttcatgaagatgagcatggagaagttgtagctgagagtaaacgagatgatttagagccttatattggactgcattatcctgctactgatattcctcaagcgtcaaggttcttgtttaagcagaaccgtgtccgaatgatagtagattgcaatgccacacctgttcttgtggtccaggacgataggctaactcagtctatgtgcttggttggttctactcttagggctcctcatggttgtcactctcagtatatggctaacatgggatctattgcgtctttagcaatggcggttataatcaatggaaatgaagatgatgggagcaatgtagctagtggaagaagctcgatgaggctttggggtttggttgtttgccatcacacttcttctcgctgcataccgtttccgctaaggtatgcttgtgagtttttgatgcaggctttcggtttacagttaaacatggaattgcagttagctttgcaaatgtcagagaaacgcgttttgagaacgcagacactgttatgtgatatgcttctgcgtgactcgcctgctggaattgttacacagagtcccagtatcatggacttagtgaaatgtgacggtgcagcatttctttaccacgggaagtattacccgttgggtgttgctcctagtgaagttcagataaaagatgttgtggagtggttgcttgcgaatcatgcggattcaaccggattaagcactgatagtttaggcgatgcggggtatcccggtgcagctgcgttaggggatgctgtgtgcggtatggcagttgcatatatcacaaaaagagactttcttttttggtttcgatctcacactgcgaaagaaatcaaatggggaggcgctaagcatcatccggaggataaagatgatgggcaacgaatgcatcctcgttcgtcctttcaggcttttcttgaagttgttaagagccggagtcagccatgggaaactgcggaaatggatgcgattcactcgctccagcttattctgagagactcttttaaagaatctgaggcggctatgaactctaaagttgtggatggtgtggttcagccatgtagggatatggcgggggaacaggggattgatgagttaggtgaattcgatagtgctggtagtgctggtagtgctggttccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggcccaagaaaaagcggaaggtgtgatctagagtcgacctgcagcccaagcttcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgatcgaccaattctcatgtttgacagcttatcatcgataagct

SEQ ID NO: 120 - DNA, 304 bp, Arabidopsis thaliana
gccgatgatgttccttcctactgattattgttgcagactgagcgaccaggaatacatggaactcgtcttcgagaacggacagatactcgcaaaaggccagaggtcaaatgttagtctccataatcagcggacgaaaagcatcatggatctgtatgaggccgaatacaacgaagattttatgaaaagtattatccatggagggggtggcgctattaccaacctgggagatacccaagtggtcccacagtcccacgtagcagccgctcacgagaccaatatgctggagtccaacaaacacgtagac

SEQ ID NO: 121 - protein, 100 aa, Arabidopsis thaliana
MMFLPTDYCCRLSDQEYMELVFENGQILAKGQRSNVSLHNQRTKSIMDLYEAEYNEDFMKSIIHGGGGAITNLGDTQVVPQSHVAAAHETNMLESNKHVD

SEQ ID NO: 122 - DNA, 2793 bp, Artificial Sequence
  Description of Artificial Sequence Synthetic polynucleotide
atgggtgcttcaggtgtatctggtgttggtggttctggtggtggaagaggtggaggtagaggaggtgaagaagaaccatcaagtagtcatacacctaacaatcgtagaggtggtgagcaagctcaatcatcaggtacaaaatcattacgtccaagaagtaatactgaatcaatgtcaaaagcaattcaacaatacacagtagatgctagattacacgccgtattcgaacaatctggagaaagtggtaagagttttgattactcacaatcattgaaaacaaccacttatggtagttcagttccagaacaacaaatcactgcatatcttagtagaatacaacgtggtggttacattcaaccatttggttgtatgattgcagttgatgaatcttcttttagaatcattggttattcagaaaatgcaagagaaatgttgggtatcatgccacaatcagtaccaaccttagaaaaaccagaaattcttgcaatgggtacagatgttagaagtttgtttacatcatcatcatcaattcttttggagagagcttttgttgcacgtgaaatcactttacttaatccagtatggattcatagtaagaatactggaaagccattctatgcaattcttcatagaatagatgtaggagttgttattgatcttgagccagcaagaacagaagatccagcattatctattgctggtgcagtacaatcacaaaaacttgctgttagagcaattagtcaattacaagccttgccaggtggtgatataaaacttctttgtgatacagttgttgaatcagttcgtgatcttaccggttatgatagagttatggtacacaaattccatgaggatgaacatggtgaagttgttgcagaaagtaaaagagatgatcttgaaccatacattggtttgcattatccagctactgatattccacaagcatcaagatttcttttcaaacaaaatcgtgttagaatgattgtagattgtaatgccaccccagtattagttgttcaagatgatagattgacacaaagtatgtgtttagtaggttcaacattaagagcacctcatggatgtcattcacaatatatggccaatatgggttcaatagcatcattagctatggcagtaatcatcaatggaaatgaagatgatggttcaaatgttgcatcaggtagaagttcaatgcgtttatggggtttagtagtttgtcatcatacaagttctcgttgtatcccatttcctttacgttatgcatgtgaatttcttatgcaagcatttggtttacaattgaatatggaacttcaattagcattacaaatgagtgaaaagagagttttacgtacacaaacattgttatgcgatatgttattgagagattctccagctggtattgttactcaatcaccatctatcatggatcttgtaaagtgtgatggtgcagcattcttataccacggaaagtactatccattaggtgttgcaccatctgaagttcaaatcaaagatgttgtagaatggttattggctaatcacgcagattctactggtttatcaactgattctcttggtgatgctggttatcctggtgccgcagccttaggagatgctgtatgtggtatggccgttgcttacattacaaaaagagatttcttgttttggtttcgttctcatacagctaaagagatcaaatggggtggtgcaaaacatcatccagaagataaggatgatggtcaaagaatgcatccaagatcatcatttcaagcattcttagaagtagttaagtcaagaagtcaaccttgggaaacagcagaaatggatgcaatacattcattacaattgatacttcgtgattcattcaaagaatcagaagcagcaatgaatagtaaagttgttgatggtgttgttcaaccatgtagagatatggccggtgaacaaggtattgatgaattaggtgctgtagctagagaaatggttagattgatagaaactgccactgttccaatcttcgctgttgatgctggtggatgcataaacggttggaatgctaagatcgcagaattgaccggtttgtcagttgaagaagctatgggtaaaagtttagtttcagatttgatctataaggaaaatgaagcaaccgttaacaaattgttatcaagagcattgagaggagatgaggaaaagaatgtagaagttaagttaaagacattttcaccagagttacaaggtaaagcagtttttgttgtagttaatgcttgttcatcaaaagattacttgaataacattgtaggtgtttgttttgttggtcaagatgtaacttcacaaaagattgttatggataagtttatcaatatccaaggtgattacaaagctattgttcattctccaaatccattgattccaccaatctttgcagctgatgagaatacatgttgtttagaatggaatatggcaatggaaaagttaactggttggtcacgttcagaagtaattggtaagatgattgttggagaggtttttggtagttgttgtatgcttaaaggtccagatgctttaactaagtttatgattgttttgcataatgcaattggtggtcaagatacagataagttcccattccctttcttcgatagaaatggaaagtttgttcaagcattacttactgctaacaaaagagtatcattagaaggtaaagtaataggagctttttgtttcttacaaattccttcaccagaattacaacaagctcttgcagtaggtggtagtcatcatcatcatcatcattaa

SEQ ID NO: 123 - protein, 930 aa, Artificial Sequence; identical to SEQ ID NO: 42
  Description of Artificial Sequence Synthetic polypeptide

SEQ ID NO: 124 - DNA, 9597 bp, Artificial Sequence
  Description of Artificial Sequence Synthetic polynucleotide
actcgagactagagctagataaaaaaaatttttatttatttttatttattttgaattaaatagattacaaattaattaatcccatcaaatctttaaaaaaaaatggtttaaaaaaacttgggttggttaattattatttgaaaattttaaaacccaaattaaaaaaaaaaaatgggattcaaaaatttttttttttttttttttttttttttttttttttttttttttttcagattgcataaaaagattttttttttttttttttcttatttcttaaaacaaataaattaaattaaaaaataaaaaatggtatctggtgttggtggttctggtggtggaagaggtggaggtagaggaggtgaagaagaaccatcaagtagtcatacacctaacaatcgtagaggtggtgagcaagctcaatcatcaggtacaaaatcattacgtccaagaagtaatactgaatcaatgtcaaaagcaattcaacaatacacagtagatgctagattacacgccgtattcgaacaatctggagaaagtggtaagagttttgattactcacaatcattgaaaacaaccacttatggtagttcagttccagaacaacaaatcactgcatatcttagtagaatacaacgtggtggttacattcaaccatttggttgtatgattgcagttgatgaatcttcttttagaatcattggttattcagaaaatgcaagagaaatgttgggtatcatgccacaatcagtaccaaccttagaaaaaccagaaattcttgcaatgggtacagatgttagaagtttgtttacatcatcatcatcaattcttttggagagagcttttgttgcacgtgaaatcactttacttaatccagtatggattcatagtaagaatactggaaagccattctatgcaattcttcatagaatagatgtaggagttgttattgatcttgagccagcaagaacagaagatccagcattatctattgctggtgcagtacaatcacaaaaacttgctgttagagcaattagtcaattacaagccttgccaggtggtgatataaaacttctttgtgatacagttgttgaatcagttcgtgatcttaccggttatgatagagttatggtatacaaattccatgaggatgaacatggtgaagttgttgcagaaagtaaaagagatgatcttgaaccatacattggtttgcattatccagctactgatattccacaagcatcaagatttcttttcaaacaaaatcgtgttagaatgattgtagattgtaatgccaccccagtattagttgttcaagatgatagattgacacaaagtatgtgtttagtaggttcaacattaagagcacctcatggatgtcattcacaatatatggccaatatgggttcaatagcatcattagctatggcagtaatcatcaatggaaatgaagatgatggttcaaatgttgcatcaggtagaagttcaatgcgtttatggggtttagtagtttgtcatcatacaagttctcgttgtatcccatttcctttacgttatgcatgtgaatttcttatgcaagcatttggtttacaattgaatatggaacttcaattagcattacaaatgagtgaaaagagagttttacgtacacaaacattgttatgcgatatgttattgagagattctccagctggtattgttactcaatcaccatctatcatggatcttgtaaagtgtgatggtgcagcattcttataccacggaaagtactatccattaggtgttgcaccatctgaagttcaaatcaaagatgttgtagaatggttattggctaatcacgcagattctactggtttatcaactgattctcttggtgatgctggttatcctggtgccgcagccttaggagatgctgtatgtggtatggccgttgcttacattacaaaaagagatttcttgttttggtttcgttctcatacagctaaagagatcaaatggggtggtgcaaaacatcatccagaagataaggatgatggtcaaagaatgcatccaagatcatcatttcaagcattcttagaagtagttaagtcaagaagtcaaccttgggaaacagcagaaatggatgcaatacattcattacaattgatacttcgtgattcattcaaagaatcagaagcagcaatgaatagtaaagttgttgatggtgttgttcaaccatgtagagatatggccggtgaacaaggtattgatgaattaggtgctgtagctagagaaatggttagattgatagaaactgccactgttccaatcttcgctgttgatgctggtggatgcataaacggttggaatgctaagatcgcagaattgaccggtttgtcagttgaagaagctatgggtaaaagtttagtttcagatttgatctataaggaaaatgaagcaaccgttaacaaattgttatcaagagcattgagaggagatgaggaaaagaatgtagaagttaagttaaagacattttcaccagagttacaaggtaaagcagtttttgttgtagttaatgcttgttcatcaaaagattacttgaataacattgtaggtgtttgttttgttggtcaagatgtaacttcacaaaagattgttatggataagtttatcaatatccaaggtgattacaaagctattgttcattctccaaatccattgattccaccaatctttgcagctgatgagaatacatgttgtttagaatggaatatggcaatggaaaagttaactggttggtcacgttcagaagtaattggtaagatgattgttggagaggtttttggtagttgttgtatgcttaaaggtccagatgctttaactaagtttatgattgttttgcataatgcaattggtggtcaagatacagataagttcccattccctttcttcgatagaaatggaaagtttgttcaagcattacttactgctaacaaaagagtatcattagaaggtaaagtaataggagctttttgtttcttacaaattccttcaccagaattacaacaagctcttgcagtaggtgcttcaggtcatcatcatcatcatcattaaattatttaataaataataaaaaaacaaattgttgtaataatctaatattttcttttttttttaattttttttttttaaatcttaataattattaagttattttaattttttttttttttttttttttttttttttttttttttctatcaaaaaaatcaaatatatttaaaaaatttattatttacagatacattttgaatggtgaagataaatatatgcattagatgtaaaacagccaaagagtatgaaaatcaaaaagataaagcttatcgatttcgaaaaagtaaatagcaattattacaaaattcaatccgaatctacccaaataaattccaatgaaattgccgatttaaaaaagtttattaaagaagaagtcaataaaacttcttccaaaattgatttctttttagtttcttcaacagatgccctttcaaatccagaaaattattctctcttagaagtaaagtgtattaattgtcattctttgtgtcaaggaaaaaatttatatatttcatgtacaagagatggatgtcaaaacaatatttgctataattgtttaggaataaacataaacatatataatgttgttattaattctaaactttgccctccatgtttcaatgattcggtaatcaacaagaagtgtgccatgtgtagtaagaacggaactaaatgtaatttgaaccaagaatgtaaacttcatctttgtgcacagtgttctaaaaagtgtctatacattctgagagtcaaaactaattaaataaaatataaacttaatttctaaataaactcatttaaaaatatttaaataatatgaatttataactgtaattattgtattaaaaaattatataattatttaatgttaaaaatgtattaaaataattataaaaaaatataacaaaaattttcgtaaaaataatttgtaaaaaagctattaaaaatattatgaaaaaaaaattaaaaaaattattaaattgtttttgtaattaagctattaaaataattataaaaaaaaaatttttaaaattttaaaaatattttttgtaaaaaagtattaaaataattatgaaaaaaaaattttctaaaaaattaaaaaaaaaattaaaatatattttatgttaaaaacgtattaaaataactattaaaaaaattatatttaaaaaagtattaacttttttttaggtgtggttgtggggtggggtttaatatattataataaaaaattattttttgttcatttattattttcattgtatataatgtactcaacaacgttattattttttctttttttttttattgtatcaaaatcttctgttcttcaaaatgatcagattgaagtaaaatattttcaacttcttattgttatgtatcaaaaagaaaactgtgttgaaaagtcaatgacaggcgccgtaatttatgatgaatgtaatattcatggaagagttgaaacaaatagtactcatgcgcttttttatgatgacattgaaacaaataattcaagatgtaacaattttcgtaatttaacaaacttaattaaacttaatgaatgtattaatgacgagtttggagagtctattctttataaagaatataatgaaactgatgatggttatttgtttagagtggaagacagctttgttgaaattacttctctttcaatggattgtacaaaaaatagtaaaacaattattgaaaaattcaacatttgttcaaaatttgaaaatgtatatcatattacaaacattacacaagagaaatccaatagatttacatgtacagatccattgtgccactattgtaagaatgaaaacattcaaaacaatcttgattttaaaacaacaaagtgtactccaaagtatggtgcatctgattctgaatttttatcaacaatttacaatccaaagctcgatggctcaaataacggtatggaaaagtcagtaactcaagaaaaaaacatttcaaataatttaaaaattaatatatatttaattttctttttaattatttttttaattaaataaagttttattattttttaagagtaattattgctcttttttcatttgaaacaccagaagctaaacgtaattgttgttgactgaaattttttattttttttggggtaataggatttccttttttatgaagattaatatctttgactcgtgaaacattctttttaacttttgttttttctgttggtttatcatttgttttttcactaatttcaataccatcttgacgttcattcataacttcatcttttttttttcctgtttctgtatcttcttctatttttttttctttatctttttctttatcttcttcttgttcttcctcttctttttcttcttctgatactgcaggtgtttcttcttcttcttcttccgatattgtcggtttttctacttcttcttcttgttcttcttcttcttcttcttcttcttcttcttcttcttcttcttcttcttcttccggtaatttattaattatatttctttttttatatgaattacgtttggtttgtgcagtaatttccttacatagagtgcagctttcaagaaaaatttcaatttcttcgtttgttgcataataaccactgtctttgatatgattaaacatttttgattttcttaaatgctttccttctttaatatgaaaattatcgaattctaattcattaagaacaataagctcccctaatttaaaaaattagttaaaataaattaaaatgaacatgtataaagatggattttaccattttttgaaattctaaataacttttcttcatctccaatctttttgactgaaaaacgatttttaattgaagttattgttctgtgagtgttttgaatcgcccatttctctaaatcagtttgagatagtgttttataatctgaattgttatacacaacttttgctctattaaccaaatatttaaagatttcatcatcaactgaatattttgactttacgattcttgtccaaaaaacaatttctactactatcattttttatttataaaataatttaaatacaaaaatgaatttttttttttttaaaaaaaaaaaaatttgaaaaaaaaaaaaaaaaaattttaaaaaaaaaaaaaaaaaaaaaaaaaaaaaaaaaatcaaataaaaagtaaaaaataaaaaccgaaaaacattcattgtaatttcaaatgtcgaggccggcagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcgggatccattttatttaatatactaaataataaaaaagttaaaaaatgatcattggataaattttttataattataaataaagataataattttttttttaacaaaactaaaaataaaaataataaaataattgttaaaataggttttttttttttttttttttttttaataaatggtatttattaatttatttgttgtgtgtgtttttttttttataatatttttttttttagcattgaattaagaagaaatcaaattgattctagttcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccgtccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagcttgaacatcttcaccatccattttggatcttttatattatatttatttattgattatttttttgaattaattaaaaaaaaaaaaaatttcattttataatctcagaaacctcaaaaaaaaaaaaataaaaaataaaaaatataaaaaaataaaaataaaatcccaattttaaagcgaaaaaccacccatggtttgaaaatttcaatcaatttcaaataactttacttaaaaaaaacccattttttatttaaaaa

SEQ ID NO: 125 - DNA, 9597 bp, Artificial Sequence
  Description of Artificial Sequence Synthetic polynucleotide
actcgagactagagctagataaaaaaaatttttatttatttttatttattttgaattaaatagattacaaattaattaatcccatcaaatctttaaaaaaaaatggtttaaaaaaacttgggttggttaattattatttgaaaattttaaaacccaaattaaaaaaaaaaaatgggattcaaaaatttttttttttttttttttttttttttttttttttttttttttttcagattgcataaaaagattttttttttttttttttcttatttcttaaaacaaataaattaaattaaaaaataaaaaatggtatctggtgttggtggttctggtggtggaagaggtggaggtagaggaggtgaagaagaaccatcaagtagtcatacacctaacaatcgtagaggtggtgagcaagctcaatcatcaggtacaaaatcattacgtccaagaagtaatactgaatcaatgtcaaaagcaattcaacaatacacagtagatgctagattacacgccgtattcgaacaatctggagaaagtggtaagagttttgattactcacaatcattgaaaacaaccacttatggtagttcagttccagaacaacaaatcactgcatatcttagtagaatacaacgtggtggttacattcaaccatttggttgtatgattgcagttgatgaatcttcttttagaatcattggttattcagaaaatgcaagagaaatgttgggtatcatgccacaatcagtaccaaccttagaaaaaccagaaattcttgcaatgggtacagatgttagaagtttgtttacatcatcatcatcaattcttttggagagagcttttgttgcacgtgaaatcactttacttaatccagtatggattcatagtaagaatactggaaagccattctatgcaattcttcatagaatagatgtaggagttgttattgatcttgagccagcaagaacagaagatccagcattatctattgctggtgcagtacaatcacaaaaacttgctgttagagcaattagtcaattacaagccttgccaggtggtgatataaaacttctttgtgatacagttgttgaatcagttcgtgatcttaccggttatgatagagttatggtacacaaattccatgaggatgaacatggtgaagttgttgcagaaagtaaaagagatgatcttgaaccatacattggtttgcattatccagctactgatattccacaagcatcaagatttcttttcaaacaaaatcgtgttagaatgattgtagattgtaatgccaccccagtattagttgttcaagatgatagattgacacaaagtatgtgtttagtaggttcaacattaagagcacctcatggatgtcattcacaatatatggccaatatgggttcaatagcatcattagctatggcagtaatcatcaatggaaatgaagatgatggttcaaatgttgcatcaggtagaagttcaatgcgtttatggggtttagtagtttgtcatcatacaagttctcgttgtatcccatttcctttacgttatgcatgtgaatttcttatgcaagcatttggtttacaattgaatatggaacttcaattagcattacaaatgagtgaaaagagagttttacgtacacaaacattgttatgcgatatgttattgagagattctccagctggtattgttactcaatcaccatctatcatggatcttgtaaagtgtgatggtgcagcattcttataccacggaaagtactatccattaggtgttgcaccatctgaagttcaaatcaaagatgttgtagaatggttattggctaatcacgcagattctactggtttatcaactgattctcttggtgatgctggttatcctggtgccgcagccttaggagatgctgtatgtggtatggccgttgcttacattacaaaaagagatttcttgttttggtttcgttctcatacagctaaagagatcaaatggggtggtgcaaaacatcatccagaagataaggatgatggtcaaagaatgcatccaagatcatcatttcaagcattcttagaagtagttaagtcaagaagtcaaccttgggaaacagcagaaatggatgcaatacattcattacaattgatacttcgtgattcattcaaagaatcagaagcagcaatgaatagtaaagttgttgatggtgttgttcaaccatgtagagatatggccggtgaacaaggtattgatgaattaggtgctgtagctagagaaatggttagattgatagaaactgccactgttccaatcttcgctgttgatgctggtggatgcataaacggttggaatgctaagatcgcagaattgaccggtttgtcagttgaagaagctatgggtaaaagtttagtttcagatttgatctataaggaaaatgaagcaaccgttaacaaattgttatcaagagcattgagaggagatgaggaaaagaatgtagaagttaagttaaagacattttcaccagagttacaaggtaaagcagtttttgttgtagttaatgcttgttcatcaaaagattacttgaataacattgtaggtgtttgttttgttggtcaagatgtaacttcacaaaagattgttatggataagtttatcaatatccaaggtgattacaaagctattgttcattctccaaatccattgattccaccaatctttgcagctgatgagaatacatgttgtttagaatggaatatggcaatggaaaagttaactggttggtcacgttcagaagtaattggtaagatgattgttggagaggtttttggtagttgttgtatgcttaaaggtccagatgctttaactaagtttatgattgttttgcataatgcaattggtggtcaagatacagataagttcccattccctttcttcgatagaaatggaaagtttgttcaagcattacttactgctaacaaaagagtatcattagaaggtaaagtaataggagctttttgtttcttacaaattccttcaccagaattacaacaagctcttgcagtaggtgcttcaggtcatcatcatcatcatcattaaattatttaataaataataaaaaaacaaattgttgtaataatctaatattttcttttttttttaattttttttttttaaatcttaataattattaagttattttaattttttttttttttttttttttttttttttttttttttctatcaaaaaaatcaaatatatttaaaaaatttattatttacagatacattttgaatggtgaagataaatatatgcattagatgtaaaacagccaaagagtatgaaaatcaaaaagataaagcttatcgatttcgaaaaagtaaatagcaattattacaaaattcaatccgaatctacccaaataaattccaatgaaattgccgatttaaaaaagtttattaaagaagaagtcaataaaacttcttccaaaattgatttctttttagtttcttcaacagatgccctttcaaatccagaaaattattctctcttagaagtaaagtgtattaattgtcattctttgtgtcaaggaaaaaatttatatatttcatgtacaagagatggatgtcaaaacaatatttgctataattgtttaggaataaacataaacatatataatgttgttattaattctaaactttgccctccatgtttcaatgattcggtaatcaacaagaagtgtgccatgtgtagtaagaacggaactaaatgtaatttgaaccaagaatgtaaacttcatctttgtgcacagtgttctaaaaagtgtctatacattctgagagtcaaaactaattaaataaaatataaacttaatttctaaataaactcatttaaaaatatttaaataatatgaatttataactgtaattattgtattaaaaaattatataattatttaatgttaaaaatgtattaaaataattataaaaaaatataacaaaaattttcgtaaaaataatttgtaaaaaagctattaaaaatattatgaaaaaaaaattaaaaaaattattaaattgtttttgtaattaagctattaaaataattataaaaaaaaaatttttaaaattttaaaaatattttttgtaaaaaagtattaaaataattatgaaaaaaaaattttctaaaaaattaaaaaaaaaattaaaatatattttatgttaaaaacgtattaaaataactattaaaaaaattatatttaaaaaagtattaacttttttttaggtgtggttgtggggtggggtttaatatattataataaaaaattattttttgttcatttattattttcattgtatataatgtactcaacaacgttattattttttctttttttttttattgtatcaaaatcttctgttcttcaaaatgatcagattgaagtaaaatattttcaacttcttattgttatgtatcaaaaagaaaactgtgttgaaaagtcaatgacaggcgccgtaatttatgatgaatgtaatattcatggaagagttgaaacaaatagtactcatgcgcttttttatgatgacattgaaacaaataattcaagatgtaacaattttcgtaatttaacaaacttaattaaacttaatgaatgtattaatgacgagtttggagagtctattctttataaagaatataatgaaactgatgatggttatttgtttagagtggaagacagctttgttgaaattacttctctttcaatggattgtacaaaaaatagtaaaacaattattgaaaaattcaacatttgttcaaaatttgaaaatgtatatcatattacaaacattacacaagagaaatccaatagatttacatgtacagatccattgtgccactattgtaagaatgaaaacattcaaaacaatcttgattttaaaacaacaaagtgtactccaaagtatggtgcatctgattctgaatttttatcaacaatttacaatccaaagctcgatggctcaaataacggtatggaaaagtcagtaactcaagaaaaaaacatttcaaataatttaaaaattaatatatatttaattttctttttaattatttttttaattaaataaagttttattattttttaagagtaattattgctcttttttcatttgaaacaccagaagctaaacgtaattgttgttgactgaaattttttattttttttggggtaataggatttccttttttatgaagattaatatctttgactcgtgaaacattctttttaacttttgttttttctgttggtttatcatttgttttttcactaatttcaataccatcttgacgttcattcataacttcatcttttttttttcctgtttctgtatcttcttctatttttttttctttatctttttctttatcttcttcttgttcttcctcttctttttcttcttctgatactgcaggtgtttcttcttcttcttcttccgatattgtcggtttttctacttcttcttcttgttcttcttcttcttcttcttcttcttcttcttcttcttcttcttcttcttcttccggtaatttattaattatatttctttttttatatgaattacgtttggtttgtgcagtaatttccttacatagagtgcagctttcaagaaaaatttcaatttcttcgtttgttgcataataaccactgtctttgatatgattaaacatttttgattttcttaaatgctttccttctttaatatgaaaattatcgaattctaattcattaagaacaataagctcccctaatttaaaaaattagttaaaataaattaaaatgaacatgtataaagatggattttaccattttttgaaattctaaataacttttcttcatctccaatctttttgactgaaaaacgatttttaattgaagttattgttctgtgagtgttttgaatcgcccatttctctaaatcagtttgagatagtgttttataatctgaattgttatacacaacttttgctctattaaccaaatatttaaagatttcatcatcaactgaatattttgactttacgattcttgtccaaaaaacaatttctactactatcattttttatttataaaataatttaaatacaaaaatgaatttttttttttttaaaaaaaaaaaaatttgaaaaaaaaaaaaaaaaaattttaaaaaaaaaaaaaaaaaaaaaaaaaaaaaaaaaatcaaataaaaagtaaaaaataaaaaccgaaaaacattcattgtaatttcaaatgtcgaggccggcagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcgggatccattttatttaatatactaaataataaaaaagttaaaaaatgatcattggataaattttttataattataaataaagataataattttttttttaacaaaactaaaaataaaaataataaaataattgttaaaataggttttttttttttttttttttttttaataaatggtatttattaatttatttgttgtgtgtgtttttttttttataatatttttttttttagcattgaattaagaagaaatcaaattgattctagttcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccgtccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagcttgaacatcttcaccatccattttggatcttttatattatatttatttattgattatttttttgaattaattaaaaaaaaaaaaaatttcattttataatctcagaaacctcaaaaaaaaaaaaataaaaaataaaaaatataaaaaaataaaaataaaatcccaattttaaagcgaaaaaccacccatggtttgaaaatttcaatcaatttcaaataactttacttaaaaaaaacccattttttatttaaaaa

SEQ ID NO: 126 - protein, 1172 aa, Arabidopsis thaliana
MVSGVGGSGGGRGGGRGGEEEPSSSHTPNNRRGGEQAQSSGTKSLRPRSNTESMSKAIQQYTVDARLHAVFEQSGESGKSFDYSQSLKTTTYGSSVPEQQITAYLSRIQRGGYIQPFGCMIAVDESSFRIIGYSENAREMLGIMPQSVPTLEKPEILAMGTDVRSLFTSSSSILLERAFVAREITLLNPVWIHSKNTGKPFYAILHRIDVGVVIDLEPARTEDPALSIAGAVQSQKLAVRAISQLQALPGGDIKLLCDTVVESVRDLTGYDRVMVYKFHEDEHGEVVAESKRDDLEPYIGLHYPATDIPQASRFLFKQNRVRMIVDCNATPVLVVQDDRLTQSMCLVGSTLRAPHGCHSQYMANMGSIASLAMAVIINGNEDDGSNVASGRSSMRLWGLVVCHHTSSRCIPFPLRYACEFLMQAFGLQLNMELQLALQMSEKRVLRTQTLLCDMLLRDSPAGIVTQSPSIMDLVKCDGAAFLYHGKYYPLGVAPSEVQIKDVVEWLLANHADSTGLSTDSLGDAGYPGAAALGDAVCGMAVAYITKRDFLFWFRSHTAKEIKWGGAKHHPEDKDDGQRMHPRSSFQAFLEVVKSRSQPWETAEMDAIHSLQLILRDSFKESEAAMNSKVVDGVVQPCRDMAGEQGIDELGAVAREMVRLIETATVPIFAVDAGGCINGWNAKIAELTGLSVEEAMGKSLVSDLIYKENEATVNKLLSRALRGDEEKNVEVKLKTFSPELQGKAVFVVVNACSSKDYLNNIVGVCFVGQDVTSQKIVMDKFINIQGDYKAIVHSPNPLIPPIFAADENTCCLEWNMAMEKLTGWSRSEVIGKMIVGEVFGSCCMLKGPDALTKFMIVLHNAIGGQDTDKFPFPFFDRNGKFVQALLTANKRVSLEGKVIGAFCFLQIPSPELQQALAVQRRQDTECFTKAKELAYICQVIKNPLSGMRFANSLLEATDLNEDQKQLLETSVSCEKQISRIVGDMDLESIEDGSFVLKREEFFLGSVINAIVSQAMFLLRDRGLQLIRDIPEEIKSIEVFGDQIRIQQLLAEFLLSIIRYAPSQEWVEIHLSQLSKQMADGFAAIRTEFRMACPGEGLPPELVRDMFHSSRWTSPEGLGLSVCRKILKLMNGEVQYIRESERSYFLIILELPVPRKRPLSTASGSGDMMLMMPY

SEQ ID NO: 127 - DNA, 3152 bp, Homo sapiens
tgtaaacaacttttggacacatctgggcagttgctaagggctcttgccaagcgtctagcaatacctgaacaccttctatggctgccccaaggagagctgcaacctgtttgtgctgaaggacacactaaagaagatgcagaagttctttggactgccccagacaggtgatcttgaccagaataccatcgagaccatgcggaagccacgctgcggcaacccagatgtggccaactacaacttcttccctcgcaagcccaagtgggacaagaaccagatcacatacaggatcattggctacacacctgatctggacccagagacagtggatgatgcctttgctcgtgccttccaagtctggagcgatgtgaccccactgcggttttctcgaatccatgatggagaggcagacatcatgatcaactttggccgctgggagcatggcgatggatacccctttgacggtaaggacggactcctggctcatgccttcgccccaggcactggtgttgggggagactcccattttgatgacgatgagctatggaccttgggagaaggccaagtggtccgtgtgaagtatgggaacgccgatggggagtactgcaagttccccttcttgttcaatggcaaggagtacaacagctgcactgataccggccgcagcgatggcttcctctggtgctccaccacctacaactttgagaaggatggcaagtacggcttctgtccccatgaagccctgttcaccatgggcggcaacgctgaaggacagccctgcaagtttccattccgcttccagggcacatcctatgacagctgcaccactgagggccgcacggatggctaccgctggtgcggcaccactgaggactacgaccgcgacaagaagtatggcttctgccctgagaccgccatgtccactgttggtgggaactcagaaggtgccccctgtgtcttccccttcactttcctgggcaacaaatatgagagctgcaccagcgccggccgcagtgacggaaagatgtggtgtgcgaccacagccaactacgatgatgaccgcaagtggggcttctgccctgaccaagggtacagcctgttcctcgtggcagcccacgagtttggccacgccatggggctggagcactcccaagaccctggggccctgatggcacccatttacacctacaccaagaacttccgtctgtcccaggatgacatcaagggcattcaggagctctatggggcctctcctgacattgaccttggcaccggccccacccccacgctgggccctgtcactcctgagatctgcaaacaggacattgtatttgatggcatcgctcagatccgtggtgagatcttcttcttcaaggaccggttcatttggcggactgtgacgccacgtgacaagcccatggggcccctgctggtggccacattctggcctgagctcccggaaaagattgatgcggtatacgaggccccacaggaggagaaggctgtgttctttgcagggaatgaatactggatctactcagccagcaccctggagcgagggtaccccaagccactgaccagcctgggactgccccctgatgtccagcgagtggatgccgcctttaactggagcaaaaacaagaagacatacatctttgctggagacaaattctggagatacaatgaggtgaagaagaaaatggatcctggcttccccaagctcatcgcagatgcctggaatgccatccccgataacctggatgccgtcgtggacctgcagggcggcggtcacagctacttcttcaagggtgcctattacctgaagctggagaaccaaagtctgaagagcgtgaagtttggaagcatcaaatccgactggctaggctgctgagctggccctggctcccacaggcccttcctctccactgccttcgatacaccgggcctggagaactagagaaggacccggaggggcctggcagccgtgccttcagctctacagctaatcagcattctcactcctacctggtaatttaagattccagagagtggctcctcccggtgcccaagaatagatgctgactgtactcctcccaggcgccccttccccctccaatcccaccaaccctcagagccacccctaaagagatactttgatattttcaacgcagccctgctttgggctgccctggtgctgccacacttcaggctcttctcctttcacaaccttctgtggctcacagaacccttggagccaatggagactgtctcaagagggcactggtggcccgacagcctggcacagggcagtgggacagggcatggccaggtggccactccagacccctggcttttcactgctggctgccttagaacctttcttacattagcagtttgctttgtatgcactttgtttttttctttgggtcttgttttttttttccacttagaaattgcatttcctgacagaaggactcaggttgtctgaagtcactgcacagtgcatctcagcccacatagtgatggttcccctgttcactctacttagcatgtccctaccgagtctcttctccactggatggaggaaaaccaagccgtggcttcccgctcagccctccctgcccctcccttcaaccattccccatgggaaatgtcaacaagtatgaataaagacacctactgagtggccgtgtttgccatctgttttagcagagcctagacaagggccacagacccagccagaagcggaaacttaaaaagtccgaatctctgctccctgcagggcacaggtgatggtgtctgctggaaaggtcagagcttccaaagtaaacagcaagagaacctcagggagagtaagctctagtccctctgtcctgtagaaagagccctgaagaatcagcaattttgttgctttattgtggcatctgttcgaggtttgcttcctctttaagtctgtttcttcattagcaatcatatcagttttaatgctactactaacaatgaacagtaacaataatatccccctcaattaatagagtgctttctatgtgcaaggcacttttcacgtgtcacctattttaacctttccaaccacataaataaaaaaggccattattagttgaatcttattgatgaagagaaaaaaaaaaaaaaaa

SEQ ID NO: 128 - DNA, 3159 bp, Homo sapiens
agatgttgtcttgtgagcgtgcgcgcgcctggctggaggggcactgagcctggccgcagtgttgccaatacctgaacaccttctatggctgccccaaggagagctgcaacctgtttgtgctgaaggacacactaaagaagatgcagaagttctttggactgccccagacaggtgatcttgaccagaataccatcgagaccatgcggaagccacgctgcggcaacccagatgtggccaactacaacttcttccctcgcaagcccaagtgggacaagaaccagatcacatacaggatcattggctacacacctgatctggacccagagacagtggatgatgcctttgctcgtgccttccaagtctggagcgatgtgaccccactgcggttttctcgaatccatgatggagaggcagacatcatgatcaactttggccgctgggagcatggcgatggatacccctttgacggtaaggacggactcctggctcatgccttcgccccaggcactggtgttgggggagactcccattttgatgacgatgagctatggaccttgggagaaggccaagtggtccgtgtgaagtatgggaacgccgatggggagtactgcaagttccccttcttgttcaatggcaaggagtacaacagctgcactgataccggccgcagcgatggcttcctctggtgctccaccacctacaactttgagaaggatggcaagtacggcttctgtccccatgaagccctgttcaccatgggcggcaacgctgaaggacagccctgcaagtttccattccgcttccagggcacatcctatgacagctgcaccactgagggccgcacggatggctaccgctggtgcggcaccactgaggactacgaccgcgacaagaagtatggcttctgccctgagaccgccatgtccactgttggtgggaactcagaaggtgccccctgtgtcttccccttcactttcctgggcaacaaatatgagagctgcaccagcgccggccgcagtgacggaaagatgtggtgtgcgaccacagccaactacgatgatgaccgcaagtggggcttctgccctgaccaagggtacagcctgttcctcgtggcagcccacgagtttggccacgccatggggctggagcactcccaagaccctggggccctgatggcacccatttacacctacaccaagaacttccgtctgtcccaggatgacatcaagggcattcaggagctctatggggcctctcctgacattgaccttggcaccggccccacccccacgctgggccctgtcactcctgagatctgcaaacaggacattgtatttgatggcatcgctcagatccgtggtgagatcttcttcttcaaggaccggttcatttggcggactgtgacgccacgtgacaagcccatggggcccctgctggtggccacattctggcctgagctcccggaaaagattgatgcggtatacgaggccccacaggaggagaaggctgtgttctttgcagggaatgaatactggatctactcagccagcaccctggagcgagggtaccccaagccactgaccagcctgggactgccccctgatgtccagcgagtggatgccgcctttaactggagcaaaaacaagaagacatacatctttgctggagacaaattctggagatacaatgaggtgaagaagaaaatggatcctggcttccccaagctcatcgcagatgcctggaatgccatccccgataacctggatgccgtcgtggacctgcagggcggcggtcacagctacttcttcaagggtgcctattacctgaagctggagaaccaaagtctgaagagcgtgaagtttggaagcatcaaatccgactggctaggctgctgagctggccctggctcccacaggcccttcctctccactgccttcgatacaccgggcctggagaactagagaaggacccggaggggcctggcagccgtgccttcagctctacagctaatcagcattctcactcctacctggtaatttaagattccagagagtggctcctcccggtgcccaagaatagatgctgactgtactcctcccaggcgccccttccccctccaatcccaccaaccctcagagccacccctaaagagatactttgatattttcaacgcagccctgctttgggctgccctggtgctgccacacttcaggctcttctcctttcacaaccttctgtggctcacagaacccttggagccaatggagactgtctcaagagggcactggtggcccgacagcctggcacagggcagtgggacagggcatggccaggtggccactccagacccctggcttttcactgctggctgccttagaacctttcttacattagcagtttgctttgtatgcactttgtttttttctttgggtcttgttttttttttccacttagaaattgcatttcctgacagaaggactcaggttgtctgaagtcactgcacagtgcatctcagcccacatagtgatggttcccctgttcactctacttagcatgtccctaccgagtctcttctccactggatggaggaaaaccaagccgtggcttcccgctcagccctccctgcccctcccttcaaccattccccatgggaaatgtcaacaagtatgaataaagacacctactgagtggccgtgtttgccatctgttttagcagagcctagacaagggccacagacccagccagaagcggaaacttaaaaagtccgaatctctgctccctgcagggcacaggtgatggtgtctgctggaaaggtcagagcttccaaagtaaacagcaagagaacctcagggagagtaagctctagtccctctgtcctgtagaaagagccctgaagaatcagcaattttgttgctttattgtggcatctgttcgaggtttgcttcctctttaagtctgtttcttcattagcaatcatatcagttttaatgctactactaacaatgaacagtaacaataatatccccctcaattaatagagtgctttctatgtgcaaggcacttttcacgtgtcacctattttaacctttccaaccacataaataaaaaaggccattattagttgaatcttattgatgaagagaaaaaaaaaaaaaaaa

SEQ ID NO: 129 - DNA, 3230 bp, Homo sapiens
gtgcagggtgtcctagccaagccggcgtccctcctagtagtaccgctgctctctaacctcaggacgtcaagggcctagagcgacagatgtttcccagcagggggttctgaggctgtgcgcccagatcgcgagagagcaatacctgaacaccttctatggctgccccaaggagagctgcaacctgtttgtgctgaaggacacactaaagaagatgcagaagttctttggactgccccagacaggtgatcttgaccagaataccatcgagaccatgcggaagccacgctgcggcaacccagatgtggccaactacaacttcttccctcgcaagcccaagtgggacaagaaccagatcacatacaggatcattggctacacacctgatctggacccagagacagtggatgatgcctttgctcgtgccttccaagtctggagcgatgtgaccccactgcggttttctcgaatccatgatggagaggcagacatcatgatcaactttggccgctgggagcatggcgatggatacccctttgacggtaaggacggactcctggctcatgccttcgccccaggcactggtgttgggggagactcccattttgatgacgatgagctatggaccttgggagaaggccaagtggtccgtgtgaagtatgggaacgccgatggggagtactgcaagttccccttcttgttcaatggcaaggagtacaacagctgcactgataccggccgcagcgatggcttcctctggtgctccaccacctacaactttgagaaggatggcaagtacggcttctgtccccatgaagccctgttcaccatgggcggcaacgctgaaggacagccctgcaagtttccattccgcttccagggcacatcctatgacagctgcaccactgagggccgcacggatggctaccgctggtgcggcaccactgaggactacgaccgcgacaagaagtatggcttctgccctgagaccgccatgtccactgttggtgggaactcagaaggtgccccctgtgtcttccccttcactttcctgggcaacaaatatgagagctgcaccagcgccggccgcagtgacggaaagatgtggtgtgcgaccacagccaactacgatgatgaccgcaagtggggcttctgccctgaccaagggtacagcctgttcctcgtggcagcccacgagtttggccacgccatggggctggagcactcccaagaccctggggccctgatggcacccatttacacctacaccaagaacttccgtctgtcccaggatgacatcaagggcattcaggagctctatggggcctctcctgacattgaccttggcaccggccccacccccacgctgggccctgtcactcctgagatctgcaaacaggacattgtatttgatggcatcgctcagatccgtggtgagatcttcttcttcaaggaccggttcatttggcggactgtgacgccacgtgacaagcccatggggcccctgctggtggccacattctggcctgagctcccggaaaagattgatgcggtatacgaggccccacaggaggagaaggctgtgttctttgcagggaatgaatactggatctactcagccagcaccctggagcgagggtaccccaagccactgaccagcctgggactgccccctgatgtccagcgagtggatgccgcctttaactggagcaaaaacaagaagacatacatctttgctggagacaaattctggagatacaatgaggtgaagaagaaaatggatcctggcttccccaagctcatcgcagatgcctggaatgccatccccgataacctggatgccgtcgtggacctgcagggcggcggtcacagctacttcttcaagggtgcctattacctgaagctggagaaccaaagtctgaagagcgtgaagtttggaagcatcaaatccgactggctaggctgctgagctggccctggctcccacaggcccttcctctccactgccttcgatacaccgggcctggagaactagagaaggacccggaggggcctggcagccgtgccttcagctctacagctaatcagcattctcactcctacctggtaatttaagattccagagagtggctcctcccggtgcccaagaatagatgctgactgtactcctcccaggcgccccttccccctccaatcccaccaaccctcagagccacccctaaagagatactttgatattttcaacgcagccctgctttgggctgccctggtgctgccacacttcaggctcttctcctttcacaaccttctgtggctcacagaacccttggagccaatggagactgtctcaagagggcactggtggcccgacagcctggcacagggcagtgggacagggcatggccaggtggccactccagacccctggcttttcactgctggctgccttagaacctttcttacattagcagtttgctttgtatgcactttgtttttttctttgggtcttgttttttttttccacttagaaattgcatttcctgacagaaggactcaggttgtctgaagtcactgcacagtgcatctcagcccacatagtgatggttcccctgttcactctacttagcatgtccctaccgagtctcttctccactggatggaggaaaaccaagccgtggcttcccgctcagccctccctgcccctcccttcaaccattccccatgggaaatgtcaacaagtatgaataaagacacctactgagtggccgtgtttgccatctgttttagcagagcctagacaagggccacagacccagccagaagcggaaacttaaaaagtccgaatctctgctccctgcagggcacaggtgatggtgtctgctggaaaggtcagagcttccaaagtaaacagcaagagaacctcagggagagtaagctctagtccctctgtcctgtagaaagagccctgaagaatcagcaattttgttgctttattgtggcatctgttcgaggtttgcttcctctttaagtctgtttcttcattagcaatcatatcagttttaatgctactactaacaatgaacagtaacaataatatccccctcaattaatagagtgctttctatgtgcaaggcacttttcacgtgtcacctattttaacctttccaaccacataaataaaaaaggccattattagttgaatcttattgatgaagagaaaaaaaaaaaaaaaa

SEQ ID NO: 130 - DNA, 3416 bp, Homo sapiens
aatgcatgcctgccctcctgggaatgaagcacagcaggtctcagcctcatcttacccagccccccactcaagatggaggtgcctggtttgaacacctctgacaaatggaagtctgtgttgtccagaggcaatgcagtgggggcttaagaagataactctggacttagaccgcttggcttcaaatcaaagagtgcatgaaccaaccagctggcctagtgatgatgttaggcaagtgacttctcagtttcttcatctgcaaactgggaaatttcctatctcagggttaaaagagaggtaatcttaggtgcttacctagcacatgcaatacctgaacaccttctatggctgccccaaggagagctgcaacctgtttgtgctgaaggacacactaaagaagatgcagaagttctttggactgccccagacaggtgatcttgaccagaataccatcgagaccatgcggaagccacgctgcggcaacccagatgtggccaactacaacttcttccctcgcaagcccaagtgggacaagaaccagatcacatacaggatcattggctacacacctgatctggacccagagacagtggatgatgcctttgctcgtgccttccaagtctggagcgatgtgaccccactgcggttttctcgaatccatgatggagaggcagacatcatgatcaactttggccgctgggagcatggcgatggatacccctttgacggtaaggacggactcctggctcatgccttcgccccaggcactggtgttgggggagactcccattttgatgacgatgagctatggaccttgggagaaggccaagtggtccgtgtgaagtatgggaacgccgatggggagtactgcaagttccccttcttgttcaatggcaaggagtacaacagctgcactgataccggccgcagcgatggcttcctctggtgctccaccacctacaactttgagaaggatggcaagtacggcttctgtccccatgaagccctgttcaccatgggcggcaacgctgaaggacagccctgcaagtttccattccgcttccagggcacatcctatgacagctgcaccactgagggccgcacggatggctaccgctggtgcggcaccactgaggactacgaccgcgacaagaagtatggcttctgccctgagaccgccatgtccactgttggtgggaactcagaaggtgccccctgtgtcttccccttcactttcctgggcaacaaatatgagagctgcaccagcgccggccgcagtgacggaaagatgtggtgtgcgaccacagccaactacgatgatgaccgcaagtggggcttctgccctgaccaagggtacagcctgttcctcgtggcagcccacgagtttggccacgccatggggctggagcactcccaagaccctggggccctgatggcacccatttacacctacaccaagaacttccgtctgtcccaggatgacatcaagggcattcaggagctctatggggcctctcctgacattgaccttggcaccggccccacccccacgctgggccctgtcactcctgagatctgcaaacaggacattgtatttgatggcatcgctcagatccgtggtgagatcttcttcttcaaggaccggttcatttggcggactgtgacgccacgtgacaagcccatggggcccctgctggtggccacattctggcctgagctcccggaaaagattgatgcggtatacgaggccccacaggaggagaaggctgtgttctttgcagggaatgaatactggatctactcagccagcaccctggagcgagggtaccccaagccactgaccagcctgggactgccccctgatgtccagcgagtggatgccgcctttaactggagcaaaaacaagaagacatacatctttgctggagacaaattctggagatacaatgaggtgaagaagaaaatggatcctggcttccccaagctcatcgcagatgcctggaatgccatccccgataacctggatgccgtcgtggacctgcagggcggcggtcacagctacttcttcaagggtgcctattacctgaagctggagaaccaaagtctgaagagcgtgaagtttggaagcatcaaatccgactggctaggctgctgagctggccctggctcccacaggcccttcctctccactgccttcgatacaccgggcctggagaactagagaaggacccggaggggcctggcagccgtgccttcagctctacagctaatcagcattctcactcctacctggtaatttaagattccagagagtggctcctcccggtgcccaagaatagatgctgactgtactcctcccaggcgccccttccccctccaatcccaccaaccctcagagccacccctaaagagatactttgatattttcaacgcagccctgctttgggctgccctggtgctgccacacttcaggctcttctcctttcacaaccttctgtggctcacagaacccttggagccaatggagactgtctcaagagggcactggtggcccgacagcctggcacagggcagtgggacagggcatggccaggtggccactccagacccctggcttttcactgctggctgccttagaacctttcttacattagcagtttgctttgtatgcactttgtttttttctttgggtcttgttttttttttccacttagaaattgcatttcctgacagaaggactcaggttgtctgaagtcactgcacagtgcatctcagcccacatagtgatggttcccctgttcactctacttagcatgtccctaccgagtctcttctccactggatggaggaaaaccaagccgtggcttcccgctcagccctccctgcccctcccttcaaccattccccatgggaaatgtcaacaagtatgaataaagacacctactgagtggccgtgtttgccatctgttttagcagagcctagacaagggccacagacccagccagaagcggaaacttaaaaagtccgaatctctgctccctgcagggcacaggtgatggtgtctgctggaaaggtcagagcttccaaagtaaacagcaagagaacctcagggagagtaagctctagtccctctgtcctgtagaaagagccctgaagaatcagcaattttgttgctttattgtggcatctgttcgaggtttgcttcctctttaagtctgtttcttcattagcaatcatatcagttttaatgctactactaacaatgaacagtaacaataatatccccctcaattaatagagtgctttctatgtgcaaggcacttttcacgtgtcacctattttaacctttccaaccacataaataaaaaaggccattattagttgaatcttattgatgaagagaaaaaaaaaaaaaaaa

SEQ ID NO: 131 - DNA, 3558 bp, Homo sapiens
acatctggcggctgccctcccttgtttccgctgcatccagacttcctcaggcggtggctggaggctgcgcatctggggctttaaacatacaaagggattgccaggacctgcggcggcggcggcggcggcgggggctggggcgcgggggccggaccatgagccgctgagccgggcaaaccccaggccaccgagccagcggaccctcggagcgcagccctgcgccgcggagcaggctccaaccaggcggcgaggcggccacacgcaccgagccagcgacccccgggcgacgcgcggggccagggagcgctacgatggaggcgctaatggcccggggcgcgctcacgggtcccctgagggcgctctgtctcctgggctgcctgctgagccacgccgccgccgcgccgtcgcccatcatcaagttccccggcgatgtcgcccccaaaacggacaaagagttggcagtgcaatacctgaacaccttctatggctgccccaaggagagctgcaacctgtttgtgctgaaggacacactaaagaagatgcagaagttctttggactgccccagacaggtgatcttgaccagaataccatcgagaccatgcggaagccacgctgcggcaacccagatgtggccaactacaacttcttccctcgcaagcccaagtgggacaagaaccagatcacatacaggatcattggctacacacctgatctggacccagagacagtggatgatgcctttgctcgtgccttccaagtctggagcgatgtgaccccactgcggttttctcgaatccatgatggagaggcagacatcatgatcaactttggccgctgggagcatggcgatggatacccctttgacggtaaggacggactcctggctcatgccttcgccccaggcactggtgttgggggagactcccattttgatgacgatgagctatggaccttgggagaaggccaagtggtccgtgtgaagtatgggaacgccgatggggagtactgcaagttccccttcttgttcaatggcaaggagtacaacagctgcactgataccggccgcagcgatggcttcctctggtgctccaccacctacaactttgagaaggatggcaagtacggcttctgtccccatgaagccctgttcaccatgggcggcaacgctgaaggacagccctgcaagtttccattccgcttccagggcacatcctatgacagctgcaccactgagggccgcacggatggctaccgctggtgcggcaccactgaggactacgaccgcgacaagaagtatggcttctgccctgagaccgccatgtccactgttggtgggaactcagaaggtgccccctgtgtcttccccttcactttcctgggcaacaaatatgagagctgcaccagcgccggccgcagtgacggaaagatgtggtgtgcgaccacagccaactacgatgatgaccgcaagtggggcttctgccctgaccaagggtacagcctgttcctcgtggcagcccacgagtttggccacgccatggggctggagcactcccaagaccctggggccctgatggcacccatttacacctacaccaagaacttccgtctgtcccaggatgacatcaagggcattcaggagctctatggggcctctcctgacattgaccttggcaccggccccacccccacgctgggccctgtcactcctgagatctgcaaacaggacattgtatttgatggcatcgctcagatccgtggtgagatcttcttcttcaaggaccggttcatttggcggactgtgacgccacgtgacaagcccatggggcccctgctggtggccacattctggcctgagctcccggaaaagattgatgcggtatacgaggccccacaggaggagaaggctgtgttctttgcagggaatgaatactggatctactcagccagcaccctggagcgagggtaccccaagccactgaccagcctgggactgccccctgatgtccagcgagtggatgccgcctttaactggagcaaaaacaagaagacatacatctttgctggagacaaattctggagatacaatgaggtgaagaagaaaatggatcctggcttccccaagctcatcgcagatgcctggaatgccatccccgataacctggatgccgtcgtggacctgcagggcggcggtcacagctacttcttcaagggtgcctattacctgaagctggagaaccaaagtctgaagagcgtgaagtttggaagcatcaaatccgactggctaggctgctgagctggccctggctcccacaggcccttcctctccactgccttcgatacaccgggcctggagaactagagaaggacccggaggggcctggcagccgtgccttcagctctacagctaatcagcattctcactcctacctggtaatttaagattccagagagtggctcctcccggtgcccaagaatagatgctgactgtactcctcccaggcgccccttccccctccaatcccaccaaccctcagagccacccctaaagagatactttgatattttcaacgcagccctgctttgggctgccctggtgctgccacacttcaggctcttctcctttcacaaccttctgtggctcacagaacccttggagccaatggagactgtctcaagagggcactggtggcccgacagcctggcacagggcagtgggacagggcatggccaggtggccactccagacccctggcttttcactgctggctgccttagaacctttcttacattagcagtttgctttgtatgcactttgtttttttctttgggtcttgttttttttttccacttagaaattgcatttcctgacagaaggactcaggttgtctgaagtcactgcacagtgcatctcagcccacatagtgatggttcccctgttcactctacttagcatgtccctaccgagtctcttctccactggatggaggaaaaccaagccgtggcttcccgctcagccctccctgcccctcccttcaaccattccccatgggaaatgtcaacaagtatgaataaagacacctactgagtggccgtgtttgccatctgttttagcagagcctagacaagggccacagacccagccagaagcggaaacttaaaaagtccgaatctctgctccctgcagggcacaggtgatggtgtctgctggaaaggtcagagcttccaaagtaaacagcaagagaacctcagggagagtaagctctagtccctctgtcctgtagaaagagccctgaagaatcagcaattttgttgctttattgtggcatctgttcgaggtttgcttcctctttaagtctgtttcttcattagcaatcatatcagttttaatgctactactaacaatgaacagtaacaataatatccccctcaattaatagagtgctttctatgtgcaaggcacttttcacgtgtcacctattttaacctttccaaccacataaataaaaaaggccattattagttgaatcttattgatgaagagaaaaaaaaaaaaaaaa

SEQ ID NO: 132 - DNA, 2350 bp, Bos taurus
ggcacgaggcgggctgggggccgggccatgctctgctgagccgggcaaagccgaggagaccgaatagaatagcccctcggagcgcagcgccgcgcgggggagcaggcgccagccaggcggcgacgcggccacacgcaccgagcctgccacccccgggcgacgcgcggggcccgggagcgcaatgaccgaggcgcgagtgtcccggggcgcgctggccgcccttctgcgggcgctctgcgccctgggctgcctgttgggccgtgccgccgccgcgccgtcgcccatcatcaaatttcccggcgatgtcgcccccaaaacggacaaagagttggctgtgcaatacctaaacaccttctacggctgccccaaggagagctgtaacttgtttgtgctgaaggacaccctgaagaagatgcagaagttcttcgggttaccccagacaggtgaactggaccagagcaccattgagaccatgcggaagccgcgctgtggcaaccccgacgtggccaactacaacttcttcccccgaaagcccaagtgggacaagaaccagatcacatacaggatcattggctacacacctgatctggacccccagacagtggatgatgccttcgctcgtgccttccaagtctggagcgatgtgactccgctacggttttctcggatccatgatggagaggctgacatcatgatcaactttggccgctgggagcatggagatgggtacccttttgatggcaaagacgggctcctggctcatgccttcgccccgggccctggagttgggggagattcccactttgatgacgatgagctgcggaccctgggagaaggacaagtggtccgtgtgaagtacgggaatgctgacggggaatattgcaagttccccttccggttcaacggcaaggagtacaccagctgcacagacacaggccgcagcgatggcttcctctggtgttccaccacatacaactttgacaaggacggcaagtatggcttctgcccccatgaagccctgttcaccatgggcggcaacgccgacggacagccctgcaagttcccgttccgcttccagggcacgtcttacgacagttgcaccacggagggccgcacggacggctaccgctggtgtggcaccaccgaggactacgaccgcgacaaggagtacggcttctgcccggagaccgccatgtccactgtgggcgggaactcggaaggtgccccatgtgtcctccccttcaccttcctgggcaacaagcacgagagctgcaccagcgctggccgcagtgatgggaagttgtggtgtgcgaccacctccaactacgatgatgaccgcaagtggggcttctgccccgaccaagggtacagcctgttcctggtggcagcccatgagtttggccatgcaatggggctggagcactcacaggaccctggagccctgatggcgcccatttatacctacaccaagaacttccgcctgtcccatgatgacatccagggcatccaagaactctatggggcctcccctgacattgatactggcaccggccccaccccaaccctgggccccgtcactcctgagctctgcaaacaggacatcgtcttcgacggcatctctcagatccgtggggagatcttcttcttcaaggaccgattcatctggcgaacagtgacaccacgtgacaagcccacagggcccctgctggtagccacattctggcctgagctgccggaaaagatcgatgctgtgtacgaagacccacaggaggagaaggctgtgttctttgcagggaacgaatactgggtctattcagccagcaccctggagcgagggtaccccaagccactgaccagcctggggctcccccctggtgtccagaaggtggatgctgcctttaactggagcaagaacaagaagacgtacatcttcgccggagacaaattctggagatacaatgaggtgaagaagaaaatggatcctggcttccccaagctcatcgccgatgcctggaacgccatccctgataacctggatgctgtggtggacctgcagggcgggggtcacagctacttcttcaagggcgcctattacctgaagttggagaaccaaagtctgaagagcgtgaagttcggaagcatcaaatccgattggctgggctgctgagctggctccgcctcccccagggcctgcccctccatcacctgctgcacaccagggcctgagcaccagggaaggacccgggtgggcgtggcagccctcagttctgtaattaatcagcattctcacccccacctggtaatttaagaaaccctagagtggctctgccctgtgctcaagtaaaggtga

SEQ ID NO: 133 - DNA, 1153 bp, Homo sapiens
gaaaacaccaaatcaaccataggtccaagaacaattgtctctggacggcagctatgcgactcaccgtgctgtgtgctgtgtgcctgctgcctggcagcctggccctgccgctgcctcaggaggcgggaggcatgagtgagctacagtgggaacaggctcaggactatctcaagagattttatctctatgactcagaaacaaaaaatgccaacagtttagaagccaaactcaaggagatgcaaaaattctttggcctacctataactggaatgttaaactcccgcgtcatagaaataatgcagaagcccagatgtggagtgccagatgttgcagaatactcactatttccaaatagcccaaaatggacttccaaagtggtcacctacaggatcgtatcatatactcgagacttaccgcatattacagtggatcgattagtgtcaaaggctttaaacatgtggggcaaagagatccccctgcatttcaggaaagttgtatggggaactgctgacatcatgattggctttgcgcgaggagctcatggggactcctacccatttgatgggccaggaaacacgctggctcatgcctttgcgcctgggacaggtctcggaggagatgctcacttcgatgaggatgaacgctggacggatggtagcagtctagggattaacttcctgtatgctgcaactcatgaacttggccattctttgggtatgggacattcctctgatcctaatgcagtgatgtatccaacctatggaaatggagatccccaaaattttaaactttcccaggatgatattaaaggcattcagaaactatatggaaagagaagtaattcaagaaagaaatagaaacttcaggcagaacatccattcattcattcattggattgtatatcattgttgcacaatcagaattgataagcactgttcctccactccatttagcaattatgtcacccttttttattgcagttggtttttgaatgtctttcactccttttaaggataaactcctttatggtgtgactgtgtcttattcatctatacttgcagtgggtagatgtcaataaatgttacatacacaaataaataaaatgtttattccatggtaaatttaaaaaaaaaaaaaaaaaaaaaaaaaaaaa

SEQ ID NO: 134 - DNA, 2350 bp, Bos taurus
ctcaccatgagccccctgcagcccttggtcctggcgctcctggtgctggcttgctgctctgctgtccccagacgacgccagcccaccgttgtggtctttccaggagaaccacgaaccaacctcaccaacaggcagctggcagaggaatacctgtaccgctatggctacactcctggggcagagctgagcgaggacggtcagtccctgcagcgagctctgctgcgcttccagcggcgcctgtccctgcccgagactggcgagctggacagcaccaccctgaacgccatgcgagccccgcgctgcggcgtcccagacgtgggcagattccagacctttgagggcgaactcaagtggcaccaccacaacatcacctactggatccaaaattactcggaagacctgccgcgcgccgtgatcgacgacgcctttgcccgcgctttcgcgctctggagcgctgtgacgccgctcaccttcactcgagtgtacggccccgaagctgacattgtcatccagtttggtgttagagagcacggagatgggtatcccttcgatgggaagaacgggctcctggcacacgcctttccgcctggcaaaggcattcagggagatgcccacttcgacgatgaagagttgtggtctctgggcaaaggcgttgtgatcccgacctacttcggaaacgcgaagggcgccgcctgccacttccccttcacctttgagggtcgctcctactccgcctgcaccacggacggccgttccgacgacatgctctggtgcagcaccaccgccgactacgacgccgaccgccagttcggcttctgccccagcgagagactctacacccaggacggcaatgcggacggcaagccctgcgtcttcccgttcaccttccagggccgcacctactccgcctgtacctccgatggtcgctccgacggctaccgctggtgcgccaccaccgccaactacgaccaggacaagctctacggcttctgcccgacccgagtcgatgcaacggtgaccgggggcaacgcggcgggggagctgtgcgtcttccccttcaccttcctgggcaaggaatactcggcctgcaccagagagggtcgcaatgatgggcacctctggtgcgccaccacctccaacttcgacaaagacaagaagtggggcttctgcccggatcaaggatacagcctgttccttgtggccgcacacgagtttggccacgcgctgggcttagatcacacctccgtgccagaggcgctcatgtaccccatgtacagattcacagaggagcaccccctgcatagggacgatgttcagggcatccagcatctgtatggtcctcgccctgagcctgaaccacggcctccgaccactaccaccactaccaccaccgaaccccagcccaccgctccccccacggtctgcgtcacggggcctcccaccgcccgcccctcagagggtcccactactggccccacagggcccccggcagctggccctacgggtcctcccacggctggcccttctgcggccccgacggagtccccggatccagcggaggacgtctgcaacgtggacatcttcgacgccatcgcggagattaggaaccgcttgcatttcttcaaggctgggaagtactggagactttctgagggagggggccgccgggtgcagggtcccttccttgtcaagagcaagtggcctgcgctgccccgcaagctggactccgccttcgaggatccgctcaccaagaagattttcttcttctctgggcgccaagtatgggtgtacaccggcgcgtcgttgctaggcccgaggcgtctggacaagttgggcctgggcccggaagtggcccaggtcaccggggccctcccgcgccctgagggtaaggtgctgctgttcagcgggcagagcttctggaggttcgacgtgaagacacagaaggtggatccccagagcgtcacccccgtggaccagatgttccccggggtgcccattagcacgcacgacatctttcagtaccaagagaaagcttacttctgccaggatcacttctactggcgcgtgagttcccagaatgaggtgaatcaggtggactatgtgggctacgtgaccttcgacctcctgaagtgccctgaggactagggctcccaagcctgcttcagcactgcagcgggggccccctgggggaccctgccaatagggaatgagccagtctgccggatcccaactagtggatctgttctgaaggacgaggaggaggggaggtgggctgggccctctcttcccaccttcctttcttattagaatgtatttaataaatgtggattctttaaccttaaaaaaaaaa

SEQ ID NO: 135 - DNA, 2124 bp, Artificial Sequence
  Description of Artificial Sequence Synthetic polynucleotide
atgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccgggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgacccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagccgctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactag

SEQ ID NO: 136 - DNA, 1848 bp, Arabidopsis thaliana
atgcatcattttgtccctgacttcgataccgatgatgattatgtcaacaaccataattcttctttgaatcatcttcctagaaaatccattactactatgggtgaagatgatgatcttatggagcttttatggcagaacggtcaagttgttgttcaaaaccagagacttcacaccaagaaaccttcttcttctccaccgaagcttcttccttctatggatcctcagcagcaaccttcttcagatcagaatctttttattcaagaagatgaaatgacttcttggcttcattatcctctccgtgacgatgatttctgctcagatcttctcttctccgccgcacctactgcgacggctaccgcgacggtgagtcaagtcaccgccgcgagaccgccagtatcttcgacgaatgagtcgaggccgccggtgaggaacttcatgaatttctcgaggctgagaggggattttaataacggtagaggtggtgaatctggaccgttgctttcgaaggcggttgtgagagaatctacgcaggtaagtcctagcgcaacaccgtcggcggcggcgagtgaatccggtttaacacggcggacggatggtactgacagttccgccgtagctggaggcggcgcgtataatcggaagggaaaagcagtggctatgacggcgccggcgatcgagataaccggtacatcgtcatctgtagtgtcaaagagcgaaatcgaaccggagaagacgaacgtcgatgataggaaacgaaaagagagagaagccaccactactgatgaaactgaatcccgtagcgaggaaacaaaacaagcacgtgtatcaacaacatctaccaagagatctcgtgctgctgaagttcataatctctctgaaagaaaacggagagataggatcaatgagagaatgaaagctttgcaagaacttatacctcgctgcaacaagtcagataaagcttcgatgctagatgaagctattgagtacatgaaatctcttcagcttcaaatacagatgatgtcaatgggatgtggaatgatgccaatgatgtatccgggcatgcaacagtacatgcctcatatggcgatgggtatgggtatgaaccagcctattcctcctccttccttcatgccattccccaacatgttagccgctcaaagacctttgcctacacaaactcacatggccgggtcaggaccgcaataccctgttcatgcttctgacccgtcaagagtctttgtaccgaaccagcagtatgatccaacctcgggccagcctcagtatccagctggttacacggatccatatcagcagttccgcggtctccacccgacccaaccacctcagtttcagaatcaagcaacatcgtacccaagttcgagcagggtgagtagtagtaaggaatctgaggatcacggaaaccacacaacaggttaataatgtccatggagcaacaagaagatctgttttcacaagcaaacacaatttgttatccgacccgacccaaccacctcagtttcagaatcaagcaacatcgtatccaagttcgagcagggtgagtagtagtaaggaatctgaggatcacggaaaccacacaacaggttaataatgtccatggagcaacaagaagatctgttttcacaagcaaacacaattttgagaaattgacagagagacctaacatgtatatatatcgccatctgtttcttgtttttctttggtttgttttgtcctctcttctcaggttgtatacttagagagcggtacatgtaatgatccagagatctaggaatcaatacatagaggttgcagagtcataaaaaaaaaaaaaaaaaaaaaaaaaaaaaa

SEQ ID NO: 137 - DNA, 2348 bp, Arabidopsis thaliana
gtcaagttaaagataattttggtatatatgagaaaggtatcgacaaaaaccataacgctatagatgattgtgatttgacaaaaacaccctcaaatcattgttttcagagtttttttagataaggtacagataagaaaccacctctaaaaatcaagcaatagatctcatcgcttaaaagaagagagagatcttcacttgtatgtgtcccactgattccaacacaatgtcccagaacttgccacgtgtcgttcatttcaaaagattgcagtactgttgtccctagagaatcattatctccctcgctgtaatatctttatgctcctgtcactttctgtctgtacccaaaagaagtaatgaacctctctcatcttcttcttctctgtttctttcatgttttgtgagttgtttctcaacaattttctggtctcttagagtgagaggagagagatagagagttgtgttgggcgtggaacttggactagttccacatatcaggttatatagatcttctctttcaacttctgattcgtccagaagctttcctaatctggtcagtagtactctttttatacgggtttttggttttataagatgtggctatatttggaaataactattttgcaagctttcctagattgccagaatataaaaaaagatgtttaacaagagaacggactcatggacttgctttaaattttaattattttaaaatcattctataatgattagagtaaataaactattaggactctgaattataaaattcgattttatatatgctcctccttagatctgacatggaacaccaaggttggagttttgaggagaattatagtttgtccactaatagaagatctatcaggccacaagatgaactagtggagttattatggcgagatggacaagtggttctgcagagccaaactcatagagaacaaacccaaacccagaaacaagatcatcatgaagaagccctaagatccagcacctttcttgaagatcaagaaactgtctcttggatccaataccctccagatgaagacccattcgaacccgacgacttctcctcccacttcttctcaaccatggatcccctccagagaccaacctcagagacggttaagcctaagtccagtcctgaacctcctcaagtcatggttaagcctaaggcctgtcctgaccctcctcctcaagtcatgcctcctccaaaatttaggttaacaaattcatcatcggggattagggaaacagaaatggaacagtactcggtaacgaccgttggacctagccattgcggaagcaacccatcacagaacgatctcgatgtctcaatgagtcatgatcgaagcaaaaacatagaagaaaagcttaatccgaacgcaagttcctcatcaggtggctcctctggttgcagctttggcaaagatatcaaagaaatggctagtggaagatgcatcacaaccgaccgtaagagaaaacgtataaatcacactgacgaatctgtatctctatcagatgcaatcggtaacaagtcgaaccaacgatcaggatcaaaccgaaggagtcgagcagctgaagttcataatctctccgaaaggaggaggagagataggatcaatgagagaatgaaggctttgcaagaactaatacctcactgcagtaaaactgataaagcttcgattttagacgaagccatagattatttgaaatcacttcagttacagcttcaagtgatgtggatggggagtggaatggcggcggcggcggcttcggctccgatgatgttccccggagttcaacctcagcagttcatacgtcagatacagagcccggtacagttacctcgatttccggttatggatcagtctgcaattcagaacaatcccggtttagtttgccaaaacccggtacaaaaccagatcatctccgaccggtttgctagatacatcggtgggttcccacacatgcaggccgcgactcagatgcagccgatggagatgttgagatttagttcaccggcgggacagcaaagtcaacaaccgtcgtctgtgccgacgaagaccaccgacggttctcgtttggaccactaggttggtgagccacttttttacttccttatttttggtatgtttcttttttatatctatctttctgaacatacttaaaacgttcaaggatgtattattatagagtaaacgtgcaacttcattacgttattttctgtatatgtgagtttatgtatgtcaaaatgacatgatgagattttttgtaaacaacatcttaaaaacaggacatgtgatttttgtaatcgtaaaaactttgggatgcagtttattttctaatcaaaa

SEQ ID NO: 138 - DNA, 1575 bp, Arabidopsis thaliana
atgcctctgtttgagcttttcaggctcaccaaagctaagcttgaatctgctcaagacaggaacccttctccacctgtagatgaagttgtggagctggtgtgggaaaatggtcagatatcaactcaaagtcagtcaagtagatcgaggaacattcctccaccacaagcaaactcttccagagctagagagattggaaatggctcaaagacgactatggtggacgagatccctatgtcagtgccatcactaatgacgggtttgagtcaagacgatgactttgttccatggttgaatcatcatccctcccttgatggatattgctctgatttcttgcgtgatgtgtcgtctcctgttactgtcaacgagcaagagagtgatatggcggtaaaccaaactgctttcccgttgtttcagagaagaaaggatggcaatgaatcagctcctgctgcttcttcgtcgcagtataacggtttccaatcgcattctctgtatggaagtgatagagctagagatcttcccagccaacaaaccaatccggatcggtttactcagacgcaggaaccactaattactagtaacaagcctagtttggtcaacttttcacatttcttacgccctgcaacttttgcgaagactactaataataaccttcatgacactaaagaaaagagtcctcaaagcccgccaaatgtgtttcagaccagagttcttggagctaaagactctgaagataaggttcttaacgagtctgttgcttctgctacgcctaaagataaccaaaaggcttgcctaatatcagaggactcatgtagaaaagaccaagagagtgaaaaagcagttgtatgttcttctgttggctcgggtaatagtctcgatggcccatccgaaagtccttcactttctttaaagagaaagcattcgaatattcaagacattgactgtcatagtgaagatgtggaagaagaatcaggagatggaagaaaggaagcaggtccatctcgaacgggtttgggttcaaagagaagccgctctgcagaagtgcataatctgtctgaaaggagacggcgtgataggatcaacgagaagatgcgtgccctgcaagaactcattccaaactgtaacaaggtggacaaagcttcgatgctagatgaagccatcgagtatctcaagtcactccaacttcaagtgcagatcatgtcaatggcgtctggttactatctgccaccggcggttatgttcccaccgggtatggggcattacccggcagcagctgctgcaatggcaatgggtatgggaatgccttatgcaatgggcttgcctgatttgagccgtggtggttcatcggttaaccacggaccacagttccaagtctcggggatgcaacaacaaccagtggcgatgggtattccacgtgtctctggtggtggtatctttgccggttcttcgacgattggcaatggctcgactagagatttatctggttctaaagatcaaacaacgacgaataacaacagtaacttgaaaccaataaagagaaaacaggggtcttctgatcagttttgtggatcgtcgtga

SEQ ID NO: 139 - DNA, 1544 bp, Arabidopsis thaliana
atggaacaagtgtttgctgattggaattttgaagataattttcacatgtccactaataaaagatcaatcagaccagaagatgaattagtggagctattgtggagagatggtcaagtggttttacaaagccaagctcgtagagaaccgtcagtccaagtccaaacccacaaacaagaaaccctaagaaaacccaacaatatttttcttgacaaccaagaaacagtacaaaagcctaactacgctgctctagatgatcaagaaaccgtctcctggatacaataccctccggatgacgtcatcgaccctttcgaatccgagttctcctctcatttcttctcttcgatcgatcacctcggaggtcctgagaagccacgaatgatcgaagagacagttaagcatgaggctcaagccatggctcctcctaagtttagatcctcggttataacagtcggaccgagtcattgcggcagcaaccagtcaacaaatattcatcaggccactacacttccggtttctatgagtgatagaagcaagaacgtcgaagaaagacttgacacctcgtcaggtggctcctccggttgcagctatggaaggaacaacaaagaaaccgttagtggaacaagtgtaaccattgaccgtaaaagaaaacatgttatggatgctgatcaagaatctgtgtctcaatcagatatagggttgacctcaaccgatgatcaaaccatgggcaacaaatcgagccaacggtcaggatctactcgaagaagccgtgcagctgaagttcataatctctcagaaaggaggaggagagatcggatcaatgaaagaatgaaggctcttcaagaactcatacctcactgcagcagaacagataaagcttcgatattggatgaagcaattgattacttaaaatcacttcaaatgcaactccaagtgatgtggatgggaagtggaatggcggcggcggcagcagcagcagcaagtccgatgatgtttcccggggtacaatcatctccatacattaatcagatggctatgcaaagtcagatgcaattgtctcaattcccggttatgaaccggtccgctccgcagaaccatcccggtttagtatgtctaaacccggtacagttgcagctccaagcacagaaccaaatcttatcggagcagctcgctaggtacatgggcgggattccccagatgccgccggcgggaaatcagaccgtgcaacaacaaccagcggacatgttgggatttggatctccggcgggaccgcaaagtcaactgtcggcaccggcgaccaccgacagtcttcatatgggtaaaataggctgacttggcatatagttttcctccgaaattattcttcttacagttggtgattgttatttatttttggtcgcctaagcaagcataaaagctaagtcaaatgtattatagagatctaataagttagtctcatacttataacttatttttaaacagttgaattatagtatcaatcaagtgttgggacccgtaaaaaaaaaaaaaaaaaaaaaaaaaaaaaa

SEQ ID NO: 140 - DNA, 1189 bp, Arabidopsis thaliana
gaagaaataacttttggaacattcaacaagacaacaaaatatgacttccccatcatccaccttcagaccaaattaagttcttcaatcttgtttccctgtttcacacacatatatatatatatatatatatatatatatatatgtgtgtgtttgtgtgcagacgatgatgttcttaccaaccgattattgttgcaggttaagcgatcaagagtatatggagcttgtgtttgagaatggccagattcttgcaaagggccaaagatccaacgtttctctgcataatcaacgtaccaaatcgatcatggatttgtatgaggcagagtataacgaggatttcatgaagagtatcatccatggtggtggtggtgccatcacaaatctcggggacacgcaggttgttccacaaagtcatgttgctgctgcccatgaaacaaacatgttggaaagcaataaacatgttgacgattctgagactttgaaagcttcttcatcaaagaggatgatggttgattatcataaccgaaagaagatcaagtttatacctcctgatgagcaatccgtggttgctgataggtcgttcaaattgggctttgacacttcctccgtaggtttcactgaagacagtgaaggatcgatgtatctaagcagtagtctagatgacgagtcagatgatgcgaggccacaagttcctgcaagaacaagaaaagctttggtcaaaagaaaacgaaatgcagaagcgtataattcacctgagagagacgacaacgaatcgatgttggatgaagcaatcaattatatgacaaaccttcaacttcaagttcagatgatgacgatgggtaacagatttgttacaccatcaatgatgatgcctttggggccgaactactctcagatgggtctagcaatgggtgtgggaatgcaaatgggcgaacaacagtttctgcctgcacatgttctaggagctggcttgcctgggattaatgattcagcagatatgctaaggtttcttaaccatcctggactaatgccaatgcaaaactctgcacctttcattccaacggaaaattgttccccacaatctgtccctccatcgtgcgctgctttccctaaccaaataccaaatcccaactctttgtcaaatttagatggtgcaaccttacacaagaaatcaaggaaaactaacagatga

SEQ ID NO: 141 - DNA, 561 bp, Artificial Sequence
  Description of Artificial Sequence Synthetic polynucleotide
ttggctactacacttgaacgtattgagaagaactttgtcattactgacccaagattgccagataatcccattatattcgcgtccgatagtttcttgcagttgacagaatatagccgtgaagaaattttgggaagaaactgcaggtttctacaaggtcctgaaactgatcgcgcgacagtgagaaaaattagttgggaagaaacgccaggtttctacaaggtcctgaaactgatcgcagatgccatagataaccaaacagaggtcactgttcagctgattaattatacaaagagtggtaaaaagttctggaacctctttcacttgcagcctatgcgagatcagaagggagatgtccagtactttattggggttcagttggatggaactgagcatgtccgagatgctgccgagagagagggagtcatgctgattaagaaaactgcagaaaatattgacgaggccgcaaagagactgcccgacgccaacctggcagccgcagccaagaagaaaaagctggacggaggttcagatgacgatgacaagggtggatctggtggatct

SEQ ID NO: 142 - DNA, 555 bp, Artificial Sequence
  Description of Artificial Sequence Synthetic polynucleotide
atgttagccttgaaattagcaggtcttgatatcggaagtttggctactacacttgaacgtattgagaagaactttgtcattactgacccaagattgccagataatcccattatattcgcgtccgatagtttcttgcagttgacagaatatagccgtgaagaaattttgggaagaaactgcaggtttctacaaggtcctgaaactgatcgcgcgacagtgagaaaaattagagatgccatagataaccaaacagaggtcactgttcagctgattaattatacaaagagtggtaaaaagttctggaacctctttcacttgcagcctatgcgagatcagaagggagatgtccagtactttattggggttcagttggatggaactgagcatgtccgagatgctgccgagagagagggagtcatgctgattaagaaaactgcagaaaatattgacgaggccgcaaagagactgcccgacgccaacctggcagccgcagccaagaagaaaaagctggacggaggttcagatgacgatgacaagggtggatctggtggatct

SEQ ID NO: 143 - protein, 16 aa, Unknown
  Description of Unknown biLINuS1 sequence
KRLPDANLAAPKTKRK

SEQ ID NO: 144 - protein, 16 aa, Unknown
  Description of Unknown biLINuS2 sequence
KRLPDANLAAAAKKKK

SEQ ID NO: 145 - protein, 21 aa, Unknown
  Description of Unknown biLINuS3 sequence
KKTAENIDEAAKELPAAKKKK

SEQ ID NO: 146 - protein, 15 aa, Unknown
  Description of Unknown biLINuS4 sequence
KKTAENIDPAAKKKK

SEQ ID NO: 147 - protein, 15 aa, Unknown
  Description of Unknown biLINuS5 sequence
KKTAENIDPAAKKKK

SEQ ID NO: 148 - protein, 15 aa, Unknown
  Description of Unknown biLINuS6 sequence
KRLPDANLAAAKKKK

SEQ ID NO: 149 - protein, 14 aa, Unknown
  Description of Unknown biLINuS7 sequence
KRLPDANLAAKKKK

SEQ ID NO: 150 - protein, 13 aa, Unknown
  Description of Unknown biLINuS8 sequence
KRLPDANLAKKKK

SEQ ID NO: 151 - protein, 18 aa, Unknown
  Description of Unknown biLINuS9 sequence
KRLPDANLAAAAAAKKKK

SEQ ID NO: 152 - protein, 20 aa, Unknown
  Description of Unknown biLINuS10 sequence
KRLPDANLAAAAAAAAKKKK

SEQ ID NO: 153 - protein, 18 aa, Unknown
  Description of Unknown biLINuS11 sequence
KRLPDANLAAAAKTKRKK

SEQ ID NO: 154 - protein, 16 aa, Unknown
  Description of Unknown biLINuS12 sequence
KRLPDANLAAAAKKKK

SEQ ID NO: 155 - protein, 16 aa, Unknown
  Description of Unknown biLINuS13 sequence
KRLPDANLAAAAKKKK

SEQ ID NO: 156 - protein, 16 aa, Unknown
  Description of Unknown biLINuS14 sequence
KRLPDANLAAAAKKKK

SEQ ID NO: 157 - protein, 16 aa, Unknown
  Description of Unknown biLINuS15 sequence
RKELPDANLAAAKKKK

SEQ ID NO: 158 - protein, 16 aa, Unknown
  Description of Unknown biLINuS16 sequence
KKELPDANLAAAKKKK

SEQ ID NO: 159 - protein, 20 aa, Unknown
  Description of Unknown biLINuS17 sequence
RKELPDANLAAARKTKKKIK

SEQ ID NO: 160 - protein, 15 aa, Unknown
  Description of Unknown biLINuS18 sequence
KKELPDANLAAARRR

SEQ ID NO: 161 - protein, 17 aa, Unknown
  Description of Unknown biLINuS19 sequence
KKTAENIDEAAKELRRR

SEQ ID NO: 162 - protein, 22 aa, Unknown
  Description of Unknown biLINuS20 sequence
KKTAENIDEAAKELPDANLRRR

SEQ ID NO: 163 - protein, 17 aa, Unknown
  Description of Unknown biLINuS21 sequence
KKTAENIDEAAKELRRR

SEQ ID NO: 164 - protein, 19 aa, Unknown
  Description of Unknown biLINuS22 sequence
KRLPDANLAAAAAAAKKKK

SEQ ID NO: 165 - protein, 18 aa, Unknown
  Description of Unknown biLINuS23 sequence
KKTAENIDEAAKELKKKK

SEQ ID NO: 166 - protein, 19 aa, Unknown
  Description of Unknown biLINuS24 sequence
KKTAENIDEAAKELPKKKK

SEQ ID NO: 167 - protein, 21 aa, Unknown
  Description of Unknown biLINuS25 sequence
KKTAENIDEAAKELPDAKKKK

SEQ ID NO: 168 - protein, 23 aa, Unknown
  Description of Unknown biLINuS26 sequence
KKTAENIDEAAKELPDANLKKKK

SEQ ID NO: 169 - protein, 29 aa, Unknown
  Description of Unknown biLINuS27 sequence
KKTAENIDEAAKELPDANLAAAAAAKKKK

SEQ ID NO: 170 - protein, 17 aa, Unknown
  Description of Unknown biLINuS28 sequence
RKELPDANLAAAAKKKK

SEQ ID NO: 171 - protein, 17 aa, Unknown
  Description of Unknown biLINuS29 sequence
KKELPDANLAAAAKKKK

SEQ ID NO: 172 - protein, 19 aa, Unknown
  Description of Unknown biLINuS30 sequence
RKELPDANLAAAAAAKKKK

SEQ ID NO: 173 - protein, 18 aa, Artificial Sequence
  Description of Artificial Sequence Synthetic peptide
PSTRIQQQLGQLTLENLQ

SEQ ID NO: 174 - protein, 18 aa, Artificial Sequence
  Description of Artificial Sequence Synthetic peptide
NLVDLQKKLEELELDEQQ

SEQ ID NO: 175 - protein, 26 aa, Artificial Sequence
  Description of Artificial Sequence Synthetic peptide
LALKLAGLDIGGSGGSLALKLAGLDI

SEQ ID NO: 176 - protein, 7 aa, Artificial Sequence
  Description of Artificial Sequence Synthetic peptide
ENLYFQG

SEQ ID NO: 177 - DNA, 5343 bp, Artificial Sequence
  Description of Artificial Sequence Synthetic polynucleotide
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaactagtaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatgttttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggttcttctcaccatcaccatcaccatgaaaacctgtacttccaatccaatattggaagtggataacggatccgaattcgagcgccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat